(19) United States
(12) Patent Application Publication
(10) Pub. No.: US 2016/0340736 A1
(43) Pub. Date: Nov. 24, 2016

(54) Method for Detecting Lung Cancer

(71) Applicants: SAGA UNIVERSITY, Saga (JP); ARKRAY, Inc., Kyoto (JP)

(72) Inventors: Naoko Aragane, Saga (JP); Shinya Kimura, Saga (JP); Naomi Watanabe, Saga (JP)

(73) Assignees: SAGA UNIVERSITY, Saga (JP); ARKRAY, INC., Kyoto (JP)

(21) Appl. No.: 15/108,159

(22) PCT Filed: Dec. 25, 2014
(86) PCT No.: PCT/JP2014/084755

§ 371 Date: Jun. 24, 2016

(30) Foreign Application Priority Data
Dec. 25, 2013 (JP) 2013-267568

(51) Int. Cl. C12Q 1/68; G01N 33/574

(52) U.S. Cl. CPC C12Q 1/6886; G01N 33/57423; C12Q 2600/156; C12Q 2600/158; G01N 2333/912; G01N 2333/70596

(57) ABSTRACT

Provided is a lung cancer biomarker containing a DDR2 protein or a DDR2 gene, or a mutant thereof, i.e., the following (a), (b) and (c): (a) overexpressed DDR2 protein or DDR2 gene; (b) overexpressed DDR2 protein or DDR2 gene mutant; and (c) DDR2 protein or DDR2 gene mutant.

### SEQUENCE LISTING VIA EFS WEB

A computer readable text file, entitled “SequenceListing.txt,” created on or about Jun. 22, 2016 with a file size of about 237 kb contains the sequence listing for this application and is hereby incorporated by reference in its entirety.

### TECHNICAL FIELD

The present invention relates to a method for examining lung cancer using a DDR2 protein, a DDR2 gene or a mutant thereof as an indicator.

### BACKGROUND ART

Primary lung cancers are diseases with poor prognosis that still account for the highest number of deaths, among the various incidence regions, in Japan. Although prognosis for adenocarcinoma that accounts for about 40% of the primary lung cancers is improving due to the recent progress in elucidation of genes as therapeutic targets, genes as therapeutic targets for squamous cell carcinoma that accounts for about 35% of the primary lung cancers have hardly been elucidated.

Recently, Peter S Hammerman et al. reported that about 3.8% of the squamous cell lung carcinomas had a mutation in discoidin domain receptor tyrosine kinase 2 (DDR2), which was associated with the effect of dasatinib, i.e., a therapeutic agent for chronic myeloid leukemia (Cancer Discovery 2011; 1(1):78-89, Non-Patent Document 1).

DDR2 is a receptor tyrosine kinase that binds with collagen as the ligand, and plays a role in cell adhesion, cell proliferation, extracellular remodeling and the like. Besides squamous cell lung carcinoma, mutations associated with 18 types of amino-acid changes at 17 sites have also been reported in large cell lung carcinoma, clear cell renal carcinoma and else, but its role in cancer cells has not yet been fully understood.

### RELATED ART DOCUMENTS

### Non-Patent Document

Non-Patent Document 1: Cancer Discovery 2011; 1(1):78-89

### SUMMARY OF THE INVENTION

### Problems to be Solved by the Invention

The present invention has an objective in providing a method for examining lung cancer using an amino acid mutation of a DDR2 protein or a mutation of a DDR2 gene as an indicator.

### Means for Solving the Problems

In order to solve the above-described problem, the present inventors have gone through keen studies, as a result of which found that overexpression of a DDR2 protein or a DDR2 gene, as well as a mutation of a predetermined amino acid residue of a DDR2 protein and a mutation of a predetermined base of a DDR2 gene are associated with lung cancer, in particular, squamous cell lung carcinoma, thereby accomplishing the present invention.

Thus, the present invention is as follows.
(1) A biomarker for lung cancer, comprising a DDR2 protein, a DDR2 gene or a mutant thereof of (a), (b) or (c) below:

(a) an overexpressed DDR2 protein or DDR2 gene;

(b) a mutant of an overexpressed DDR2 protein or DDR2 gene; or

(c) a mutant of a DDR2 protein or a DDR2 gene.
(2) The biomarker according to (1), wherein the DDR2 protein is a protein of (a) or (b) below:

(a) a protein containing the amino acid sequence represented by SEQ ID NO:3; or

(b) a protein that contains an amino acid sequence having one or several amino acids, other than the 681st amino acid, deleted, substituted or added in the amino acid sequence represented by SEQ ID NO:3, and that has DDR2 activity.
(3) The marker according to (1), wherein the DDR2 gene comprises DNA of (a) or (b) below:

(a) DNA containing the nucleotide sequence represented by SEQ ID NO:2; or

(b) DNA that hybridizes with a nucleotide sequence complementary to the nucleotide sequence represented by SEQ ID NO:2 under stringent conditions, and that codes for a protein having DDR2 activity.
(4) The biomarker according to (1), wherein the mutant of the DDR2 protein is a protein of (a) or (b) below:

(a) a protein containing the amino acid sequence represented by SEQ ID NO:5; or

(b) a protein that contains an amino acid sequence having one or several amino acids, other than the 681st amino acid, deleted, substituted or added in the amino acid sequence represented by SEQ ID NO:5, and that has DDR2 activity.
(5) The biomarker according to (4), wherein the mutation of the 681st amino acid residue is a mutation from threonine to isoleucine (T681I).
(6) The biomarker according to (1), wherein the mutant of the DDR2 gene is one shown in (a), (b) or (c) below:

(a) DNA coding for the amino acid sequence represented by SEQ ID NO:5;

(b) DNA coding for a protein that contains an amino acid sequence having one or several amino acids, other than the 681st amino acid, deleted, substituted or added in the amino acid sequence represented by SEQ ID NO:5, and that has DDR2 activity; or

(c) DNA having a mutation of the second nucleotide of the codon coding for the 681st amino acid residue of the nucleotide sequence of DDR2 gene.
(7) The biomarker according to (6), wherein the mutation of the second nucleotide is a mutation at the 148400th base of the nucleotide sequence of the DDR2 gene.
(8) The biomarker according to (6), wherein the mutation of the second nucleotide is a mutation from nucleotide C to nucleotide T.
(9) A method for examining lung cancer, comprising a step of determining an expression level of a DDR2 protein, a DDR2 gene or a mutant thereof in a test sample collected from a test subject, wherein, when the expression level indicates overexpression as compared to a control, the determination result is used as an indicator of lung cancer or a risk of lung cancer.
(10) A method for examining lung cancer, comprising a step of analyzing the presence or absence of a mutation in a DDR2 protein or a DDR2 gene in a test sample collected from a test subject, wherein, when a mutation is present in a predetermined amino acid sequence of the DDR2 protein or in a predetermined nucleotide sequence of the DDR2 gene, the analysis result is used as an indicator of lung cancer or a risk of lung cancer.
(11) The method according to (9) or (10), wherein the test sample is at least one selected from the group consisting of a surgically resected analyte, a bronchoscopic analyte and a bronchial lavage fluid analyte.
(12) A method for examining cancer, wherein a test subject is judged to have lung cancer or a risk of lung cancer when the expression level of a DDR2 protein, a DDR2 gene or a mutant thereof indicates overexpression by the method according to (9) or (11).
(13) A method for examining cancer, wherein the test subject is judged to have lung cancer or a risk of lung cancer when a mutation is present in an amino acid sequence of a DDR2 protein or in a nucleotide sequence of a DDR2 gene by the method according to (10) or (11).
(14) The method according to any one of (9)-(13), wherein an examination of lung cancer is detection of lung cancer or estimation of a nature of lung cancer.
(15) The method according to (9) or (10), wherein the mutant of the DDR2 protein results from a mutation of the 681st amino acid residue in the amino acid sequence of the DDR2.
(16) The method according to (15), wherein the mutation of the 681st amino acid residue is a mutation from threonine to isoleucine (T681I).
(17) The method according to (15), wherein the analysis of the presence or absence of the mutation of the 681st amino acid residue is an analysis of the presence or absence of a mutation of the second nucleotide of the codon coding for the 681st amino acid residue in the nucleotide sequence of the DDR2 gene.
(18) The method according to (17), wherein the mutation of the second nucleotide is a mutation from nucleotide C to nucleotide T.
(19) The method according to (9) or (10), wherein the mutation of the DDR2 gene is a mutation of the 148400th base in the nucleotide sequence of the DDR2 gene.
(20) The method according to (19), wherein when the gene mutation is T/C heterozygous, it is judged that lung cancer or a risk of lung cancer is present.
(21) The method according to any one of (9)-(20), wherein the lung cancer is squamous cell lung carcinoma.
(22) An oligonucleotide comprising a sequence of at least 10 bases containing the 148400th base of the nucleotide sequence of the DDR2 gene, or a sequence complementary thereto.
(23) The oligonucleotide according to (22), having a length of 10-2565 bases.
(24) A probe having the oligonucleotide according to (22) or (23) labeled.
(25) The probe according to (24), wherein the oligonucleotide has a length of 10-30 consecutive bases.
(26) A kit for examining lung cancer, comprising at least one selected from the group consisting of the oligonucleotides according to (22) and (23) and the probes according to (24) and (25).
(27) A method for treating lung cancer, comprising a step of administering an anticancer drug to a test subject who has been judged to have lung cancer by the method according to (12) or (13).
(28) A method for treating lung cancer, comprising a step of administering a substance that inhibits the expression of a DDR2 protein, a DDR2 gene or a mutant thereof to a patient with the lung cancer.
(29) The method according to (28), wherein the substance that inhibits the expression is a substance shown in (a) or (b) below:

(a) an antibody for a DDR2 protein or a mutant thereof; or

(b) an antisense nucleic acid, a decoy nucleic acid, microRNA, siRNA or shRNA for a DDR2 gene or a mutant thereof.
(30) A pharmaceutical composition for treating lung cancer, comprising a substance that inhibits the expression of a DDR2 protein, a DDR2 gene or a mutant thereof.
(31) The pharmaceutical composition according to (30), wherein the substance that inhibits the expression is a substance shown in (a) or (b) below:

(a) an antibody for a DDR2 protein or a mutant thereof; or

(b) an antisense nucleic acid, a decoy nucleic acid, microRNA, siRNA or shRNA for a DDR2 gene or a mutant thereof.

### Effect of the Invention

The present invention provides a method and a kit for examining lung cancer. By using the method of the present invention, a patient with lung cancer can easily and unfailingly be detected or diagnosed. Accordingly, the method of the present invention is useful for detecting lung cancer, understanding pathological condition, diagnosing, and developing a molecularly targeted therapy.

### BRIEF DESCRIPTION OF DRAWINGS

FIG. 1 gives diagrams showing a mutation of a gene in a lung cancer tissue and a lung cancer cell line.

FIG. 2 gives images showing results from an Invasion assay.

FIG. 3 gives diagrams showing results from an Invasion assay.

FIG. 4 gives images showing results from a cell transplantation experiment with metastatic mouse models.

### MODES FOR CARRYING OUT THE INVENTION

Hereinafter, the present invention will be described in detail. The following embodiments are illustrations for explaining the present invention, and the present invention should not be limited thereto. The present invention may be carried out in various embodiments without departing from the scope thereof.

The documents and patent documents such as patent publications cited herein are incorporated herein by reference. The present specification also includes the content of the specification of Japanese patent Application No. 2013-267568 (filed on Dec. 25, 2013) based on which the present application claims priority.

The present inventors found that a predetermined mutation in an amino acid sequence of a DDR2 (discoidin domain receptor tyrosine kinase 2) protein or in a nucleotide sequence of a DDR2 gene collected from a test subject is associated with lung cancer. Moreover, as the present inventors determined the expression level of a DDR2 protein, a DDR2 gene or a mutant thereof, they found that overexpression was associated with lung cancer. Specifically, they found that when a predetermined mutation is present in an amino acid sequence of a DDR2 protein or in a nucleotide sequence of a DDR2 gene collected from a test subject, this mutation can be used as an indicator representing that the test subject has lung cancer or a risk thereof, or when a DDR2 protein, a DDR2 gene or a mutant thereof is overexpressed as compared to a control, this overexpression can be used as an indicator representing that the test subject has lung cancer or a risk thereof.

Hence, the present invention provides a biomarker for lung cancer, comprising a DDR2 protein, a DDR2 gene or a mutant thereof of (a), (b) or (c) below:

(a) an overexpressed DDR2 protein or DDR2 gene;

(b) a mutant of an overexpressed DDR2 protein or DDR2 gene; or

(c) a mutant of a DDR2 protein or a DDR2 gene.

In addition, the present invention provides a method for examining lung cancer, comprising a step of determining an expression level of a DDR2 protein, a DDR2 gene or a mutant thereof in a test sample collected from a test subject, wherein, when the expression level indicates overexpression as compared to a control, the determination result is judged to represent lung cancer or a risk of lung cancer. Furthermore, the present invention provides a method for examining lung cancer, comprising a step of analyzing the presence or absence of a mutation in a DDR2 protein or a DDR2 gene in a test sample collected from a test subject, wherein, when the mutation is present in the amino acid sequence of the DDR2 protein or in the nucleotide sequence of the DDR2 gene, the analysis result is judged to represent lung cancer or a risk of lung cancer.

According to one aspect of the present invention, a method for examining lung cancer comprises a step of analyzing the presence or absence of the mutation of the 681st amino acid residue in an amino acid sequence of a DDR2 (discoidin domain receptor tyrosine kinase 2) collected from a test subject to examine lung cancer of the test subject based on this analysis result.

The present inventors used a total of 81 surgically resected analytes and bronchoscopic analytes as well as 26 lung cancer cell lines for sequencing all exon regions of DDR2. In one analyte out of the 81 clinical analyte and in one cell line out of the 26 cell lines, a mutation from threonine to isoleucine was found at the 681st amino acid residue (T681I). This mutation was found on a kinase domain, and was a previously unreported novel mutation. The present invention was accomplished by focusing on this novel mutation as a novel marker of lung cancer. Accordingly, the present invention provides a method for examining lung cancer by analyzing the presence or absence of the mutation of the 681st amino acid residue in the amino acid sequence of a DDR2 protein, or the mutation of the gene coding for the 681st amino acid residue in a DDR2 gene to correlate this analysis result with lung cancer of a test subject.

According to the present invention, “examination” of lung cancer refers to detection of lung cancer and/or estimation of a nature of lung cancer using the expression level of a DDR2 protein, a DDR2 gene or a mutant thereof as an indicator, or using the presence of the mutation in a DDR2 protein or a DDR2 gene as an indicator.

Specifically, it refers to detection of lung cancer and/or estimation of a nature of lung cancer by using a mutation of the 681st amino acid residue in an amino acid sequence of DDR2 (for example, SEQ ID NO:3) as an indicator, or by using a mutation in a nucleotide sequence of a gene coding for DDR2, for example, a mutation of the 148400th base in the nucleotide sequence represented by SEQ ID NO:1 or a mutation of the 2042nd base in the nucleotide sequence represented by SEQ ID NO:2, as an indicator. The term “examination” also includes an aspect of analysis and an aspect of mutational analysis.

Herein, the ordinal numerals “681st” and “148400th” represent the positions of the amino acid residue and the base targeted for the analysis, respectively, by numbers in the full-length sequences of the DDR2 protein and the DDR2 gene. Therefore, even when a DDR2 protein or a DDR2 gene used in the actual analysis is, for example, a partial fragment that is shorter than the full length, the targeted position refers to the “681st” and the “148400th”, respectively. In SEQ ID NOS:2 and 4, the 2042nd base corresponds to the above-described “148400th” base.

The phrase “to detect lung cancer” includes judgment, diagnosis or preliminary diagnosis of whether a test subject has lung cancer, and also includes judgment, diagnosis or preliminary diagnosis of whether a test subject who does not actually suffer from a lung cancer has a risk of suffering from lung cancer in the future. The term “preliminary diagnosis” also includes an aid for a physician to make a final diagnosis, in other words, an aid for diagnosing lung cancer.

Furthermore, the phrase “to estimate a nature of lung cancer” refers to estimation of the type, whether it is primary, or else of the lung cancer detected in a test subject. For example, since T681I mutation of a DDR2 protein is found in squamous cell lung carcinoma, lung cancer with the same mutation can be estimated, for example, to be squamous cell lung carcinoma. The above-described estimation also includes an aid for a physician to make a final diagnosis, in other words, an aid for diagnosing lung cancer.

1. DDR2 Protein

A protein targeted by the examination of the present invention is a wild-type protein or a mutant protein of a DDR2 protein. A wild-type DDR2 protein is targeted by an examination that uses overexpression as an indicator. A mutant protein is targeted by an examination that uses either overexpression or a mutation as an indicator.

According to the present invention, a wild-type DDR2 protein is specifically a protein of (a) or (b) below:

(a) a protein containing the amino acid sequence represented by SEQ ID NO:3; or

(b) a protein that contains an amino acid sequence having one or several amino acids, other than the (681st) amino acid at the above-described substitution site, deleted, substituted or added in the amino acid sequence represented by SEQ ID NO:3, and that has DDR2 activity.

Herein, the protein of (b) is referred to as a “modified wild-type protein”.

A mutant protein is a mutant DDR2 protein (hereinafter, also referred to as a “mutant DDR2 protein”) in which the 681st amino acid residue has been mutated from threonine to an amino acid other than threonine (for example, isoleucine) in the amino acid sequence of DDR2, which is represented by SEQ ID NO:5. In the amino acid sequence represented by SEQ ID NO:5, the mutation of the 681st amino acid residue is based on a mutation of the second nucleotide of the codon coding for the 681st amino acid residue in the nucleotide sequence of DDR2 gene (SEQ ID NO:1 or 2). In the case where the 681st amino acid residue is mutated, for example, from threonine to isoleucine, the mutation would be ACT to ATT at the 148399th-148401st bases of the nucleotide sequence represented by SEQ ID NO:1, or the mutation would be from ACT to ATT at the 2041st-2043rd bases of the nucleotide sequence represented by SEQ ID NO:2. The present invention provides both of such mutant DDR2 protein and such gene coding for said protein.

Specifically, a mutant DDR2 protein of the present invention is a protein of (a) or (b) below:

(a) a protein containing the amino acid sequence represented by SEQ ID NO:5 (a protein containing the amino acid sequence having the 681st amino acid substituted into an amino acid other than threonine in the amino acid sequence represented by SEQ ID NO:3); or

(b) a protein including an amino acid sequence having one or several amino acids, other than the 681st amino acid, deleted, substituted or added in the amino acid sequence represented by SEQ ID NO:5, and having DDR2 activity.

Herein, the protein of (b) is referred to as a “modified mutant protein”.

The above-mentioned amino acid other than threonine is not particularly limited as long as it is an amino acid other than threonine but it is preferably a hydrophobic amino acid (tryptophan, methionine, proline, phenylalanine, alanine, valine, leucine, isoleucine), and more preferably isoleucine.

2. DDR2 Gene and Mutant Thereof

A gene targeted by the examination of the present invention is a DDR2 gene (wild-type) or a mutant gene thereof. A wild-type DDR2 gene is targeted by an examination that uses overexpression as an indicator. A mutant DDR2 gene is targeted by an examination that uses either overexpression or a mutation as an indicator.

Examples of a wild-type DDR2 gene include those containing DNA of (a) or (b) below:

(a) DNA containing the nucleotide sequence represented by SEQ ID NO:1 or 2; or

(b) DNA that hybridizes with a nucleotide sequence complementary to the nucleotide sequence represented by SEQ ID NO:1 or 2 under stringent conditions, and that codes for a protein having DDR2 activity protein.

In the above-described DNA of (b), the 148399th-148401st bases of the nucleotide sequence represented by SEQ ID NO:1 (the 2041st-2043rd bases in case of SEQ ID NO:2) are identical to the DNA of (a).

Herein, this DNA of (b) is referred to as a “modified wild-type DNA”.

In addition, the present invention provides a gene coding for a mutant DDR2 protein (hereinafter, also referred to as a “mutant DDR2 gene”).

Specifically, a mutant DDR2 gene of the present invention contains DNA having a nucleotide sequence of (a), (b), (c) or (d) below:

(a) a nucleotide sequence in which the 148399th-148401st bases in the nucleotide sequence represented by SEQ ID NO:1 have been substituted into a codon of an amino acid other than threonine;

(b) DNA that hybridizes with DNA consisting of a nucleotide sequence complementary to DNA containing the nucleotide sequence of (a) above under stringent conditions (providing that the 148399th-148401st bases are identical to (a) above), and that codes for a protein having DDR2 activity;

(c) the nucleotide sequence represented by SEQ ID NO:4 (a nucleotide sequence in which the 2041st-2043 bases in the nucleotide sequence represented by SEQ ID NO:2 have been substituted into a codon of an amino acid other than threonine); or

(d) DNA that hybridizes with DNA consisting of a nucleotide sequence complementary to DNA containing the nucleotide sequence of (c) above under stringent conditions (providing that the 2041st-2043rd bases are identical to (c) above), and that codes for a protein having DDR2 activity.

Herein, DNA of (b) and (d) are referred to as “modified mutant DNA”.

Here, the phrase “under stringent conditions” refers to, but not limited to, conditions such as: (1) 5×SSC, 5× Denhardt's solution, 0.5% SDS, 50% formamide, 50° C.; (2) 0.2×SSC, 0.1% SDS, 60° C.; (3) 0.2×SSC, 0.1% SDS, 62° C.; (4) 0.2×SSC, 0.1% SDS, 65° C.; or (5) 0.1×SSC, 0.1% SDS, 65° C. Under these conditions, DNA having high sequence identity can be expected to be obtained efficiently by increasing the temperature. However, there are multiple factors that are considered to have an effect on the stringency of hybridization such as the temperature, the probe concentration, the probe length, the ion intensity, the time, the salt concentration and the like, which can suitably be selected by those skilled in the art to realize similar stringency.

The above-mentioned amino acid other than threonine is not particularly limited as long as it is an amino acid other than threonine but it is preferably a hydrophobic amino acid (tryptophan, methionine, proline, phenylalanine, alanine, valine, leucine, isoleucine), and more preferably isoleucine. While codons of isoleucine are ATA, ATC or ATT, it is preferably ATT.

The present inventors found that the genotype of the 148400th base of the DDR2 gene (SEQ ID NO:1) derived from a patient with lung cancer was T/C heterozygous.

Hence, the present invention relates to a method for examining lung cancer of an individual by using a base substitution of a DDR2 gene (T681I in the case of an amino acid sequence) as an indicator. Specifically, the present invention provides a method for examining lung cancer by analyzing a gene mutation of the 148399th-148401st bases, preferably the 148400th base, in a nucleotide sequence of a DDR2 gene collected from a test subject, or a mutation of the 2041st-2043rd bases, preferably the 2042nd base, in the nucleotide sequence represented by SEQ ID NO:2 to examine the lung cancer of the test subject based on said analysis result.

In a preferable aspect of the present invention, a mutation of the second nucleotide of the codon coding for the 681st amino acid residue of the nucleotide sequence of the DDR2 gene, that is, a mutation of the 148400th base of a nucleotide sequence (for example, SEQ ID NO:1) of a gene coding for DDR2 (also referred to as a DDR2 gene), is a mutation where the base of the nucleotide is mutated from cytosine (C) to thymine (T) (mutation from ACT to ATT). In the nucleotide sequence represented by SEQ ID NO:2, the 2042nd base is mutated from C to T.

According to the examination method based on the above-described DDR2 gene, the test subject is judged to have lung cancer or have a risk of lung cancer when the gene mutation is T/C heterozygous.

Furthermore, the test subject can be judged to have squamous cell lung carcinoma or have a risk of squamous cell lung carcinoma when the gene mutation is T/C heterozygous, where this judgment result is useful for estimating the nature of the lung cancer.

The location of the mutation of a DDR2 gene in a patient with lung cancer is not limited to a specific site and the mutation has been reported in any domains, while it is frequently found in squamous cell lung carcinoma among lung cancers (Examples). Accordingly, the above-mentioned mutation can be used as basic information that is adaptable to diagnosis or treatment of lung cancer. According to the present invention, it was confirmed that this mutation can specifically be detected in DNA derived from a sample (tissue) of a test subject.

In the present invention, genes and proteins used for the detection are DDR2 genes and mutant genes thereof, and DDR2 proteins and mutant proteins thereof (including modified proteins and modified DNA, respectively). The DDR2 gene is located in the q23,3 region of human chromosome 1, and codes for DDR2, i.e., a protein belonging to receptor tyrosine kinases. A human DDR2 gene has a nucleotide sequence with a length of 155028 bp (SEQ ID NO:1), while a DDR2 protein consists of a sequence with 855 amino acids as represented by SEQ ID NO:3 (and coded by the nucleotide sequence represented by SEQ ID NO:2). The nucleotide sequence information of human DDR2 gene and the amino acid sequence information of DDR2 protein are available, for example, under Accession No. NG-016290.

3. Biomarker

A gene or a protein that serves as a biomarker in the present invention is a DDR2 gene or a mutant gene thereof, or a DDR2 protein or a mutant protein thereof. Information of a DDR2 gene mutation refers to the mutation of the 148400th base of the nucleotide sequence of the DDR2 gene, where, for example, ACT is mutated to ATT. Since ATT is a codon of isoleucine, this mutation will result substitution from threonine to isoleucine (T681I) at position 681 of the amino acid sequence of a DDR2 protein (the 681st amino acid residue of the amino acid sequence represented by SEQ ID NO:3).

According to the present invention, a gene mutation principally includes a single-base mutation in the 148399th-148401st bases, preferably the 148400th base, of the nucleotide sequence of a DDR2 gene (SEQ ID NO:1), or the 2041st-2043rd bases, preferably the 2042nd base, of the nucleotide sequence represented by SEQ ID NO:2.

In a preferable aspect of the present invention, the above-described mutation of a DDR2 gene or a DDR2 protein can be utilized as a marker for assessing lung cancer of a test subject. In addition, overexpression of a DDR2 gene, a DDR2 protein or a mutant thereof can also be utilized as a marker for assessing lung cancer of a test subject.

According to the present invention, the primarily targeted lung cancer is squamous cell carcinoma or adenosquamous cell carcinoma.

By analyzing this mutation or overexpression, whether or not lung cancer, in particular, squamous cell carcinoma is present can be assessed.

The T681I mutation found by the present invention can be detected in a lung cancer tissue, where the correlation between the mutation and the phenotype is such that lung cancer or a risk of lung cancer is judged to be present when the gene mutation of the 148400th base in the analyzed DDR2 gene is T/C heterozygous. According to a statistical analysis carried out by the present inventors using 107 cases, the mutation was detected in 1.9% of the lung cancer cases.

Although the detection rate of the T681I mutation found by the present invention is not high, it can be expected to be a novel target molecule for a molecular target drug by estimating from the site of the mutation. A cell having this mutation can be used to establish a system for screening a novel molecular target drug.

4. Detection Method

(1) Detection of Mutation in Mutant DDR2 Protein (Including Modified Mutant Protein, which Similarly Applies Hereafter)

According to the present invention, a DDR2 protein derived from a sample of a test subject such as a surgically resected analyte, a bronchoscopic analyte or a bronchial lavage fluid analyte (including a cancer tissue, a normal lung tissue or the like) is subjected, for example, to direct sequencing of the amino acid sequence to analyze the 681st amino acid for detecting whether it is threonine or other amino acid (isoleucine). Moreover, a sample to be used is not limited to the above-mentioned samples, and sputum, blood, urine, saliva or the like can also be used.

In order to derive a DDR2 protein from the above-mentioned sample, for example, a procedure well known in the art such as solubilization of a protein with a surfactant, dialysis, gel filtration, ion-exchange chromatography, affinity chromatography or the like may be performed in a suitable combination.

(2) Detection of Mutation in Mutant DDR2 Gene (Including Modified Mutant DNA, which Similarly Applies Hereafter)

In addition, the present invention analyzes a mutation in a nucleotide sequence of DNA derived from the above-described sample collected from the test subject.

A genome sample from a test subject that is used for the above-described mutation analysis may be extracted from a surgically resected analyte, a bronchoscopic analyte, a bronchial lavage fluid analyte or the like (including a cancer tissue, a normal lung tissue or the like). Methods for extracting and purifying genomic DNA are well known. For example, a sample can be extracted from a surgically resected analyte, a bronchoscopic lung biopsy analyte or a bronchial lavage fluid analyte collected from human using a kit such as QIAamp DNA mini kit (QIAGEN). In the case of the present invention, since a mutation exists in a coding region (open reading frame) of a DDR2 protein, mRNA or total RNA may be extracted instead of genomic DNA. In order to extract mRNA or total RNA, a commercially available kit, for example, RNeasy Protect Mini Kit from QIAGEN, RNA purification kit from Roche (kit of HighPure series) or the like can be used, whose extraction procedure is well known.

Hereinafter, an exemplary method for detecting gene mutation in the above-described test sample will be described.

(2-1) Detection using PCR Method

In order to amplify a test sample by PCR, a high fidelity DNA polymerase, for example, but not limited to, DISCOVERASE DHPLC DNA (Invitrogen) is preferably used. The primers used are designed and synthesized such that a gene mutation is contained at a predetermined position of the primers so that the targeted mutation site in the test sample is amplified.

At the end of the amplification reaction, an amplification product is detected to judge of the presence or absence of the mutation. For example, a TaqMan PCR method is a method that utilizes a fluorescently labeled allele-specific oligonucleotide and PCR reaction using a Taq DNA polymerase. An allele-specific oligonucleotide (also referred to as a TaqMan probe) used in the TaqMan PCR method can be designed based on the above-described gene mutation information. Alternatively, upon amplification using allele-specific primers, a SYBR Green PCR method can be used to incorporate a fluorescently labeled base into the amplified product so as to quantify the mutated gene.

(2-2) Detection by Nucleotide Sequencing Method

According to the present invention, a mutation can also be detected by direct sequencing. As a sequencer used for nucleotide sequencing, a commercially available ABI series (Life Technologies) or the like is used.

(2-3) Detection with DNA Microarray

A DNA microarray has nucleotide probes fixed on a support, and includes a DNA chip, a microchip, a bead array and the like. First, a polynucleotide of a test sample is isolated, amplified by PCR, and labeled with a fluorescent reporter group. Subsequently, labeled DNA/mRNA and total RNA are incubated with the array. Then, this array is inserted into a scanner to detect the hybridization pattern. The hybridization data is collected as luminescence from the fluorescent reporter group bound to the probe array (i.e., incorporated into the target sequence). A probe that completely matched the target sequence generates a signal that is stronger than one having a part that does not match the target sequence. Since the sequences and the positions of the respective probes on the array are known, the sequence of the target polynucleotide reacted with the probe array can be determined according to complementarity.

(2-4) Others

Other than the above-described method, a gene mutation can be detected by a MBP-QP method. The MBP-QP method is a method in which a gene is amplified by mutation biased PCR and then a gene mutation is detected by a quenched probe method. The MBP-QP method was established by the present inventors for the purpose of detecting other gene mutations. This method allows a gene mutation be detected easily.

(3) Detection of Overexpressed DDR2 Protein or DDR2 Gene, or Mutant Thereof

An overexpressed protein (including a modified wild-type protein, which similarly applies hereafter) may be detected, for example, by ELISA, western blotting or the like.

Moreover, an overexpressed gene (including a modified wild-type DNA, which similarly applies hereafter) can be detected, for example, by quantitative PCR, Digital PCR or the like.

The above-described procedures can also be applied to mutant proteins and mutant genes (including modified mutant proteins and modified mutant DNAs, which similarly applies hereafter).

(4) Judgment of Examination Results

When an expression level of a DDR2 protein, a DDR2 gene or a mutant thereof (collectively referred to as “DDR2s”) in a test sample collected from a test subject is overexpressed as compared to a control, the value of this expression level can be used as an indicator that represents that the test subject has lung cancer or has a risk of lung cancer. When the value of this expression level is higher than that of the control, the test subject has lung cancer or a risk of lung cancer.

Herein, the term “control” refers to an expression value or level of DDR2s derived from a healthy individual, or a standardized normal value or level based on the data of DDR2s derived from a healthy individual.

Moreover, when an amino acid sequence of a DDR2 protein or a nucleotide sequence of a DDR2 gene of a test sample collected from a test subject has the above-described mutation, the test subject is judged to have lung cancer or a risk of lung cancer.

(5) Handling of Examination Results

Now, according to the method of the present invention, samples derived from multiple test subjects are used to analyze the presence or absence of a mutation. Therefore, in order to detect lung cancer of an individual test subject, the gene mutation in genomic DNA is statistically analyzed, for example, by an allele-specific PCR method, from the results of which, the position where the individual is located or belongs to is examined to find out the possibility of lung cancer of each individual test subject. Moreover, an analysis between T681I mutation and a phenotype (lung cancer or a type thereof) can be carried out for a predetermined number of test subjects (primary population), where the resulting determined values are used as basic data that can be compared with the mutations derived from single or multiple test subjects targeted for the detection so as to correlate with the phenotype, i.e., lung cancer. The analysis results of overexpression levels of wild-type genes and proteins can also be handled in a similar manner to the above-described analysis of the presence or absence of mutations.

The above-determined data derived from the test subjects can be incorporated into the values of the above-mentioned population to reprocess the data between the mutation or the expression level and the risk level of the lung cancer. By doing so, the number of the cases of the targeted test subjects (population) can be increased to improve the accuracy of the analysis.

5. Probe or PCR Primer for Detecting Lung Cancer

Hereinafter, a probe or a PCR primer for detecting lung cancer will be described. A method for acquiring a genomic DNA sample from a targeted test subject is as described above.

According to the present invention, an oligonucleotide used as a primer and/or a probe is an oligonucleotide (hereinafter, referred to as an “oligonucleotide of the present invention”) that consists of a nucleotide sequence having at least 10 bases designed to contain an oligonucleotide containing the 148400th base of the nucleotide sequence of a DDR2 gene (for example, SEQ ID NO:1), or a nucleotide sequence complementary thereto.

A nucleotide sequence (partial) near the mutation site is shown in Table 1.

TABLE 1

| Nucleotide sequence near mutation site | |
|---|---|
| 5′...tgtacgca c/t tgtcag...3′ | SEQ ID NO: 6 |
| ↑ | |
| 148400 | |

In Table 1, “c/t” means that “C” was mutated to “T”. The present invention provides an oligonucleotide having 10-50 bases that contains the mutation site (c/t) of the above-described nucleotide sequence of the DDR2 gene (SEQ ID NO:1). These oligonucleotides may be used as a probe or a PCR primer for detecting lung cancer. The lung cancer can be detected using the above-described mutation as an indicator.

The length of the oligonucleotide of the present invention is not particularly limited as long as it has at least 10 consecutive bases, preferably a length of 10-2565 bases, more preferably a length of 10-100 bases, still more preferably a length of 10-50 bases, and yet still more preferably a length of 10-30 bases. Preferably, an oligonucleotide having a length of 10-2565 bases is designed and synthesized from the region (SEQ ID NO:2 or 4) coding for DDR2 in the nucleotide sequence represented by SEQ ID NO:1.

The oligonucleotide of the present invention can be obtained by a general chemical synthesis based on the nucleotide sequence represented by SEQ ID NO:1 or 2. According to the present invention, a complementary strand (complementary sequence) thereof are also contained. In addition, the oligonucleotides of the present invention can be designed and synthesized based on the information of the above-described nucleotide sequence represented by SEQ ID NO:4. In this case, the mutation site may be designed to lie on the 5′ end or the 3′ end of the nucleotide sequence, but it is not limited thereto and may be designed to lie inside the 5′ or 3′ end.

In addition, the probe of the present invention may contain a labeling moiety bound to the oligonucleotide of the present invention. While the label may be a fluorescent label, a radioisotope label, a digoxigenin (DIG) label or the like, but it is preferably a fluorescent label.

When the prepared oligonucleotide is used as a probe, whether or not this probe has hybridized with the test DNA can be utilized to determine or detect the mutation.

The above-described fluorescent dye is not particularly limited. Examples include fluorescein, phosphor, rhodamine and a polymethine dye derivative. Examples of commercially available products of these fluorescent dyes include Pacific Blue, BODIPY FL (brand name, Molecular Probes), FluorePrime (trade name, Amersham Pharmacia), Fluoredite (trade name, Millipore), FAM (ABI), Cy3 and Cy5 (Amersham Pharmacia) and TAMRA (Molecular Probes). Alternatively, the probe of the present invention can be used as a TaqMan (registered trademark) probe.

Conditions for detecting a fluorescently labeled oligonucleotide are not particularly limited, and may suitably be determined according to a fluorescent dye used. For example, Pacific Blue can be detected at a detection wavelength of 445-480 nm, TAMRA can be detected at a detection wavelength of 585-700 nm, and BODIPY FL can be detected at a detection wavelength of 520-555 nm. By using a prove having such a fluorescent dye, hybridization and dissociation can easily be confirmed according to the variations of the respective fluorescence signals.

Preferably, a probe of the present invention is designed to contain the 148400th base of the nucleotide sequence represented by SEQ ID NO:1 (in the case of SEQ ID NO:2 or 4, the base that corresponds to this 148000th base), and to contain the bases both upstream and downstream from the 148400th base. Provided that the 148400th base with an allele of “C” (normal sequence) is referred to as “allele C” and the 148400th base with an allele of “T” (a sequence having a mutation at the 148400th base) is referred to as “allele T”, hybridization is performed under conditions such that the above-described probe hybridizes with the sequence of allele C while it does not hybridize with the sequence of allele T. Since the probe hybridizes with DNA having the sequence of allele T, whether the allele is C or T can be judged (detected) according to the presence or absence of a band.

In order to design an oligonucleotide as a primer for detecting allele T, they are designed from the upstream side of the 148400th base as well as from the downstream side of the 148400th base. Accordingly, fragments amplified by PCR will have different sizes depending on the presence or absence of the mutation and thus the mutation can be detected according to the difference in the sizes of the fragments. The primer is designed to have a length of at least 15 bases, preferably 15-30 bases, and more preferably 18-24 bases. Of course, the design of the primer is not limited to a region within 50 bases upstream and downstream from the 148400th base. It may suitably be selected from the region of the template DNA such that the amplified fragment is 1000 bp or less, preferably 500 bp or less, and more preferably 200 bp or less (for example, 50-100 bp) based on the sequence of genomic DNA.

While the oligonucleotide primer and the oligonucleotide probe designed as described above can chemically be synthesized by a known means and method, in general, they are synthesized using a commercially available chemical synthesizer. Additionally, an appropriate fluorescent label (for example, FAM, VIC, etc.) may be added to the probe in advance so as to allow automation of the operation.

6. Kit

The present invention provides a kit for detecting lung cancer. Specifically, the kit of the present invention includes at least one component selected from the group consisting of the nucleotide of the present invention and the probe of the present invention. For example, in addition to the above-mentioned component, the kit of the present invention may include an enzyme buffer, dNTP, a reagent as a control (for example, a tissue sample, a target oligonucleotide as a positive and negative control, or the like), a labeling and/or detecting reagent, a solid-phase support, an instruction and the like. In addition, the kit of the present invention may be a partial kit that only includes some of the required components, in which case the user can prepare the rest of the components.

The kit of the present invention can be provided as a microarray having the above-described oligonucleotide fixed on a support. The microarray has an oligonucleotide of the present invention fixed on a support, and includes a DNA chip, a microchip, a bead array and the like.

Preferably, the kit of the present invention includes an oligonucleotide that specifically hybridizes with a DNA fragment containing a mutation of a DDR2 gene.

In a case where a gene mutation or overexpression is judged with the kit of the present invention, for example, DNA containing the above-described DDR2 gene is isolated from a surgically resected analyte, a bronchoscopic lung biopsy analyte or a bronchial lavage fluid analyte collected from a human upon diagnosis or treatment of the disease, a fixed sample prepared therefrom, or the like, and then this isolated DNA is reacted with the oligonucleotide of the kit to judge a genotype or an overexpression level.

Based on the judged genotype, gene mutation or overexpression level, whether or not lung cancer (in particular, squamous cell carcinoma) or a risk of lung cancer is present is judged.

7. Substance that Inhibits Expression of DDR2

The phrase “to inhibit expression” means to suppress gene expression of DDR2, or to suppress a function of DDR2 (proliferation, migration, invasion or metastasis of a tumor cell).

Therefore, there is no limitation on the “substance” as long as it has an activity of inhibiting the expression of the DDR2 protein or the DDR2 gene. Examples of the substance that inhibits the expression of a DDR2 protein include an antibody for DDR2, and inhibitory nucleic acids for a gene coding for DDR2 such as antisense nucleic acids, decoy nucleic acids, microRNA, shRNA or siRNA.

(1) Antibody for DDR2 or Mutant Thereof

An antibody contained in a pharmaceutical composition of the present invention may be either a polyclonal antibody or a monoclonal antibody, whose preparation process is well known.

(1-1) Preparation of Polyclonal Antibody

In order to prepare the above-described antibody, first, a purified DDR2 or a purified DDR2 mutant is used as an immunogen (antigen).

Next, the obtained purified protein is dissolved in a buffer to prepare an antigen solution, to which a known adjuvant is added if necessary. Immunization is carried out by administering a solution containing the above-described purified DDR2 or a mutant thereof into a mammal animal (for example, a mouse, a rat, a rabbit, etc.).

An antiserum of interest is screened from the antisera collected from the immunized animals. The screening method can be carried out by a known immunoassay following a routine procedure. If purification of the antibody is necessary, a known method such as ammonium sulfate precipitation, ion-exchange chromatography or affinity chromatography may be employed.

(1-2) Preparation of Monoclonal Antibody

A method for preparing an antigen and a solution thereof, an immunization method, as well as the dosage, the dosing interval and the number of doses of the above-described antigen are well known in the art.

For example, an antibody-producing cell is preferably a spleen cell, a lymph node cell, a peripheral blood cell or the like, and more preferably a spleen cell.

Through cell fusion between the collected antibody-producing cell and a myeloma cell, a fusion cell (hybridoma) can be obtained.

The myeloma cell, the cell fusion technique, the mixture ratio of the antibody-producing cell and the myeloma cell, culture with a HAT selection medium, screening of the hybridoma of interest, a technique for cloning the hybridoma and else are well known in the art.

According to the present invention, a humanized antibody, a human antibody, or a fragment thereof can also be used.

A humanized antibody (CDR-grafted antibody) is a genetically-engineered monoclonal antibody, specifically an antibody whose complementarity determining region of the hypervariable region is partially or entirely a complementarity determining region of a mouse monoclonal antibody-derived hypervariable region, whose framework region of the variable region is a framework region of a human immunoglobulin-derived variable region, and whose constant region is a human immunoglobulin-derived constant region. A humanized antibody can be prepared by a method well known in the art.

A human antibody refers to an antibody whose entire regions, including the variable region of the H chain and the constant region of the H chain as well as the variable region of the L chain and the constant region of the L chain that make up an immunoglobulin, originate from a gene coding for a human immunoglobulin. A human antibody can be produced in the same manner as the above-described method for producing a polyclonal antibody or a monoclonal antibody by producing a transgenic animal that generates a human antibody, and immunizing this transgenic animal with an antigen by a method well known in the art.

An antibody fragment refers to a part containing an antigen-binding region of the above-described antibody or a part derived from this region, specific examples being F(ab′)₂, Fab′, Fab, Fv (variable fragment of antibody), scFv (single chain Fv) and dsFv (disulphide stabilized Fv).

(2) Inhibitory Nucleic Acid for Gene

An inhibitory nucleic acid for a DDR2 gene or a mutant thereof refers to a nucleic acid that suppresses the gene function or the gene expression thereof, examples being an antisense nucleic acid, siRNA, shRNA, a decoy nucleic acid and microRNA. With such an inhibitory nucleic acid, expression of the above-described gene can be suppressed.

(2-1) Antisense Nucleic Acids

Antisense nucleic acids are a single-stranded nucleic acid sequence (RNA or DNA) that can bind to a mRNA (sense) or DNA (antisense) sequence of a DDR2 gene. The length of the antisense nucleic acid sequence is at least about 17 nucleotides, and preferably 15-30 nucleotides. The antisense nucleic acids bind to the above-described gene sequence to form a double strand and suppress transcription or translation.

The antisense nucleic acids can be produced by employing a chemical synthesis method or a biochemical synthesis method known in the art.

The antisense nucleic acids can be introduced into a cell, for example, by a gene transfer method such as DNA transfection or electroporation, or by using a virus vector.

(2-2) Decoy Nucleic Acids

According to the present invention, decoy nucleic acids refer to “decoy” nucleic acids that bind to a transcription factor of a DDR2 gene and suppress the promoter activity. By introducing these nucleic acids into a cell, the transcription factor binds to these nucleic acids, by which the primary binding between the transcription factor and the genomic binding site is competitively inhibited, thereby suppressing the expression of that transcription factor. Specifically, decoy nucleic acids are nucleic acids that can bind to a target-binding sequence, or an analog thereof.

An example of preferable decoy nucleic acids of the present invention includes nucleic acids (either DNA or RNA) that can bind to a transcription factor that binds to a promoter of a DDR2 gene. The decoy nucleic acids can be designed as a single strand or a double strand including a complementary strand thereof, based on the promoter sequence of the above-described gene. The length is not particularly limited.

The decoy nucleic acids used in the present invention can be produced by employing a chemical synthesis method or a biochemical synthesis method known in the art. Alternatively, a nucleotide sequence as a template can be isolated or synthesized and then subjected to a PCR method or a gene amplification method using a cloning vector. Furthermore, nucleic acids obtained by these methods can be cleaved with, for example, a restriction enzyme or the like, and linked using a DNA ligase to produce nucleic acids of interest. Moreover, in order to obtain decoy nucleic acids that are more stable in the cell, bases can chemically be modified, for example, by alkylation or acylation. A mutant of decoy nucleic acids can also be synthesized by a method known in the art, for example, by site-directed mutagenesis or the like. Site-directed mutagenesis is well known in the art, where a commercially available kit such as GENETAILOR Site-Directed Mutagenesis System (Invitrogen), TaKaRa Site-Directed Mutagenesis System (Takara Bio) or the like can be used.

The transcription activity of the promoter upon use of the decoy nucleic acids can be analyzed by employing commonly performed luciferase assay, gel shift assay, western blotting, FACS assay, RT-PCR or the like. Kits for performing these assays are also commercially available (for example, promega dual luciferase assay kit).

(2-3) RNA Interference

According to the present invention, a synthesized small nucleic acid molecule that can regulate gene expression through RNA interference (RNAi) in the cell, for example, a siRNA, microRNA (miRNA) or shRNA molecule, can be used.

When a siRNA (small interfering RNA) molecule is used, various RNA corresponding to the target gene may be targeted. Examples of such RNA include mRNA, variants obtained by alternative splicing of the target gene, and post-transcriptionally modified RNA of the target gene. In a case where a family of transcription products that can be distinguished by alternative splicing using suitable exons arises, a siRNA molecule can be used to inhibit the expression of the exon parts or the conserved sequences.

A siRNA molecule can be designed based on a standard well known in the art. For example, as a target segment of target mRNA, a segment with 15-30 consecutive bases, preferably 19-25 bases that preferably starts from AA (most preferable), TA, GA or CA can be selected. The GC-content of the siRNA molecule is 30-70%, and preferably 35-55%.

A siRNA can be generated as a single-stranded hairpin RNA molecule that folds back on its own nucleic acids to generate a double-stranded part. A siRNA molecule can be obtained by a general chemical synthesis. Alternatively, a siRNA molecule can biologically be generated using an expression vector containing sense and antisense siRNA sequences.

Moreover, the present invention is capable of suppressing expression of the above-describe gene by using a microRNA. A microRNA (miRNA) is a single-stranded RNA with a length of about 20-25 bases that is present in a cell, and is one type of ncRNA (non coding RNA) that is considered to have a function of regulating expressions of other genes. A miRNA results by undergoing processing upon transcription into RNA, and exists as nucleic acids that form a hairpin structure that suppresses the expression of the target sequence.

Since miRNA is also an inhibitory nucleic acid based on RNAi, it can be designed and synthesized like shRNA or siRNA.

8. Therapeutic Method and Pharmaceutical Composition

The present invention provides a method for treating lung cancer, the method comprising a step of administering an anticancer drug to a test subject who has been judged to have lung cancer by the above-described method.

The present invention also provides a method for treating lung cancer, the method comprising a step of administering a substance that suppresses expression of a DDR2 protein, a DDR2 gene or a mutant thereof to a patient with lung cancer.

The present invention further provides a pharmaceutical composition for treating lung cancer, the composition comprising a substance that suppresses expression of a DDR2 protein, a DDR2 gene or a mutant thereof.

An anticancer drug or a pharmaceutical composition of the present invention may be administered into a body through a known usage, for example, but not limited to, a parenteral or oral usage, preferably by parenteral administration.

A formulation used in these various usages (a parenteral agent, an oral agent or the like) can be prepared according to a routine method by appropriately selecting and using an excipient, a filler, an extender, a binder, a wetting agent, a disintegrant, a lubricant, a surfactant, a dispersant, a buffer, a preservative, a solubilizing agent, an antiseptic, a flavoring agent, a soothing agent, a stabilizer, a tonicity agent or the like that are generally employed in pharmaceutical manufacturing.

In general, a dosage of an anticancer drug or a pharmaceutical composition of the present invention, can suitably be determined considering the mixing ratio of an active element (an antibody for DDR2 or a mutant thereof, or an inhibitory nucleic acid for DDR2 or a mutant thereof) in the formulation, as well as age and weight of the administration target (patient), stage/progress of the disease, administration route, number of doses, administration period and the like.

When an anticancer drug or a pharmaceutical composition of the present invention is used as a parenteral agent, the form thereof is generally not limited, and may be, for example, any of an intravenously injectable agent (including drip infusion), an intramuscularly injectable agent, an intraperitoneally injectable agent, a subcutaneously injectable agent, a suppository or the like.

The various injectable agents may be provided, for example, in a form of a unit dosage ampule or a multiple-dose container, or in a form of freeze-dried powder that can be redissolved in a dissolving liquid upon use. Besides the above-described active element, the parenteral agent may also contain various known diluents and additives according to its form within a range that does not impair the effect of the above-described active element. For example, the various injectable agents may contain water, glycerol, aliphatic polyalcohols such as propylene glycol or polyethylene glycol, or the like.

When used as an oral agent, the form thereof is generally not limited, and may be, for example, any of a tablet, a capsule, granule, powdered drug, a pill, a lozenge, liquid medication, a suspension agent, an emulsion formulation, syrup or the like, or a dried product that can be redissolved upon use. Besides the above-described active element, the oral agent may also contain various known diluents and additives according to its form within a range that does not impair the effect of the above-described active element. Examples include a binder (syrup, gum arabic, gelatin, sorbitol, tragacanth, polyvinylpyrrolidone, etc.), a filler (lactose, sugar, cornstarch, potato starch, calcium phosphate, sorbitol, glycine, etc.), a lubricant (magnesium stearate, talc, polyethylene glycol, silica, etc.), a disintegrant (various starches, etc.) and a wetting agent (sodium lauryl sulfate, etc.).

An oral agent can be given continuously or once to several times a day. Preferably, the administration route is parenteral (for example, intravenous injection).

When siRNA, shRNA or miRNA is given, the effective amount thereof is not particularly limited as long as the amount is sufficient to cause RNAi-mediated decomposition of the target mRNA. Those skilled in the art can readily determine the effective amount to be given to a test subject by considering factors such as the height, weight, age and sex of the test subject, the administration route, or whether it is local or systemic administration.

Hereinafter, the present invention will be described more specifically by way of examples. The present invention, however, should not be limited to these examples.

### EXAMPLE 1

Method

26 lung cancer cell lines including 9 squamous cell lung carcinoma cell lines and 80 cases of patient analytes were used for an analysis. The patient analytes were the 80 cases of surgically resected analytes and bronchoscopic analytes of primary lung cancer cases (other than adenocarcinoma) under treatments at Saga University Hospital from May, 2004 to February, 2012, among which 54 cases were diagnosed as squamous cell carcinoma. Informed consent was received from all patients. All of the analytes were subjected to all exon sequencing of DDR2 using DNA direct sequencing.

DNAs were extracted from the surgically resected analytes, the bronchopulmonary biopsy analytes and the bronchial lavage fluids with QIAamp (registered trademark) DNA mini kit (QIAGEN, Hilden, Germany). Detections of mutations were analyzed for all exons 1-19 by direct sequencing. PCR amplification was conducted using DISCOVERASE DHPLC DNA polymerase (Invitrogen Inc., CA). Thereafter, Amicon Ultra-0.5 (Millipore Inc. MA) was used for purification and direct sequencing was performed by cycle sequencing with Applied Biosystems 3130 Genetic Analyzer (Applied Biosystems, Foster City, Calif.).

Results

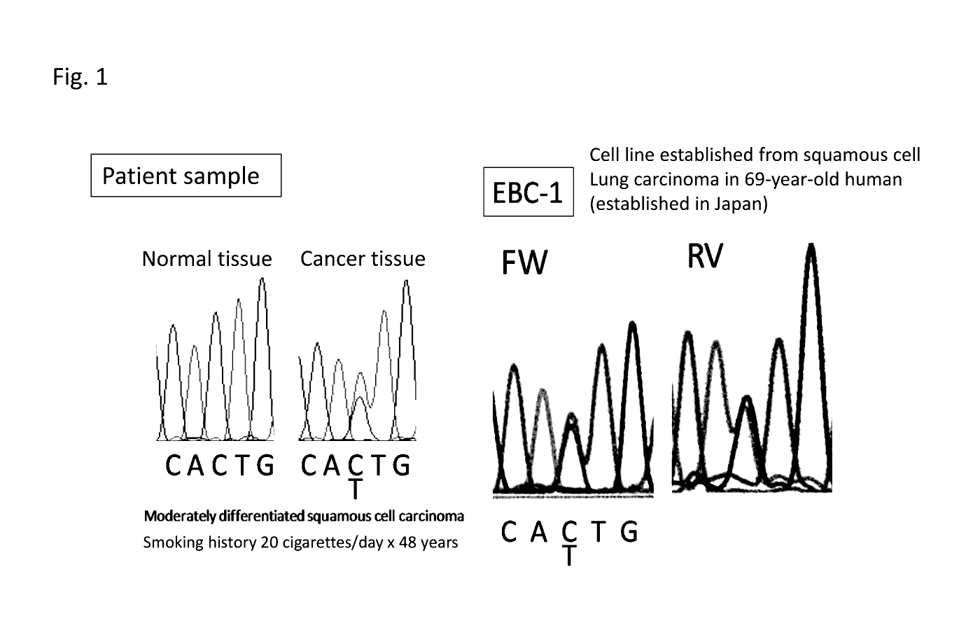

As a result of analyzing the 80 cases of patient analytes, three types of gene polymorphisms without an amino acid mutation (H136H; 11%, L420L; 17.5%, H520H; 7.5%) and one type of gene mutation associated with an amino acid mutation (T681I; 1.3%) were found (FIG. 1). Among the lung cancer cell lines, T681I mutation was also found in the squamous cell lung carcinoma cell line EBC-1, where the mutation frequency was 2.2% in whole and 3.2% among the squamous cell carcinomas (FIG. 1).

From the above-described results, a method for detecting DDR2 gene mutation (T681I) was established for diagnosing lung cancer.

### EXAMPLE 2

Invasion Assay using Cell Lines

BD BioCoat Matrigel invasion chamber 354480 was used to perform Invasion assay with the cell lines.

Cells used:

H226B-WT (D#68)

H226B-mutant (M#73)

H226B-empty (E#4)

Number of cells: 5×10⁵/well

Invasion time: 22 hours

After an invasion chamber was hydrated for 2 hours, cells were added to an insert, followed by 22 hours of incubation.

Cells on the top surface were removed and the invaded cells were fixed and stained with Diff-Quik and encapsulated.

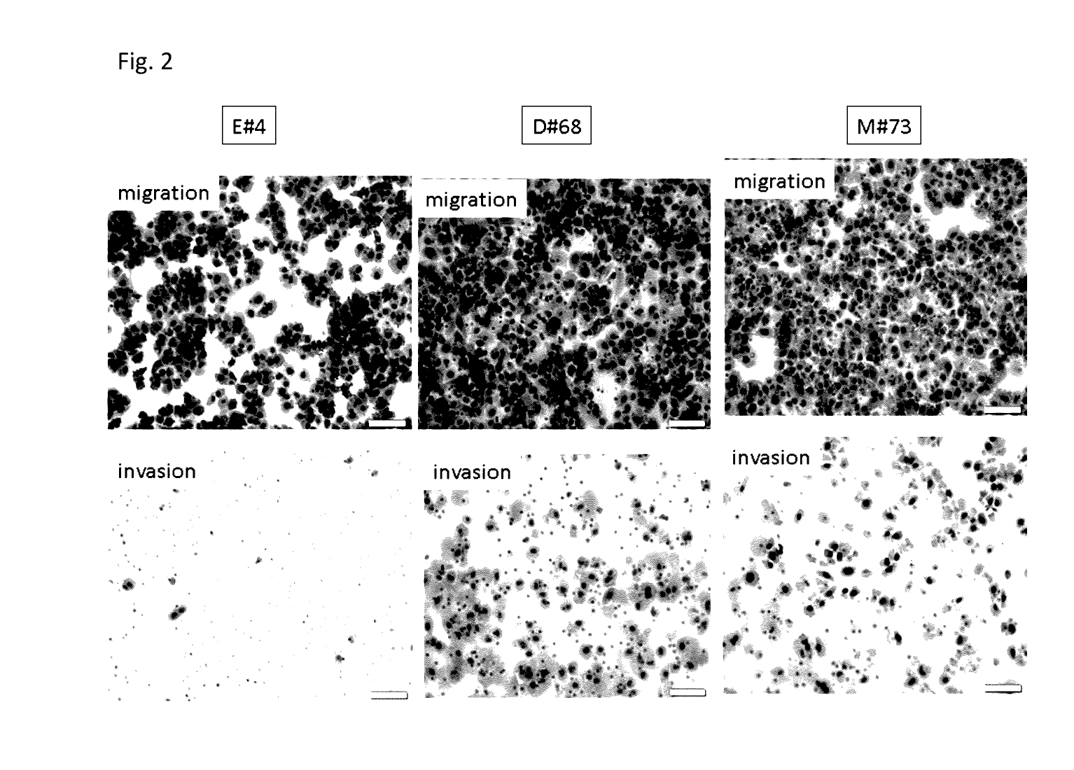

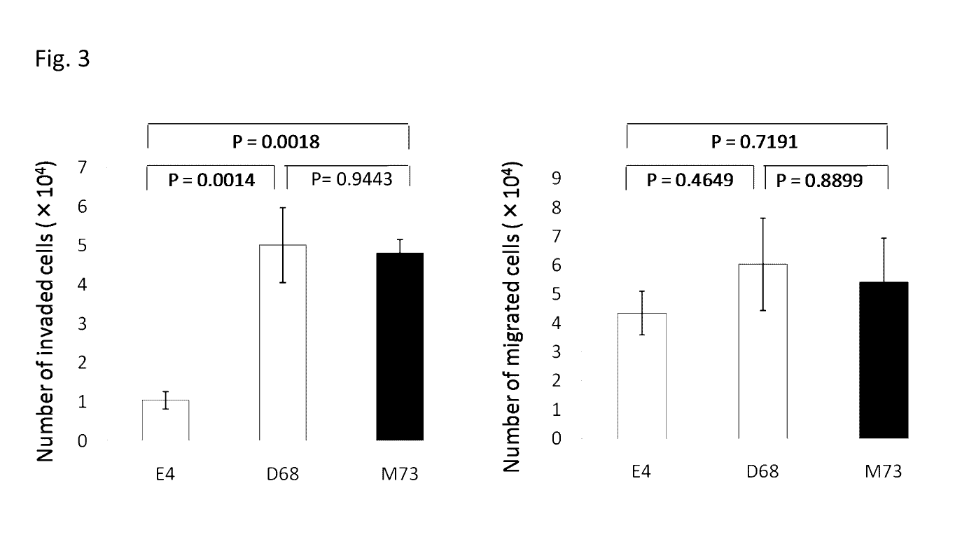

The results are shown in FIGS. 2 and 3.

As can be appreciated from FIGS. 2 and 3, when DDR2 wild-type, T681I mutation was forcibly expressed in lung cancer cell lines H226B, invasive capacity of the cancer cells was found to significantly increase.

### EXAMPLE 3

Transplantation Experiment with Metastatic Mouse Models

Method:

Mice:

8-week-old male NOJ/SCID mice

Cells:

H226B-empty E#4

H226B-wild D#68

H226B-T681I M#73

Each of the above-mentioned cells were suspended in a phosphate buffer saline, and the cells were subcutaneously transplanted into abdomens of 5 mice per group at a dose of 5×10⁵/total/100 μl. After the transplantation, the weights and the tumor masses were determined once a week. The tumor mass was calculated according to the following formula.

Tumor mass=(minor diameter)×(major diameter)/2

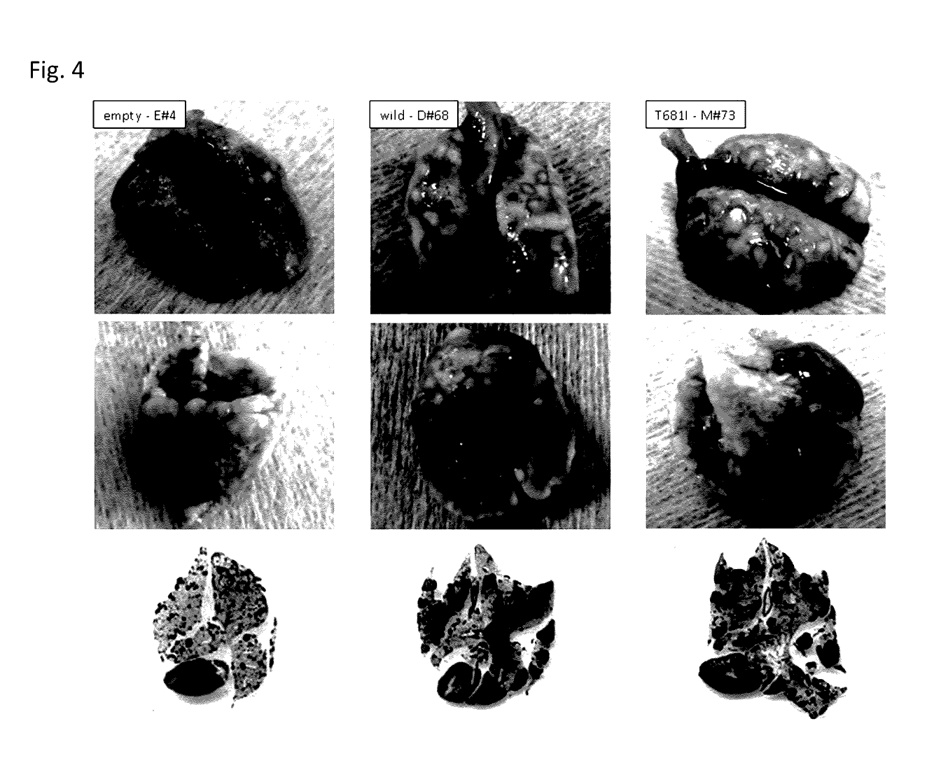

FIG. 4 gives images showing macroscopic findings of lung metastasis and results from HE staining. As can be appreciated from FIG. 4, the lung metastasis was large in total in the cell line highly expressing DDR2 (Wild type) and the cell line highly expressing mutant (T681I).

Hence, the cell line highly expressing DDR2 and the mutant of DDR2 (T681I) were shown to promote invasion/metastatic capacity of the cancer.

SEQUENCE LISTING: Free Text

SEQ ID NO:4: n represents a, g, c or t (positions of presence: 2041 . . . 2043).

SEQ ID NO:5: Xaa represents Lys, Asn, Arg, Ser, Ile, Met, Glu, Asp, Gly, Ala, Val, Gln, His, Pro, Leu, Tyr, Trp, Cys or Phe (position of presence: 681).

### INDUSTRIAL APPLICABILITY

The method and the kit of the present invention are useful for examining lung cancer.

1. (canceled)

2. The method according to claim 9, wherein the DDR2 protein is a protein of (a) or (b) below:
(a) a protein including the amino acid sequence represented by SEQ ID NO:3; or
(b) a protein that includes an amino acid sequence having one amino acid, other than the 681st amino acid, deleted, substituted or added in the amino acid sequence represented by SEQ ID NO:3, and that has DDR2 activity.

3. The method according to claim 9, wherein the DDR2 gene comprises DNA of (a) or (b) below:
(a) DNA including the nucleotide sequence represented by SEQ ID NO:2; or
(b) DNA that hybridizes with a nucleotide sequence complementary to the nucleotide sequence represented by SEQ ID NO:2 under stringent conditions, and that codes for a protein having DDR2 activity.

4. The method according to claim 9, wherein the mutant of the DDR2 protein is a protein of (a) or (b) below:
(a) a protein including the amino acid sequence represented by SEQ ID NO:5; or
(b) a protein that includes an amino acid sequence having one amino acid, other than the 681st amino acid, deleted, substituted or added in the amino acid sequence represented by SEQ ID NO:5, and that has DDR2 activity.

5. The method according to claim 4, wherein the mutation of the 681st amino acid residue is a mutation from threonine to isoleucine (T681I).

6. The method according to claim 9, wherein the mutant of the DDR2 gene is one shown in (a), (b) or (c) below:
(a) DNA coding for the amino acid sequence represented by SEQ ID NO:5;
(b) DNA coding for a protein that includes an amino acid sequence having one amino acid, other than the 681st amino acid, deleted, substituted or added in the amino acid sequence represented by SEQ ID NO:5, and that has DDR2 activity; or
(c) DNA having a mutation of the second nucleotide of the codon coding for an amino acid residue corresponding to the 681st amino acid residue in the amino acid sequence represented by SEQ ID NO: 3.

7. The method according to claim 6, wherein the mutation of the second nucleotide is a mutation at a base corresponding to the 148400th base of the nucleotide sequence represented by SEQ ID NO: 1.

8. The method according to claim 6, wherein the mutation of the second nucleotide is a mutation from nucleotide C to nucleotide T.

9. A method of detecting discoidin domain receptor tyrosine kinase 2 (DDR2), DDR2 gene, or a mutant thereof, comprising
(i) measuring an expression level of a DDR2 protein, a mutant of the DDR2 protein, a DDR2 gene, or a mutant of the DDR2 gene or (ii) detecting presence or absence of a mutation in the DDR2 protein or the DDR2 gene in a test sample collected from a test subject,
wherein overexpression of the expression level compared to a control or the presence of the mutation indicates lung cancer or a risk of lung cancer in the test subject.

10. The method according to claim 9, comprising the (ii) detecting the presence or absence of the mutation, wherein the presence of the mutation indicates lung cancer or the risk of lung cancer.

11. The method according to claim 9, wherein the test sample is at least one selected from the group consisting of a surgically resected analyte, a bronchoscopic analyte and a bronchial lavage fluid analyte.

12-13. (canceled)

14. The method according to claim 9, wherein the method detects presence or absence of lung cancer.

15. The method according to claim 9, wherein the mutant of the DDR2 protein results from a mutation at an amino acid residue corresponding to the 681st amino acid residue in the amino acid sequence represented by SEQ ID NO: 3.

16. The method according to claim 15, wherein the mutation of the 681st amino acid residue is a mutation from threonine to isoleucine (T681I).

17. The method according to claim 15, wherein the (ii) detecting detects presence or absence of a mutation of the second nucleotide of the codon coding for the 681st amino acid residue.

18. The method according to claim 17, wherein the mutation of the second nucleotide is a mutation from nucleotide C to nucleotide T.

19. The method according to claim 9, wherein the mutation of the DDR2 gene is a mutation of a base corresponding to the 148400th base of the nucleotide sequence represented by SEQ ID NO: 1.

20. The method according to claim 19, wherein when the gene mutation is T/C heterozygous, it is judged that lung cancer or the risk of lung cancer is present.

21. The method according to claim 9, wherein the lung cancer is squamous cell lung carcinoma.

22. The method according to claim 9, wherein the (i) measuring or the (ii) detecting is performed by hybridizing an oligonucleotide to the DDR2 gene or the mutation of the DDR2 gene, wherein the oligonucleotide comprises a sequence of at least 10 bases including a base corresponding to the 148400th base of the nucleotide sequence represented by SEQ ID NO: 1, or a sequence complementary thereto.

23. The method according to claim 22, wherein the oligonucleotide has a length of 10-2565 bases.

24. The method according to claim 22, wherein the oligonucleotide is labeled.

25. The method according to claim 24, wherein the oligonucleotide has a length of 10-30 consecutive bases.

26. (canceled)

27. A method of treating lung cancer, comprising a step of administering an anticancer drug to a test subject in which lung cancer is detected by the method according to claim 9.

28. A method of treating lung cancer, comprising a step of administering a substance that inhibits the expression of a DDR2 protein, a DDR2 gene or a mutant thereof to a test subject in which lung cancer is detected by the method according to claim 9.

29. The method according to claim 28, wherein the substance that inhibits the expression is a substance shown in (a) or (b) below:
(a) an antibody for a DDR2 protein or a mutant thereof; or
(b) an antisense nucleic acid, a decoy nucleic acid, microRNA, siRNA or shRNA for a DDR2 gene or a mutant thereof.

30-31. (canceled)

32. The method according to claim 9, comprising the (ii) measuring the expression level, wherein the overexpression of the expression level compared to a control indicates lung cancer or the risk of lung cancer.


SEQUENCE LISTING (6 sequences)

SEQ ID NO: 1 - DNA, 155028 bp, Homo sapiens
aaactaagaattaactatttcctaactgctctgaaatttttcctgtacactacagaaacacacagcatattattgtcacacagacacacattttgttgtcacacatacacacacgtgttctagctctggcaggtatgatcttgggcaaattaacttctctgtgcattggttttctcatatggccattgggaaaataatagtacccatcccataaaattcttgtgagtgttaaatgaaataataaatgtggaattcttataacagtgtctggtatacagtgtcagaagttttatatgtcaaaagttctgctttatatatcctgaatctcccctcctgctaattgtactcaataaattattatttctggatcttatttgatcttcacaaccctgtgaattttcagttcatattgtcccccattttacaattagaaacgtgaatgacttgttcaaggtgactctgccccctaggggtttgtaattggatcactttccatgaaagtgtccaacaggagaacagcacctagtagtgtgcagggttaggacccagaagataaagtgaacactctcttagagttaaaattatctttgaaagacctttaaatcacttacagtatacatacttctgtggatatgacttcataggataattcttaaggacactaaaattcgagaatcgtggggtaaattcaggaagacactaaaagcaagtctatatgcagctgtctgaacattcagactattttgctttttagaaaactaaggtgtttctattgtctacaagagttcctccacactgatttttaaagactattaagcctttatgccttaatatatgtcaatggatttgtctattatggatagaacaaccaggaacagggagtcttaagaattaggttctaaaattggaggccgggcgcggtggctcatgcttgtaatcctagcactgtgggaggctgaggcgggtggatcacttgaggtcagcggtttgagagcagcctggccagcatggtgaaacaccgtctctactaaaaatacaaaaatttgctgggcgtggtggtgtgcacctgtaatcccagctactcaggaggctgaggcaggagaatctcttgaacctgggaggcagaggttgctgtgagccaagaacgtgccattgcactccagcctgggcgacaagagcgaaactgtctcaaaaataaataaataaataaataaaaataaaagaaaaaagaaaaagaattaggttctaaaattggtgcttaagttaaatgtgtgcagaatataactccaaagtacacaatattctctgcttttgtgttagtaaggcataatttctttaaaatccagggattgccatgcttgtctaattattggtgtaacagacaattttggggtgtcttctaatgtatgacatcccttttctgagaattgattcatgtatgcgtatgagctcttggagccatattcatattatatgatccattgccctctacagtttagagatagaggatgcacataagaaacacatttgaccagtaaaattctccatcccagcattctagaactggactcccataatatctgtcattctctcatgatcgtttgaagcaaggagagataaatttgagaactaaggcactgttacaggcgcctgctataaccagctataatattatgcgaaaataagcagaaaaggctggtttactgaaagagaatgggctaaagcaaaatgagcagcggtaagcagaggagagagacaagaaatgaatgagagagagaagaaataagagaatgagaggagaagaaaagaggagaggaaagaagagaagagaggaggaaaggagaagagaagacaggacaggacaggagagaagaaaaacctgttgactaagttactggtggtggctaatttgtggcttaaattccttgtgaagttaaatgtaccacatgggtttggtttgtgtgaatatccatgtatccttttaaggtatgtgtgtgtttctcaaactaagttaagttaattcattttactttcatccttcattttatgtcctgaggcatcacaaaagcatccagctcatagctatttagagacagaaatattatagttatttcaatgctcaacttttgtatttacttgaaccttggtttgtagactagaaaaggtcttgaaaagtcatcttgcacatcttacttctaattaacactgcatcttgaccattccggatagatctgtcctatttttgtaaaatcccagtaatggaattctatattgtttttcagtcatttgttttcagtgtaaaacagtcctcataacaaaaatacattacttcctatggaacagaaataattctgaatgcagtgactcacatttcccaagccagaccaaccagggggtaaaatctgttcacagaagagtaataaaatggtatttctccacatctgttcctgcaatattaggaattccagtcttactcaaaactctaccaaatagctctgagagttcccaggacatttcttctgtgtgttccatgggttagtcccgtcagctttgaagttagcatttgccattttcttactttccagctctggcttcctacagcagtggctggtgtcaaggctgaacactggttgtcactcaatcagagttgtctctgaaaactgaggcttcaatctaatgaatcctcctcatgctttaaatatccctgaagtattttagtaaaatatcactttcagcttttccttgctcttttctttgtagaaccttcttcctagagttcaagattagaccccagctttggctgagaatagcctgagtgatggagatggtgttttgagtgattgatagacatgctattttcctttgagctactccttactgttgacgtagcttagtttttcttgggacttttatcccttggtcctttctgtgttcagtgagttcttccccagaagtgtcctggtctatatacaaatagactcttgactgcctagtccgcctcagacaaacccaggtgtgtctgggttttgcagtctacatcactggctcagtcctggctcaattaagactacaacttgagttctaattatacatacacagagaaaaaacaatctcacctatggagtagaaccaccttaaaatgaaaaaggaaaacacatacctatatgtacaggccctgtcctttatcaggctcagtctggttttatgaagttgttactgttttccacatttgacctatagtatcaccaaatctgtgaaatcacagattgtatttttggtacattctttatagtaatgccaaagaggtgaactactacaggcatggacatttgaggaggatttctagttgtctctggcaagcacccagtaaagtgcctgataagagcccaacacatatttattgagttgagctgcatgtctggcctgagtcaccctcaggcctgacttcagatgctgtaagttatttgaagagtcttcttaataagttgatctctgcagtgtcttttctcttcataaactaaccctagttctagttttttctctaaggtcttgcttgctgcctttaatcatttccatggctgccctctgaaccattcccatgttctccgtgtccttttctgtaacagctgtgaggcacagatgttaacggctgctttctgaccctcagactggtgctgagcttatgagacttttagggacttaacactgactcaacactagctctcagtttcatggccaccctcattgtgtgtgtgtgtgtgtgtgtgtgtgtgtgtgtgtgtgtgtgtgcgcttttccattccccttatgaaacatctcctgcccctaacttactatgggaacgttcctccatgatctctttccatcctccctttcctgtttgctcacttcttttcttgctcatcttggagactgtgcaatcccagattaactacaaacagagaagagctggtgatagctccagagctcagagaaaggaggtctctttacaagaagtctggctctcaaaggtaagctcggtcttcttgacctgccttttgccatctacctctctctttggatctcctgcccctgggtccctgcttgccctgcattctccatactcacggagaatcaggacctccagaacagcccagcgtgggcttggtcaggaatgatccaggctctgaagggttaaggctggagcctggatggatggagagggttattagttatcgtttggagagtggagtgaaggctcctcccctgttgcctacccgcccctttcacatgatctctgactatagctgtggaagcggggagaagataaaggagggaaaaaaaagttcacaaaataatttagaaagtccttataaatgcttgtgttttcatttagcagcatgaatccaggacagcaacagaccctcagaggcagcagccaactgggaattgagtggaaaaaaataggaatcctggattctggcagtttccagttgttttcttggtggggggagccgtggtgggaagggattaaatcaaatctttcgggggagagctcagctctaggctttcatggtcaaaatactaaaggtcagctccgcgtgtgtgccccaactgtcctggaagcttcaattggaaaactccaaactttaagttttgcttatagcttcagctcaagtaagaaaaatgaggtgtggggtaggcggcgggggggtggggatcacaggaaggggatcaagtcttagatggaggggaaggaagtggagatggatatgggtgggaataaggcagaagggagcggtgtctttaagtctgtcctatggttatacatattttttcttctatgaatgggttttgctgtaaattatagctttgcccatagtcccttgggtcaaaagaaatagtctccaaacagaaaacagctctgattggtgactttccttcttccctccccaacccccttcctaattgaaaccaaatgtgcaatatattggatgctgggatccctggcgctttcccaggaatcctcagaatgtgaggtttctctcaaaggcatcttgcatcagcctgtggatgtatgcctaccaccgggctccttcaccagcaaagtggaaaaagaagcgtttcacaacaaattcttctttttgggttggggaaacgcagtggattatagctctgttttcttctttccaaaactgtgcacccctggatgaaagtaagttatgtgggtcttttttctttattattttttcttttttgaaaatgaaagctccgcatatacttcttttggtgtttcaggaaaagttttaaaaaggaaacagaagggcgttttaagtcagacaaggaagggaacgtaatgaggcaccacagactcgagaaatcagatcccatgtttttgggggtctggatttatgctatgaagggaagaataaagagcagtaataaccaaagaagcctgaagcaaaataaatgaatctatagcagctgcaagatacaataaacagtgttagcagagttgaaattgttggcaggcaagacaggccgattgcagagttagtttcaggatgaagaaggcagagcaggagagctaagtttttagacatttagtagtggtgttagaaggtcctgagttttggagatagtgtagaccaagcactttttgtttttcaggggagttgctgatttacttgtgagaagggtaactttggatgaggggtggcttccccctgagatttggggtggctagggggaggctgggtgcatggaagatttttcagttgtgacagtgaaatacagcttcccagactcatctcctcgctggctctgggtgcctttccctttgttatggaatgactggggctgttttcatttgggacctctgagtcctggcctaggttcaagagtcccatccaaagggaaagagaactgttcccagtggctgaaactctcagaccagccatttgaggcaagagttcgtactgttcaagccagtagggggttcagcaccattttgccgatcattggcttcctacaacacagagttaagagtcctgaaaagggccttaggcctctcatctaaccccttcgctccacagatgtaaaaaatgataggaaggggaccgacctgttcaagtcccacagcacatctgtagtggagctgaaaatgaaattgagattctctctactgctagtccagtgttctttccactccatacctctgctttcatttcctttttaaggcttattctggctgagacaatagaggcagtatgatgagcaagtcaccacaaggacgttaattatcagtaatagagaggacgccaatggcggctcatcagctaatgatgtctatggttatatttgtgtgtgtgtgtaaaataaaaagaagctaaattctgagtattttctatgcagcaggctcaaaataatacataattcatgctcagagctcttcatacataatttgattgactctcacagctatgtaaggcattatttctatcgtacatgtaaggacattaacgcttagagaagcgaaatgagttgctcaaggaaaggtagctaatacatttgaaactcatgtatatctgactctaaaacctatgcttggaatcactattcaatgatactcccctcatctcccttgcatttggttacttttggggtcaaattccatatttcactggctgatcaggaggtgaaggtgtcaggctatgggagcagtatggattctctctcagaggccacttctgcacttggctgaacatttgtctggatatcagatgtggggttgatttctgggtctttggtatatacttacctttgttatgcaaagagcctgggacttagagtcagaagattcaggataagttctggctctaccttttactacttagcaaagttgctttgcctttctgcaccttgacttctgtatttattatatgggaacaattgcagtacctttcagtgtgattgctgcaagaggtattggacatgcatgcatgctgaaggctatcaagggcttacccatatcagctctttcgattaggatcggtaaacacagaacgtcatatgaggcagcccctgtggtgctgggtctggcagttggtgctgatgctggcaggagggcagatggatttggatgtgtgtgaaactctgaaatgggtggcctcagtcttagtgcccatctcctttgaatggaagcaggccttcctccctcccgggggaatattcctgagggctctgtcttaaattctgtctcttaaattctcctccactggcctggtttgatatgattccttgcccaaagcagtgcaggctgaaggacactgtgcttggtgaagcatcccctgtctctgaaaaggaagtgtttagtcaggaccagtcccctgattgctcctcagacaagactgacatttgaggagcttcgtatctagctgagggcagatccttgacccaggaccttcttgtcactggcaggcacatctcccttcatcctttccctggcattccctattctgtgcctgcagtcagaagggaggctgctgctcctggggtgggggtggagagccagcctgcagtggggaggcctcagcctctgtttctgggtaagatccatttgctttagaaaccttaaatgtaagggaagtgggtaaggtgtttccggaatgcctttcacttagattcctcaacagagtctccatgtccaagcacaggaccttatcacctgcttattctcccctagccactctgtgaactcgcacttttctttccttcttgtataattgctccccagtcttgaagtcatctgaattctctatcacacacagtgaatttacttcaaacctttccatgtatttgcatttttaatagacacatgaaaaaattggagcggccaccccccatacattcatgtcacaccattttcaattcgcatgagctctcagatatccttcccaagctcccttacaagatctgctttcaggcacttcacatcaccctcacctcaaacacacaatccaaacacatgtctcgcaggcatccaaaaagcaaacacacgtctaaaagtggacctccacagacatgctaacattcttgtgccagctcacataaatggtgacctgtccgcaacacagacacactggactctttatacctcttgccctgtttgaagctgtgcaacatatgaattgatcgactctgtcccaacaatgactagtcagtccagaaatgtgtccagcatccccaggcatcccagggcttctctgaatgcagcaaccatccataatgaggactgttcaccaggaaattagaaaccccaggtagaccttaaaagattttcagaccaatcaaatcagaatatctgaggttggagattgggaatcagtactttgaactgctccccaggtgattctgatgtgaaacagaattaagtacctctgctgataaatctccaactgtgttgtctttggcaattcaattttattccatttgatttagttctattcatcaaagatttagtaacactgtgctaataacatagtttagtaaagaagctgagatgtccagatacctgtcaaggatatttattggaacatcaatcacctcgtgtctaaagtgatgatctctgtaaatttttctctcttttaattatatttgtatatgtttaaatgtgctattattaagcctgcattattcatgtacgatttggctaaaaatggttctcttattttctaattgttttgcatagatatatttgatgtttcctaattttataaaccccagcatttggcaaattgtgtacttattagctggaatcttactttggctttgaaatgctgaagaaggtcagttttgagccagcccaagagttgctaaggcctggaagggcaatgcaaaggtagaggaattctaaacatgggtttggactcactacacagctcctgagtttagctgggcttctcctggaaattggatattgtctgtaatggatccactcattaagtcgtctgtggcttgtgatttgtgcctaaaagaaccccacaaaaatctgtgaggggccctttggatctatagctccgaggttagtgctggctggtgctctgtagtgtgagtgagagggagagggcatgtgataaatagaacttcagatgcgtgatgtagtgaaaagaacatgtagtgaggctctagtcccaccaccatcattttctagggtttcgacctcagaggagttttttaacttcattggtcctcaatttccccttgagtttttatccccttcatttgagtttagataagataatgtataggaaactggaaagcactgtacctgtgaagatggtaggtaatgcaagataatccttagggccttaatagcttaggtagccaaaataattgagatgggagatggattaaaaatgagggcaaataatctagcatttttgcattttttccttttaaaatttgaatgctcttatgaatccaaaaaaacatgaaacaatacaaatctcagtgaactctcatgtctaagatagttttccactgattttatttttctcttgaatagtggtgatttaaaaaaaaatcgcaggtatattttttatctattaaaaatatcatagcatcctggtaaactctgtcataattttgttttgctgtatttatttatttactcctcagtaagtagttattggatgcctagtctctgctggacactgttctatgttttatgatgaacaaaccccatgaagttacaagcatttataagcattcattgctttaggttctagggagtataatatgaatttgactgtgtgaaacttgatttagaaaattggtatccacattgggttaataaaatttagtttttaaagtttactgaactgtaatttaaaaataatacagaaatagttattaaatggtgattttcagtggagcaggtgttatgccataaataggaccccaaattttatattttttataatttttaaaaattatagagcaactttagctttacagaaaaactgagaacaaaatgcagagttctcatgaattccttctaccctttgcacaatttctctattattacaaccttgtattagtattatgttttatttgctatcagttactcaatgtgtatttattaaacataacaaaataataaataaatttattatttatgccctatttgttattatgaaaagaatacatcatattgctgcctctcaagctgtttataatgttatgtagctacagtttgaggtttctcatttattagaaaaattttttttctgtgcacttagtgcatgccttattcagtgctagggcttaacacattatctggacatagtagacatttaataaaatatctgctgagtatacagacaaatgaaatgaaggatccagaagtaaaagacgcaattcttgtccttaggaacttaggaaacaacagacacacatagcagtgactggagaaaatttcagaaatacataaaatatgattaaatctctgataaaggagacattggtataggcaaatgaggcataaagccattaacaagagccccatcctctttttcctcctttttgggactgtacttttttttaattttttttatttgtttgagacggagtctcactctgtcacccaggctggagtgcaatggtgcgatcttggctcactgcaccctctgcctcctgggttcaagtgattctcctgcctcagcctcctgagtagctgggattacagacacccgccaccaagcccagcaaatttttgtatttttagtagagttggggtttcaccaggttggccaggctggtctcaaactcctgacctcaggtgatccacctgcctcggcctcccaaagtgttgggattacaggcatgagccactgcgcccggcctgggactgtacttcttatcatcaccattgtctttgttgccatggtcttcattattatcatcataattatggctatcgttctatgtctaaatccaagcattaaagaacaaactcccatatccccatggtaaggttggagaagtgcgtgcaagtgtgtctatgtgtgtttttggcaattttaaagtacagtcacaccaatatatgccacttccatttcttgctggaagtaaaatgagtgactacttgggctttgtaaaagaaaatcgcactttggaagtccttgtgggagccataccaaaatgccttgcagtactgtaacttctcagaggcatgtaaaagttagaggattggtgaagttttcaaaagctactggcacttgtccttcaggatgaaccctcttgagatgcaggggagcaaattgacctcaataagagagtagtgggagctaaattcactcaagaatagcaatctgttcctcagacagatgcagttgtaattttagaatgtgtacataacagacagtttaaaatggacataataacgctaaaatagtatcaatggaaattaaaaataagtcctaatcccaaccaaaattctagcaagttgttttgtggatatcaacgaactgattctttttttttttcttttttctttttctatctttgagacggagtcttgctctgttgcccagactggagtgcagtggcgcaatctcggctcagtgcaacctccacctcccaggtccaagtgattctcctgtctcagcctcctgagtagctgggattacagacatgcaccaccatgactggcctcaaccagctgattctaaagtttatatagacagttaaaagaccagattagctactgcaatattgaatgagaacaaagtcaaaggactgacagtacctgacttcaaggcttactgtaaagctctggtaatcaatacattgtggtactggtgaaaaaataaacaaatagatcaagaaaacagaatagagagcccagatagacccatataatgtagccaactgttctttgacaaaggagcaaaggcaatctgatggagaaaaggtagtattttcaacaaatggtgctggaacaattggacatccacatgcaaaaataatgaatcttaggtataaaccttacatccttcacaaaagcaactcaaagtggatcatagaccaaaatgtaaatgcaaaacaatgaaactcatagaaaataacataggaaaaaatctcagtaatcttggatattgtgatgactttttagttacagcaccaaaggcactatctgtgaaagaaatccctgattagctggacttcattaaaattgtaaacttccactctgtgaaagacaatgtcaagagaatgagaatacaaaccacaggttgagaaaaatacagatggtgctgacttaaaacggttcaacttacgatttttcaactttacgacagtgcaaaagtgatactcattcagtagaaatagtacttcgaattctgaatttcattggacaaaactatggaaacagtaaaaactattcgtagttgccaggaattaggggtgagggagggataaattgaacacagaggatttttagggtagtgaaactgttctgtggttactacaatggtggatgtatgtcattatacctttgtcaaaacccatagaatgtacaacactgagagtgaaccctaagacaaacaatgtactttgggtgaaatgatttttttttttttttgagatgaagtcttgctctgtcgcccaggctggagtgcagtggtgtgatctcggctcactgcaacctctgcctcctgggttcaagcgattttcctgcctcagcctcctgagtagctgggattacaggcgtgtgctatcacgcccaactaatttttgtatttttagtacagacagggtttcaccatgttggccaggctggtttcgaactcctgacctcatgatctgcccccctcagcctcccaacgtgctgggattgcaagtgtgagccaccgcgcccagctgaaaatgatgtgttgatatcacagaccattaggtttattgataggcttattgattgtaataaatgtgccaatgtcgtgagggatgtcaatagtgagggaggttttgtagcagaggacaagggtgtatatgggaacaatctgtactttctgcttaattttgctgtgaacctacaactgctttaaaaagaattaaagtttaatttaaaaaatctgcccaatctccaaattctaatactttgattccatcttggctacttctttcttatttgaaggcagtcattgtaatatagttataatcatgagcatctgtattttaaaatgcttaattttgtttttcagataagtaccctaaacttaaatgatattattttatttattaaagctacctgaggcagaaatgtttcaaaatcttgaaaaagactaattcaataggttaatagcagataagtaggtatttttgtgtcatactcttcaaattataataactatcacttaaagaatagtcatttttgttcgtttgtgatccttccacctcagcccctcgactagctgggactatatgtgcgtactaccacacctggctaactgttgttttttgtagagatgaggtttcactatgttcttcaggcaggtctcaaactcctgggctcaagcaatcctcccaccttggcttctcaaaatgctagtattacaaacatgagccactggacccaccctcattgagctgttttaactacaacttggtatggtagaaagagtgatgggccaagggttaatagggtgagattctaattttaactttgccacctacaagcagtgggactttgtgcaacaatgttttaaatctttctgcatttaaatttttctcatctgtaaagggcaatatacaatttctgtattctccagttcagttatgctaatcgctgtcctgtcagcacccacatccccatattcactttcacagataagacttgagtcgctatgatcttcaccctagttgtcaccctgtccccatactctctgaatatgctcctctctcctctcattttagccaaataccactcttctttgaaggccagcttaagtttcagatcctccatccatcccagatcttcttggaatccctagttcttttggtcagtatcacttagtttagcaaagaccatgtcttaggtgattgctctaactcaccttatagcattaacccataagtgagtacttactgaactctcactgtatattagattaagggcttattttatatgcctggcacagttcttatttgttgttggaaaaatttttagagggcagtaattgaaatgtcatatactatagtctttgaggatggaaaagagctcagtaagtttctattaattggttatgccgttaagatcagcatctaaagagagctaggtcatggaaaaaatggcctaagtactggacttactatcagaagtactggacttaaattttaattcttatgatttgtgaacattgtagaaatatactaatgtctttgtgagtacagataaggaggaacagataagcttatgtgaaaactcggtttgtattgggtctatattatatacagggctattttattttatttatatttatttatttatttttgagacagagtctctccctgtcgcccaggctagagtgcagtggcgggatctcggatcactgcaacctctgcctcctgggttcaagccattctccatcctcctgcctcagcctccgaagtagctgggattacaggcacccacaccacgcccagctaatttttgtatttttagtagagacagggtttcaccatgttggccaggctggtcttgaactcctgacctcaggtgatccacccgccatggcttcccaaagtgttgggattacaggcgtaagccaccatgcctggcctattttatttatttatctatctatctattttttgaggtggaatttcgctcttgtttcccaggctggagtgcaatggcgccatcctggctcaccacaacctccacctcccaggttcaagcgattgtcctgcctcagcctcccgagtagctgggattacaggcatgcgtcaccatgcccggctaattttctgtgttttttttttttttttctttcagtagagatggggttttttcatgttgattaggctggtcttgaattctcgacctcaggtgatctacccgcctcagcctcccaaaatgctgggattacaggcgtgagccaccgtgcctggcctgttttattttattttaacaacaaatagaaggtggttattccaggagggtttcagaatactaccccagatttggtttctcctggtttgactacaggcagtgctggtgtctttctcttgatcatactcctttcctcatgggtgtgttcagtcctctgatgatagaagtgcttacctttatttaacctttccctctctcactgagtgttctctctttcctgaaatacttttcatttgttaaagcttttaagaaattcttatttgaacattcagtgttatgtaaaaaacaaattaactgctgcttaatatcatgtttggaggtgaaaaattttctaagccttttgcttagccatttattaccaaaggggtatcaagcttggatgtcaagctaggatgtaaattctagacctatacaattccttcaagggcggctgaaaagttgctttctaatccttttgctatattttggtggcaagctttgttttcacagtttaaagaggattgatggttttcctattgtctttccttcacaactttctgaagttcacttgcctgtttcattttcatgacttaaggtaccagcttcttctgcccctctcctagatccaccagcgccccccatgctccagaaaaattgagctacactgtgcctttccctgagctgtagaaagttccacctctaggtttccaggtgcttctgatcatcatttttcaatggattgagttgccaacatctacaaaatatattctaacaaagatgctcagaatgtctggtgttgacctatctatacaggtaaacttcaatgactctgcatttccctttcactgctgacctcatgccttttgcagaagtaatataggaaagaagagcattgaaatagggcagaagatctaattttctttttctttcttgagatggagtctcgctctgtcacccaggctggagtgcagaggctcggtcttggctcactgtaactccgcctccccggctcaggtgattctcctgcctcagccttccgagtagctgggattacaggcacccaccaccatgcccagctaatttttgtatttttagtagagacgaagctttgccctgttggccagtctggtctcgaactcctgacctcaggtgatccacctgcctcggcctcccaaagtgctgggattacaggtgtgagccactgcacccggccagaagatctaattttctaacttacaatgtactcgaatattgaaaggaatttcatatttttagaactaagccctcattggggttgggtacggttagggcaaggagaagcctgggaaggagttcttgggtagttaaattggaaaaagagagtctggcacattcagaatggctccatatttgttacccgcagactgtacataagaaatgagacctggctattaagagatatgatgcctgtgaaaaccctggatacacttgcaacttgctttttcccaagtgtttaggactctagtagggacacctccagttccttttggaaataagctaatatttggtttccaaaaatgtattgaagtagtcttaatgagacaaatagaaaatagaaatttgattgaatgttcttacattcttaaaaatgctactttttagcatttttaaaaataatgaagtacatgttggggtgggagtggagaagagactgtaatatttctgtatctgaagcctccataaggtcttaatccagccctccataggaacataagccataattgtcatctacaacttgttcctaaatttactcagctacagtttctcagagctattgagggaagtactacgatgctatttaaaaatactttcaaatccatccttatgatgatgaagttattatagttacaaatgcatgattcagcaataggcattaggaaaacatatccaccagacagcctgatgatcatggtgtatgacactttcttctgaatacatgagggttagatgaaagtggtgacaaacgattgagatatctagatccacattatttaccttttttttttttttgagacagagtctcgctctgttgcccaggctggattgcaatgacacacttggcttattgcaacctctgcctcctgggttcaagtgattctcctgcctcagcctcctgaatagctgggattacaggcatgcactaccacacctggctaattttgtatttttagcagacagggggtttcactctgttggccaggctggtctcgaactcctgacctcaggttatctgcccacctcagcctcccaaagtgctgggattacaggcatgagtcaccacgcccagccaaccacacttcttaaggctagataaatacatagctttaagtgtagagtaaatggtgtctcacttgcaaagaagaggatctcttggtcctctcctttccccaaccccaaataatggctttgctattggcaatgtaaggtgggtcggaaagtttgtgaagcaatggccaagggccttatggaacaggcatactgcaggggtggcgtggtcataagttgctgatgaagcagattctctgtcactcatgcctggtgaacaggcggggactcccttcttcaagagagacaggtgatgatagaagagtagacagccctgaacaaacctttcctcaggattgtcccttttggtattactcagacctgaataatacatggacatgttttatagggaattttttttcagctttttgggtgatctattattaggatcttattaaaatatgtataattttaaaactaggtatacacttcttatgcttatagttcttatgcaactatagagccaaaacaaattaataaatgaaatcaactatatctgagcagacctcaggatttgagagagagagagaaaaagaaatcaaatataactatgaaacccaataaaatttaaattattttaatgtgatagataatgtggttttgttgaaactaatcactagtgtggaacttaaagatagacacagtagaaccatgtatcctcagggtctggtctatgtttaaatctgttatttaaatgtattaatttcatgcaaagcatgcctgttagtgtaagtatgttgaacaaattgttattaatagctttgagggttcataagttcaggaaaataagatcttaacccaaactctgtagtaagcaatcatcgaatgtcagtttctatgaggtgtcatttgataattttttaaattgtttactcacttgaagatagggttagcattgtgttgatattaaaattggtactaaatggatatctaatccatttactaacagaatcagggacagaaccattcttctttgtgtgtttatgcctcaagtttctgcttataagctggtatgtgttagtatgtaaaaagccaaagtccccatagcacatctgctcacagcgtgaagatggcatcgaatcctatggctgagtagattgaactgcctcatgccatgcaacgactcagttttcccaacatttcctctatgtccagattgatccttgacttcatctggctttcactgggcatttgtgcaattgtaaacaccaatgaaaagccaggcagtgaaattgcatgatggttactgcttgtattaaaatgttaaattataatcgaagacagaaaattttttaaatcctagaactcttgtggactgcaagatatttatcctttgagacaaactgctttaaaattaaatttgggctccacggttctcaggaaaacttttctcatttgtggtctttcctttaagttccattaataattaaaaatgacaattgtgaggacttctgagtaatgagtgtttattatgctctaaaatttaagataagcactttacatatgtaatctcatgaaaaccttgtaattggtgtgaggtgagtgctttggctattttacatgtgaggacacatgacaccaagaaaaggtcaatcacatgacaagtaagtggcagcagcggaatttgaacctatatctttctcattgcaacgcccaagccttacacactaaagcctcttcttatgtctccatttcacattgtgaaaacctgtttaactgcaactaggaaccagtgctctacaattatcctcttacattcctgtcttttccattgggccagtgggtctcaatctttaatatgcatcagaatcacctggagtgctttttgaaaaatgcaggtcccagatgccactccgaatttactgaataagaaattgtctgtgcaaatgtccagggggcctgaatcttttaaaacaacctccctcaggtgattctgttgcaggtgtctgtctgatcacattttgtgtaaaactgcattagactgtggatgcctcaagaaccatgacttttgaaaaaatccatccttgtacctcaaggactcactcactaaatgcttgtcaaataagtaaaagaatgaatgaaggggcaattgtcaagaaagatgtgttcttgggaagaatacgcttttggccagacccttggaaaatttagagatgcatgtttaaatatcacttaatatactcggtctagactctccttcctggaggccaagcatggcaaagagccgatgagagggtggtagaaagtacaaatctatctataaaaaaaagaatgaaaatgctcctgaccatccaggatgagatagaataaatcttcccctgcgtttctaaaactaaagtcaattgaactagaaatatttaaatgcacttgaggcccagaaagagattcatttccttaatgttttgtggacttgacgttttctacttaaatcatagctctaaggtcaatatggatttgttaccggcaaggggtcccaatctacaccccaagagagggttcttagacctcacgtgagaaagagttctgggcaagtctataaagtgaaagcaagtttattagagaaggaaagaaacaaaagaatggctactccataggcagagcagtggcatgagctgctcaattgggtatacttgtagttatttcttgattatatgctaaataaggagtggataattcgtgagttttctggaaaaggggcagggatttcctttggaactgaggatccctaccctttttagactatatgaggtaacttccagatgttgccatggcatttgtaaactgttatagcgctggtgggaatgtcttttagcatgttaatgcattataattagcgtctaatgagcagtgaggatgaccacaggtcactttcatcaccatcttggttttggtgggatttggtaggtttctttactgcatcctgctttatcagcagggtctttgtgacctgtatgtgtcttgtgccaacgtcctgtctcatcctgggactaagaatgccttaacctcctgggaatgccacccagcaggtctcagtctcattttacccagtccctattcaagatggagttgctctggttcaaatgcctctgacagattcattcctgtatttagcaagaggttttttccttttttttttttttgagtacttactaagagtcttgtatttaataagacatagtcctttccctttctgagtttattttcagttagaaaattccgggagcagtaaaagagcaatgcaagtaaatggggggctagttgctcagaatctgcaaatgtaagagtgcatggaagggtttgtatagccaggggactggtgctggatgctgcaaattgaataagaattggaataaattggatagccaggtagcaaagagcagtggaacagacacttggcagtcagattgcttatggctggctggttcagttaacagtgaggacaggagcctagggtggtgggatatttgccttggggagcatggggaaataacattgcatagatgggttttctgacattaaatagagctttggatgttgtggaattttgaacatttcctttatctgaaagccggtgaagtgttgtcttaagatgtgacagcagtatgtaagcaaattgcaggggataagaaatcaagtcagggagacccattagagggctgcattctcagaacaaggagcactgaggaaaaaattaaaaaacaaacaaacaaaaacaaaaaaagaaggttgctttcggtagtccagatgtgaagggacagggtttagaccagggtggtggagtgcagaaagaaaagccagagggatgtggaaggacaagatggcatgaccacgaatggtggcaaaggcagcgtggaacaagcacatcagctgatgtggaatgtatgagaacaaaaacactcattttcccaccagaacccttagtaggagaaagtgccttaagcttacagtgaggattccaaattgaaattgctcagtagctgtttcaccctttgttactggcagagcagatggctctggaagagactaggactacttgttgcttctctttcccatctctcaccactccccaaccttatcccatagtgaggaccactcactagtgacatttctgcttgagacctacaaagcaagtcttgctaatgagacttaggcactttttcccaaagtgcaattcttggaaatctgtgggttttaagtagattacagggccacagttccgtggtcaaattagttcaaaaaatattgttagagaattaagcaagtttctttactgcaggatgtcccacagtctttaatatggtaatcaacattttgactatccaagagagggcgagcttagagcactccacaaactttttggatcatggagctctttttaaaagaagcaccttgagagaagaggatggcacagcatatatttaaagaaattagggactagaaagtttgtctcttgcactagaggttggtagtgtcatgcaaagaaacaagagttgggatttgggtccaggctttgccactaactggtgatcttgggtaagtcattgaatgtctctgagcctcagtttccccaggggttatgttcattctaaatgaactcttcagctcctttcaaaagtctgtcttcatattcaaaaaggaaaataatctgcaatttctctactctgcaataactctggtggtggaggcagtggtaggaaaagagagagtagagcggacagtgacaagaagatgaataagatacggtctgaacccctgcactgattagactgttgggcacaattacagtgctgggctgatgtaacaaagataaatgaagtttgtagtgtgttctcaagcttctctgactccaaccacatcatgtgatatacagcaactagttcagttgctaagcaacactaattcccaatggtcacttaatccatctagtctcagttctgctactaagaagctgtgaggccttggataaatcagttaaccctttgggtttgggttttcttcatccgtaaaataaggaagttgggttaggtctggaaccatttttaagttgacctgagtcttacctggaagaaggtaaaggaaacacagctattctcctctcacccaccccatgcagatgagccccaacattctgtcttcgcttttcttctcttccttcctcctggatcacttaatctgtctcagactttaattattgcatttatgtagttgaagttcaaatgtggatctctaagaccaacccttctcttgaatgccaaaatcaagatgtgtctacctgttttcattctaaggaaaccaaattacttatgtttcaaaccaaactcacaatctccccttacaaaccaattcctcctccacgttaaaaaaaaaataataataataacaggccaggtgtggtggctcacgcctgtaatcccaacattttgggaggccaaggaaggcagatcacttgaggtcaggagttcgagaacagcctggccaacatggtgaaagcccatctctactaaaagtacaaaaattagcagggtagggtggtgcgcacctgtaatctcagctactcaggaggctgaggcaggagaatcgcttgaacctgggaggcagaggttgcagtgaactaagactgcaccactgccctccagcctgggcaacagagtgagactccatctcaaaaataaatacataaataaataaataactatgtgctcctggtcacatgcagcccagactcccaggcttttcgactccctccttcttccctcctgtgctagtcattggtcaattctcaatcctctccacattattattattattttttgagacagagtcttgctctgttaccctggctgtagtgcagtggctcgatctcagctcactgcagcctaagccttccaggttcaagtaattctcatgtctcagcctcccaagtagctgagattacagtggatgtgtgccaccatgcccagctaatttttgtatttttagtagagacatgttttcatcatgttggccaaggtggtcttgaactcctggcctcaagtgatccacctgccttggcctcccaaagtgctgggattacaggcatgaaccaccgcacccagcccctctccacattattatatgtccacattatttatttccctttcctgatgtcattattgtacatcaggctttcattggtcatcctgccccaggtctctcctccaaagcaacccacatgccaggttcagcctcacagagcttctctgagcctgccatcccactctccaaagcctttcccggctctcttgtgctcactccttgctggcatttggagccctgcgttttcccacctcaatcatcttccagcttccagctttgtcttcgattacctgtgtgttcctgtcctcccaccaactatttattaatcttcttatcccataacttgcccattcaaattgttcccttttcttagacttttgtctgcattctttctatctgacaaaatcctagtgatatttcaagagtcagctcaaataatggctcctcactccaacaaatctttagggaacactcaactggcttgatttctccttcttttagcaccttgttctttcctttcttactaccttaccaacccagacatatatatctactatatcttgtaatatccattatatcttttttgaggccaaggaacatgtttccttcatctttctatcaactatagtacatattacatactaggaactcagaaacgtggttgaacaaattactattaaattattaagctattaaaacaataatctattgaaataatgtaataatcttaatctcctaacaaaatactaataaaatttaagtaaaataaataatctacaataatccaaaaatgagtcgaatcttggtttactctttggtgttttgtcattctactttcgtgtcaagcttagaaataattttctgaagaattttgattttgattttagttataactttgcgttagtaagaccatcagcctacggttaaggatatttgtctcacctgtgaaatctcactatctccatgaaaccgactggcttcgctacatcatgggaagggtgttatctttcctagttggagcatcatatgatgatgtggttgaaaacatgcactttggcataagacagacttggtttggaattctgactctgcccttgttggggctgcttggggaatttagttagcatctctaaatattagtttgctcatttgtaaatggaggatgtaattgcaggtgctggttctactcttggaaggattcaaaaattgtcatgcttacccgaggggctcctccaaacctgggcggggttttctgtatgccttatggacctacaaatgcaaaccacactgaccatgtaattttgggggcagattggaaagctgaaacagaagtgcagggactgttggtgcccatcttcctggcctggtggggaggaatgtaggaaagcagactcgtgcttctcatcttgaatcagacagacgacagacactccaggaaattccttttgtttggaaaagaaaaatgctctgccatcttctcttaccccagttttctacattcccagacttcctaaaagagtagaccactggggttccctgaaaagccagctaagtccagcacatacgatcagccccacagctcattttggttcacagacaataaggagagaggacttaacaattactgagtgctgagtacccactatgtggtaggaacattttgtacataattacaattatgcatcctcataactttatcacataagtatcatctctattttacaagtagacactaagcaacctcccaagattgtacagctagtaaatctgagtgaggaagtgaactcagatggtctacaaaacttttagcatttgtggccaggcatggtggcttacacctctaatcccagcactttgggaggctgaggtgggaggattgcttgaactcaggagttagagatcagcctgggctacaatggcaaaaccttgtctgtacaaaaaatacaaaagttagctgggcgtggtggcacacacctgcagtcccagctacttgggaggctgaggtaggaggatcacttgaaccaagaggtggaggttgcagtgagccatgatcacgccactgcactccagcctgtgtgacagaacaaggccctgtcttaaaaaacaaacaagcaaacaaacaaacaaagaaacttttagcatttgagaccttcagtgtgatcaaagcaaattagctttgtgaggtggggtttcctttagcttctacgtcagcaaaattgtgaaccaacaggattaaccttttgccgtcttagtactgcagggaactagctttcagaaaattgtttgggggccgggcatggtggcttgtgcctgtaatcccagcactttgggaggccaaggtgagtggattgcctgaggtcaggagtttgagaccagcctgggcaacatgtcgaaaccctgtctctactaaaaatataaaaactagccaggcatggtggcgagtgcctgtaatcccaggtacttaggaagctgaggcatgagaatcgcttgaacccgagaggcggaggttgcagtggctgagatcatggatcgtgccactgcactccagcctggatgatagaatgagactctgtctcaaaaaaaaaaaaatactgtttggggacatttgggtcctattttcttccctgatgctgatgtgatgctgatgatagtgagtgtttcaagaaccaggagaggctggttgttgtaaacattcctataaacgttggctcagagattgttaatgtaagtttttctggagcagtgaggtaagccccaagtagaaaagggcagaagtatagtaaaatttggttgttttgtcaactttgtaagacttcagctgcataccttgttaggaaactagaagccggaggaaagttgtgctgagatgtgtagcatttgttcttgctttgtgtggccattttattcccattaagtactgtatccttcactaaagaaatgggccattcagcagcccgcctgaacactgtggcactgtatattgttatagagagttatatccattcagagggacacaacagatcagaggaaaggaaagaggtaaccatgtatgcagacgtggaactgtccccaacatgtataatagacaagccaaggtgaaatgtatagcatactaatattcaactttttaaatttaaaaaactatgcatctcaaggcatggtggctcacatctataatcccagcactttgtgaggccgaggtgggaggatcgcttgattgcctgaagccaggagttggagaccagcttgggcaacatagcaagacctggtctctacaaaaaatacaaaaattagctgggcatggtggtgtgtgcctgtagtcctagctacttaaaaggccgaggcaaaaggatcacttgagcctgggggttcaaggctgtagtgagctatgatcactccactgcactccagcctgggcaacaaagcaacaccctgtctaaagaatacaacagcaaacaaacaaacaaaaaaccaaaaaccctcaaacactataaatccatagtttttatatatgtgtaaactatttctagaaaattactcaagtaactgataattagttatatctgaaggagggtgatggtgcggtggaaatgagactcacttttcatcgtgtggagcagtgattcccagtgtgttctgggattctggggagtccttgagaccctttcaggagatccatagatcaaaattattttaataataatactaagttgtttttattctcattctctcacgagtgtacggtggagttttccagaggctcagtgacatgtgccattgcagcacattgaatccagaagcagatacgataatccaactgtgtctggtcaagccaaatatttaagagattttaaaaaatgtaaaatgatgtcattcttctcatgttgaagataaagttatttttataacaattttgaagataaagttatttttataacaattattattcaacaaaattactattatttaatgagttaatacacaataaatttcaaattctcagttttaatttcttaactgggcaagtatcagtttaggtataaacaacatccctccgagatcctctgggatacatccaattcctttagtgcctggtgtgcttggaaggtttctgttgcacggcgctggacaggagtgccaaagaggaaagaaatgtgaatggaaccaaagtctataataaaatattgctttctctttttcccagcctccatcaagggagacctacaagttgcctggggttcagtgctctagaaagttccaaggtttgtggcttgaattattctaaagaagctgaaataattgaagagaagcagaggccagctgtttttgaggatcctgctccacagagaatgctctgcacccgttgatgtaagtaggtccagtcactgaggctctgttaccacatgagattgggagtggtgtggctcacattgaatccccccacttatcctgcctggtgtcacagggaaagttgaacacttggtttctgtgcctagagctgctatcagatgagaagacaatgtatttgactatataggaaatctgttgtggactgtcaattaactcattttcacttccgttgatttggttgtgaccagccctagattagagggcagttttagagtgtcctggcctagaatgtcttcctcagttttgagtaagccagggtacctggcctctttctgggacttctctgcccttacaggacccactggaatctgtagggttccagcgtcaatctgtgttttctaatattacaatttgtttttattttttatttttaacaattattttataagttgttttcttctgggaataatttgaggtgatttagagtaaaacaatacttaatagaagcactacaaccaggataatagtacaagagtcctgtaattatattttgggggaattaaatgtaagaaaacctagcccagggatactgattgcagattcatacagcgcttagctgggactcaaggcaaaaagagaagcatgctatattgcagaacttttattaggaaaccatttccacatatcaaggtggtctgctgcaggtgaattttggtctgaatgggacttctcagttcatcctttggcaggctaaaatcccactgaggatctagaactgtagcaatttctagattgtgtctgagaccttcccagcctggagctccatatctccttcatgcctgtgcctctagcatctagcacagggtctgacacatggaggatgctcagcgagagccctcagattcaacttaggctttactgtattctgcacagcttctggaatgagtgctgaatgaatgaatgagtgagtaaataatgtgaatgattattgggataagggtggggctaaagtggtattttcttacattccccagaagatttcttctccctgtcaagatgctttttggattatctcttcctttctgtttattatctagggaatgcctcgctgctctttagcttcctggtggtaataattcacttcatagtttaatgtcttaggtttccttcttcccacagattgccttcaaatttgccttttcttcctccctccctttcttccttcctccctcctttccttcctcttttccttccttatgatagactcttggtatgggggagttaagaggtcttaacttgagccttcacagtgtatattttagaatgaataattcattgctatgcttcaccaccaaccagaaattattacagggtttctctattacatgagatttttcaaacattatttatctatgatttgttccgctttacctttcagaaactatcactgtggtcttttcttcccctgccactggagttttttttttttttttattttttttgttaacagggttttgctctgtcacccaggctggagagtagcagtgtaatcttgtgcaatcttggctcactgcaacctctgcctccaggctcaaattatcctcccacctcagcctcccaagtagctggaaatacaggcacacgccaccactttgtattttttgtggagatgagggtctcactatgttgtccaggctggtcttgaactcctgagctcaagcaatcctaccaccgtgccctcctaaagtgctgggattacaggcgtgagccaccatgcctgggcaccaatcttaaacaatgatttccataaattactattagaatattcaaggcatatgctatatatagccttgtcctgtgaagcctgattttggtgcactcctccacccccactgtcatattatccctggaactatctcaaacaccttggctcctccaggaagagtccttctggtcaccaattcaggctcattctctttctgggaacgatcagactgagtttttcaatctgtttagagatttactaagttttagtttcttgtgtttcttattaatagggagaccaaataattcagcatccaatctggagaactaatgaggacaggagaagttattaataggtaatctggaacataaggcatactctggatgggtagccacctgcttttcaaagatcttgcctctttaatgggtactgagagcagaagggtaacaacagcctttggagattgaactcctgaattctgttcagttgtctggggctagggaactctgacacagatattgtagcactgagtatttctaacaagctgtggggggcctgtcccctagtggatgttagagttcccagagggagaagtcaagaccactgcagcaccgacggccccgtgcttatgagatccagtggtggtacccctagtcagtcttcctccaccctctccttcctcttttcccttctcctccccttcttcccttcccctccccactccccagtctgctcccttctctttttgatccttattctctccctctcccttccaacgcccccacctcggctccctcttgttttgttttctctttccatcgccttctctttctccattttcttctctccctttgccattcacagtgtcttcctttccttttctcttctaaaatggctcttccaaaatcactggagggcagctcaaaagagcaactctgccaatattaatttctccttgaaagggaaaagcctgcatttccaattatgacttagtcttcattagttagaaactgtcttttttccacagcaagtggcctaggaatggtgtgtttctcgcaagtctctggtcggaggtcctggttttatgtattctgctgtgcacgctttctagggcatgaagacagggcaggctgggccattggaggtgggggtggcagtgaggatggcggtctggctgtgacaggaacacacctggaaatgtctgcttgtcacagaatggattgcagctgaagtaaacaaaactctgtgatcatgttgctgcataaacctcgatgtctgaaatttgggacgatggctgtgccaggattattttagaaaggcaagctcaggatcagtgttcaactaaccagagaatctacagctgctgtggatgtccctccaccagagagagagagagcctagcagaaactgaggagagaagctcttcaggccgctcaatggctcacgtctgtagtcccagcactttgggaggccgaggaggaaggatcacttgagccaggttggagagcagcttgggcaacgtggcaagaccttgtctgtacaaaaaaaaaaaaaaaaaatgccaggtatgatggggcctgcctgtcgttccagctactggggaggctgaggctgaagaatggtttgagcctgggaggttgaggcatggtaagccctgttcacatcattgcactccagcctgggtgacagaatgagaccctgtctcaaaaaataaataaataaataaataaaagttcttcaaagaggaatggaggggaggaaccaagggctgacatggacaattctaggtaaacagggctccagaaacattctgaaaagcataagctgagagcagacttcagtaacatgaagaaagaactggggaggaggggagcggtctagagaataacatgctcttatttgttcatgggattcataaactgtggacaatatttttatttaccaaaaaggtaacaaagtttattgagcacctgtgatgtgtaagggactctggaagtattttgaagggatacaaatataaataagagataactccagccttggtttctcatgggctaataggaaaggaaaggaatgaaaaagagaattacactcaagggaggtaagcatgagtcaggacctgacaaatggttgaaggaaggaaaggacacttgtagctgggatgttcaaggaacacatgctttgaatatggagggtgtggagcttgacctaagccttgggggctaggaaggaatttgatatgcagaaggaaagatgtgggtcattatccttggagctgacctaggcagtgaggacccagagtgatctggcgaacacagactgtgctgggcatgtggagtggaggggaggtaggaaacggctgtctacttggggcagaggattgggttgaggagtggtggaagataatactggaaaagaggacacttgccagaatgtggaaagccctgactgttggctgggctaagacatctggacagtctggacaccattttagggattcagcacgtgcgttttacgtgctctagcagggaaggatgactttactgttagagatgctcatttctgatgtccctctcgctgagtgtgagtgcagtcctgaataagattaaaggtgtcataactataaaccatttggaaaatgtaaaaatatatatgcatgagacacttggagcaaggattttctaaggtctcaatttcttgtttttgctttgattgttatagcttataagagtagatttaagggttagccacaaactctattactgtcagagcagatttgggctgctaggtagaatcttattttcactgtttcaattctcatttctttgtaattttttgtattatactacatattttagccccaaattgtgggctgcaggaacaacgttaagtatttcttgctggctgtgttcactgaattgcttcctgagcctgaacttgctcaaacttcctgttttttgggcacagaactcctggtttctccatttatgatggatgagaggggtttggagtctctcttctagactttcatagcatgtttcaggatttctcactgatatctaattgtcttataagagacattaaatttttttcacactcaaaaatccatgatcccctctaggtaaggtctgagtctctgtcaaacattttcgcttttgtgtaacatttagcacagtatttatcaggcattattttgtttgggggtagaggaggtaggcggcggtgggcagttgggaccccccgtacttatgttctgctccaatattgtcttgtagaaaatggtctaaaatagatctttactatctgaaatatcatctagtgagagcattggcatcatcaaagagcttgttaaaaatgcaggatctaggacctcagactaaatcagaatctgtaatttgattggcctttgagacttgctggtctccagatgtaatgtatacacatcacctagggatcatgttaaatgcaggttctgacactcaattacttaggtaatgatcaaacaaccagatcttgaagttttctaacatcaaagcagcaacttcctttctagagttgtcaccggacaccttccctcctgagagtgacacttctgcacagaacagatggtttatgacaggagttggaaaactgtggcctgctggtggcatccagcctgccacctctttttgcaaagaaagttttattagatcacagtgacgctcatgtatttacatattgtctgtggcttcttttgtgctgtactgactgagttgaatagttgtaaaggagactgcatgacacacaaagccaaaagtatttactatctgggactttatagaaaacagtttgctgaccctggcttatgtgagtgactctaaatatttccttaaagaatcaaatagctatactctcttgccccagtggcaaacttcgcaagcccatctgaatccatggctattcatagagaagctgaaccataaaccgtcattcccaggcaacccacaaagggtcttgagcctaaaattggtctggataatggtaccacagagcctgtgactttccaaaatccatttaatatgtccctcctcaaagaaatggttataattttatataattttattgagctgactccctcctccacaaattgatttcattaattgttcaacataagtctgactttccccaagaattggaatcaaaaggaaattctgagtaaaaatatatctgggaccaaggaaataaaaagacttgtacaagttggttataatacacattttactcctaaaaaggtagcgttggttttggccccactctggcctattcactgggcagactgtatgggcttggagtccttcttttaattcccttggagaatcccaccacctgtctcttatttgggtttttgaattgtgaaatgatggcacacacaagctccaagaatagctgcgtgtgtacaccaagcatacagagacagaaggcagagacttaggccaaggatcaggaaatacagaagagaagctggcatggaggcatctgctccacaaacacagtgaagccggaggttgtaggaggagggaggtgggacccgtgtaatcactggaggatgtgggcactctgctctgctctgcaagtcttggagatcttggttttctttccagaaccaaatttcagaggaggtgggtgtaggagaattgagattctttttcctcatccagagttatctggggccaaagaggtttagggtgagattctgtgggaatctttgctgtagctccagaagctttattcttacttagttccttttccctttgtctgcatagtccaggacactaaaagggtttgtgaagagtccgggtagacataagtggagagtcttctccttaattctagagtctatcttgacttcaaccaattaaagattgtaggagtgaattagagccccttatgttaaaccttgtttcttggaagccctcctggaattccctaagatgcagagctgattagcttagctggtcagaatgtgaaggttttaaagccaaagttattatttgggctgatattttagacattctcttcattggaagaagtacaatgtattcattcattcattcattcactcattcattcattcactcagtgaatatccattgaaaacctattgtgtaacaggatgcaggaaaaacctgaactggagagtcagacacatctgagttcaaattctagtttcttgatctgtgagctaactaacacctcattcagctttccatccaggtgacataaccaccccagcttccttccctgaatctgcaggttaggttggacatttacttctctgcttttgcagcaacttctacaccgtctgttctcataccacattatatggaaattatccatttgcagtctgtctctctctccagattgtaaagtcattgatagaaaagagcttcatttcacatacttttttacccttagtgcctgacatatattagaaattagacatatgtttattgcactgaaaagaacctaattgtgtggcttaatttccctaaagcatgtgtttctcatctgtaaaatgaaggtgataaaaacatgccttgtgagttttttgtgaagatcaaatcagataatgtttgaatgagtgttttataaatatgaagtgcttgcaaatactaatagttgttctaatatctggattgattgcgattcagtagaaaacaagcagaagagaatcaagaaagtatgtggaggtgtcctagtccactttttctaaactgaataactgaacgagggggatctggagacttcagcttctgaggcacagtttgggtgtgtggggttgacacgggacagaggaccctttccagcaagctcgatgtctctgccattagattatatactcataacagtggggatatgttttattaatttcattgcccctttattcaccctaagcataactaagaaagtgaatatgtaataattggatttcaatgggcattgcaaggccaaagtctcctgccccccaaatctcccagtatccctggcctgtatgtatcttttcttgagaacctgaataatggaaccattagattgaaaacatagattggggagaagagagtgagttgaatttgaaaaatgttgagtgtgacatctggcagtcaaatatgcagggctgactcatttcacttttcatgtgtctgttttagctcacaaactagacatagagctcattgtacagattccatagtgcttaatgcactggctaccccttaaaaaacctatagagcttgctggagggggtcccctatctcatgtcaactcaataaataaatatgtatggatttgttgcaaaatattggattttcttttccatgtaagtaacaaagaactgctgcagatatttgagcagagttgtaaaatgatatgaattttgctgtgagaaaacttgatcttgctgtaatgtatgaaaaagactagaagggaaagaagttgagggtggagagtcttgttaggagggccaggataagagcaggtgggcggtgaaggaggctgcggcagaaccatgccaagcagaatggagtggaggggacttgactacacaagaggctggtgactcagctttctgcctagtattagtcctagagaagtctccctgctctgggaagaccggtgtctagcctcatgtggcttcggaacaaaagccagtgattgcatccaagggaagataatgagaaacacacaggccgtgaaaatcaggaaagcaccactaatctttttgttgcaaaactttacttttcaaagtcctggggaagggtgcagaagcctcagcacgtggacttgagccctgtactcggaaatgcaaaatggaaaccccctaatagatcacagaactgaaaatatccaagagtctgagaggctgggggaggacaggagtgagaaaaggaagggggctggagtactgatcagaagataggagaaagcaacagtgcaaaatgcggggaagggctgagtagacattcaatttaatctggacttttattttgctacaaactaattttatgaagactttttttgcccaaatattaaatatttattaattaaagatattaaaacaggaaaaactgctttcattcaataaaaactctagtaattcttcctaactccagaattgtatgtatatgttttacctattaaataatcagaaatagaaacaaattagacaagatacgtgaataaccttaaaaattttccataatatttgacttaggaactttagttttatgaatgtactctaaggaataccctgaaaaggacaaaatctatacacaaagattatacacaaatttattatagaattatgtactaaaaaaccctaaatgaccagccttgagtatatggttaaatatataagttataagcctttgggttttgagagttttagataacacaagaaggtatttatgatattatattaaatcaagataaaagtcacaatgtaatataaagatatacaatcaatgatgtaaaagtacatagaagaaaactctaatgaaaatatgccaaattgttaactatgatgacttttaggttacggaattatgggaccatttttgtttccttatgcaattgaaaagtcataaatgagcatgcatcactttttcatcagaaaaataaatgagttacatatttttttaaagggaagatattgtaaaggaatatttgttgacagaatgcattaaaaaacctaggtaatttccttatttccctgggattctgagaatttttttactcttgggaatatctttccttgctcttagttattttcatatgttagtcttgttcctggggataaaaaatggtaagtttgctatttgagctctgctcgaacatatggagtagttctctcttacatatacagaattggtttgacattcaaggtctttcagagtcaaatcccaattgatcttcccaatttcacttcccttagtttcctacgtatattttctacacaaatcaaaaaccttttcagacttcctatttcttgtacatgtccataacctcccctttctgaaactctacttgttcctggggacagaatgttcacagagcatgcgttaaacatcataaagtggatcgggagcctcttttaacataaggctgtgtgattttgacaaggcagttagctcaccctctagatctcagatggaacacctgtaaaacaagggactatgttgtagatggttgccaacattccttgcagtgctacagctgagtgaaccttgttccctcatggccctaggtgtgctcattcatctgcctgcaccttgtctcaggttcccactgcatcttaagtggcccctgcccaccccactgtcaaatttgacccttcctagctcatgtaccatttcctttataatgccgtcctcagtgattctgattaacagggacttgcatgtatgattttttttttttttttgccactcatacaggctgccttttatcaaaactatggcacatgtcaatgatataacagataatatcatttgaataaaaagtcaactccatggggaggaaaaaaacctacttaagtgagctttagagataaaggtgaacacttctctaaatcctttgtttatggtctgttaatcaataaagtccaatctgtgatccccagatacataagaggagaaggatatgtagactatttatgatatttctgaaaatcctaagaaaaatcacacacaggcccacagtaagtctatgtttctttattttgagtcagaggatttcttttttccatttttaaccagtataaattggtttaaacttcatctcttgaataactcctaacatgaccagagcccttttcttccttcaaatttccacaaatgcattgctccccttttttcttttcaccacccatatgtccatgttggacaaaaaccatgaaggaaggaggttgagaaactgtgtaccaaaacgccaagagcatctttgccatgttccagcacaaagggcactgttagaaggaggtgatcagagtcatcttctgactcaacctcacaagaggagtcaggttagatgaacagttcccaactatttactctcctctctctctcattctctctctcgcattctttttccttctgttttttattagcagcacaaaagattttgaagtgcttaagggcagaaagcttgccttgttcatctatgcagctcttgctgggaccaagtatggtgctaaacacacggtagacattcagtaagtatttgtggaatgaatgaatgagcaaatgaaggacaggtgtggcttcgatggcaatgggtcagtgtgaactccggaaagagtatgcagagttatagtcaaagtggatgatggatcattgctctgacttactagttatgtaatcttgagtaaacccctttatcactgtgggattaatttccccagctgttgaaaaaatgtgtgtgggggaaagggggagggttagtgattaaaaatgtgttccataaggagcatgtatgtaaaatcatctggcacatggttgcagctgaatagctggcatttatgtagcactttattttttctgcattttttaaaaattaaaaacctgaaaattgttaagttgtactaacctcaaatctcaccacttaagaattatataaaataagaagtaaaatttctcttcccagccaacttttctaattccccagagataactacttttcttaacatttaagtttgatgtttatccttcttgacatttttctaaagtttcatatatatatattttttataattatttaatatatatgtaaactcatgaatatatatatgcaaactcataaatatatatatatatatctccatgcaactttggatggagatatatatacacacatatcattttcaaaatggaactattcatatatgtctatctatctatctatctatcaatcatctatctatctccaagtcaattatttttgaatgatggcagaatcatttgttgtttgaatataccatattttgctgccagtcacccagttccagtgttttgtgattacaaacaataatgcaagggtcttccttgtacatctatatctgtaagcttttgtacagtacttactttagtaataaggcacttgaacacttattattctctttactcataaccaccacaagaagaaaggacagaaattgttattctgtgttacagatgaagaaaaaggagagctaaagggggcaactggcttggccagagacccacacagatagactatggcagaccccgtgtttcccagtggcttgaatttttgtccagagctctttttgtcacactgcaataaagcacaagtcaacaggaaacctttacaaccaggcagagaagcttggatcagggagcaggcagtcacactaaattgtagaaaagaacaaacacacacacacaaacaagaagctcgaggccaaacacatttaaaggatattatttggaagaaatgcactggtggtgagtgagggaccagcataagaagagctgtcctttaactagttaccatgttttatgcaatgttctcaatatgatatccaaatgattaggatggcttagattgcataaatgaggcaggagaatgacaaaggacacccccacaaccccaacaatgacactacccctctccctcaagggaagtgtctagggaaggccagtcagcatctggttaggcttggccttaattgtcatcttcctcaattatctgcaggaaatattagctgttattaaaatttgcagatgcagtcaattgtcagagtcactggagaccaagaattattactcagagcattggagagaggctggaaagaaaaagaacaattcagcctggaaaaatgtaaactaatggataaagtgaggtagggggagtgggggaagaatttagagggataaggccagtagtagggtgaggcagtgaagcactcccctggagtacaaactttaaggggatgccaaaaactcagctatcaagataaataaaattttaattcaatattgaaaaaaatcaagttggcaaatgacagcctgtgggccagcagcctgtttgtaaatacaattttattggaaagccactggaagtaaaggtactgtggcaggggagatccaggctaattctttgatgtgaaaaccctggttaggcttgtgaatgttagacacattagaccttagcttgtgattgatgggaaaaatatttctgaagaaaaggaataatggatctgactgcttttatgggtttcctggggttactgtaacaagtgatcacaaactgggtggcttaggacaacagatattgattgtctcacagttctggaggctagaagtccaagatcaaggtgtcagcagggccatattccctctgaaggttctagagaagcatctttctttgacatctaatttttgatggctcttgcaatcactccaatctctgccttgatgtttacatactcttcattgtgtatgtctctctgtctttgtgtccgtctctctgtctttgtgtccacatctttttctcctttcccttagatcctagtcattgggtgacatcccaatccagataacctcattttaacttggtaacatctgcaaagaccctagtttcaaataagattacattcctaagattacctatgaatataaccttaactctttggttccttatttggaaatagggtctttcttggacacgattcaacccaccacactgttaaagatatctttctaggacacaattcaacccaccatgctattaaataggtagggatgtggagagatttgacttaggcgcagcagaaacattatccattcacagaaatctggcagcggagtcaggggcagactcctgacatgatttaaatacctaagaaaatatttaaggaaaagtactttagttcctttggggcgcaaatattataacctttgctcatgtcacatggtgacaaatacatgcaggatttttaagggtagttctgatttcaaacatgcaaacaatagtgactggaaatatttttactaaattttatattccttttgttactattttattcttctcaatgtaagcaacttattaaattattactataaccgaatataacaacttttagatatttttcacaaaaattcaccaaccggggaaaaacgttgtttgtaaaatgcagtattctgatttttaatttatgaaatatggctgttatgcttctgaagcctgttcaaaggctatgaaacaatggagatgatcgaataccattagaattttaagtaagttgaaatgtaaatgggaaacaaatctattttctcccattttccaaacacatcacagtcaagtccaaaaatttgaatgttacatggctttttagtgaacacttttgttataaacatgcgtgtgtaggtatacttaattgtatcagcaacaataacattagttattttaattattgtatcaccaatcctaatcactaatcaataaatgtatactcattaattaataattcaataataatggctaatatttatgaagtgcttatgtgctagacagaagaattgatccctttatgtgaattatttaatttatgaatacagtaattcttccttagaattggtattctgtttatttgggcttgaaatgttctacatattatttttcctgcttttggtcaggagtcaccagcccatttaaagagagacagctgtatctttctgtaactgggctattttctctatgagtgcattaatgttaacagagaagcatggccagcaagggctgctagcacaaagtgaagggccctgctgtgatgaacagagaaattgggaactttatcattgctgctatagacattatcaactaataacacgtttttaaaattcctgaaaagagtttgctttctaaaggttaaaaaggtttaagttctccctgtaattttgtttatatagatgatttgctttgtaaacatttgttttgtttgtttgtttgtttttgagacagagtcttgctctgtcgcccaggctggagtgcagtggcacgatctcagctcactgcgacctctgcctcctgggttcaagcaattctcctgtctcagcctccccagtagctgggattacaggcatgcgccaccatgcctggctaatttttgtatttttagtagagacgcttcaccatgttggccaggctggtctggaactcctgacctcgtgatctgcctgccttggcctcccaaggtactgggattacaggcgtgagccattgtgcctggcacagctctccattttaaggctttgcaattgattgaatcaggctcgttcaggttacctaggatgatcttcctcacgtaaagtcaacccatgatggattctaatcggtctacagaacttcttcacagcaacacctagatcagtgtttcagtaactggggtctacagcctagccaagctgacagatcagaagagccatgacaacaatgataaaagaaactgttgccctctagtttcctcctgggcattaataatttagctaatataaacccaatactttggatacgattgactgaattcagctttgctgatgcctctgatttcagttgctttaaccaaatgcaaacccctagtgcttctgttgtctgttgatacccagaggctcatgtgatctggaagggtaagtggcttctctctgatttgccagaagaaagtggttatgttagctgaggagccagaacactaatttcagtgatttgactctggtacaagtggtgggaggcccgagggtgggatgctgctttttcctttggccctggtaaacattttatgcttgttatggtggttgaaatgctgtatttaaccttttcttcatgtgccaggaggttcacagtaaatgaggggtgtggtctgtgaagggaaaagtggctgtggcttggcctctttgtaccctgtcagcacacatacacgcatggtctgtaacatgtgaacctatcacgtgggaactcattactgagctgtgagggctgcccctgaggagctgggggcagtggtcgggagaggagaaggtcgtggggcacagtagaaatggcaccggactgggaatgagttcctactgccaacttctcatcagctcagttcctacagtcctctacttactccttcgttgcccaggacatttatttaatgccaggctaagcacaaggcttccaggattcagcagtgaacaagacagatgctacccttgcccttttggagctgactgttgagcaattttaataaatagcaatgatcatatcattagcatttcccgagggttaccatgtcccaaatgtgtgagttatgatagaggaagatggggttattctaggtgaaggccgagaagcttctgggaggcagtgactaataactactgccttttttactttggagtcatgtcctttctgtgactcccctttccttctcctctgtgttctctcccttggccttgtcaggctgcccctggctttggttctctcttcctagtctcctttctaattagcctttctagcccaataaattaagctcttgtctggaatctctagctccctgatggacacctcctcgtgaaagccctcagtatctcaaagacacaacatctgtgaagaactcaactcttccattgtattcttccttccctttcctgtctctgataatgttatcacaattcgttttgtcatttaggtcaaaaacatagtcatctttgacctaccttccctatcatacctaccagtcactcagccctaccctgtctattcccatgatgtctctcatctctccctccatttgtttgtcactctcaatggtcagactgtcttggtcacttttctgggcaattgctatagtttaatgcttgcagttttccctcaactttagttcatcctctttagagctaccagagcacatactcttgatacatcttcttgcgtcactctgttgctcaaaagccttcagtggaccactgctgggctctgcagccaggtgcttgtgttctaaacctggctctgtcacttaccagcttggtaactggaaaattattaaccattctgtgcttctattatttcatctataaaacaattgtgtctgtctcatagagttgtgatggggattaaatgagtcaacatgtaagtagtgcttataaaaaaaagtctgacataattttagcattccgtccaggtagatgctctggctcctggtcctccacattttccctcttgtgccttgactttttgcgtttctaacttttccctgttatatttctgaatagttttttgcaaagtagtgcacaaattagtgcctggatctacacaaactacatcatacatgatgctcttgtctttcttcatattcttttaaagactatatgttatatatttaaacacatatgcatatttatactaaactaagtatatatatgtatacacatatatacatatagattcatgtatactttattgtttcttcattaattgaccaaatgttttgatggtgcctctaaataagtagtacttaggcaaatctccactcatagtattgtggtaaaagattaacttttgaaaactgcccagagaacagctttctatcacattaatcatgagatacagttttgttttttccaatgttctggcggccattctcatgaggatggactgtgctgactgatctcagtgaaagttaccagtgggcatgttctcggggcctctctgtccccatgaagcatccccactcagagacatggaaaacctgtgagatgagctcatacaaatgatttctgggatgaaaatagcaatgtgagatagtgccaggaacttcaggttcacagagtataattaagatttttcctgaatcattgcacactgtaatttcatcacatcattatgtacgttctttaattatgggctctaattgcccattttagttctgtaattagacataaagacattaattttgtaggtataacaatcagctcaaatgtacttccaaattctttcccctacctttcaagaaaaattaatacatcattaaaaagtttgtgcacagaagagcctcttagagggcagcttttgggagccagttggtgttctcaaaggtggggccccagttcagaaagggtatgggaacatcaccctcttatattccccataaactgtgttgcttcatgtcagatagaccctactgggtccagggcagggaagagctccattaattcacatagggccgatttctcttaatcgtaaaattttaaggtgacatcaatgcttaatgagtcatgggagaggaatgaactaatgttagggcaactctggctttttatttctcttgtgtcctattaaaagatgagaagatgaagcattttaggctcatggggtttgtatttttcatttgtgcatgggtagctgattatcatctgtcattatgtgcgtgtgtgtgtgtgtatgtgtgtgtgtgtgagagagagagagagagagagagagagagagagagagacagcaagagagagccagaatctgttttttaagaactctcaatgattcagatctccattttttcccttcacctacagcactttagttaaaacaaaacaaaacaaaacaaaacaaaaaaaccgacgagttccagcatttccccccatggaccatgctatgtccatttcagccgacacattttacatgtgtatctatttgcctccacttacaaatccaaggaaaaaaaggcagtgtggtagagtagaaagagccttagtaagggtcctggcccctttttcttccctttttacacatactctctctctaggtaatctctctattctcgtccttaaaggccgtttatatgcttttgtccctccaatttatatctctagctcaggcctcatctctgagctccagattagataacaatcagaatgcttatttctacttggatgatttacagataatttaagcttgctgtgttcaaagctgggtttactttcctccaaatgtattgcttttctattcttcccaaactcaataaatggcaaaaccatccattcaagtactcaagctgagtcatccttgattattctttcgacatatgagccatcagcaaggcttgtttcaccctccagaacctatcttcaatctgtctgtttccctctccctcaattcaggctgcaatcccaatgcagagtgttcagtaaagcaaaaggggtggtctgctctcaccaaaagcaatttgcatgctcttaattcacggatggattacctagcacttaacacagacccagatatatttggtactcagtaatatacactgaagaatgaatgagtgagttaatgaattaatgaatgaataacagaaactctacctcctaggacttaataatacagagcaggattaacctgctttcataagtaatcagtgaacctggtaaagtgtgacatttatcttttggtgaaagcagctcaccccttttgctttactgtttcaccagcaatctctgttttatttttacctgttcttttggacacacaatttagtcagaaatttgcacctgagtcttacttctgccacctgctagtataaagatcaactggtgcagagcactatacagagattatttattgtttgctacaatgttggatatagagtttcctctctaggacctctttaactgggagggataaaaatggcacaactaaattttcatgatttggataagaagagaaacattgggtgggcatggtggctcatgcctgtaatcccagcactttgaggggctgaggcgggtggatcacctgaggtcaggaatttgagaccagcctggccaacatagggaaaccctgtctctactaaaagtacaaaaaaaaactagccgggcatggtggtgggcacctgtaatttcaactactagggagcctgaggcaggagaatgcttggactcgggaggtggaggttgcagtgagtcaagatcatgctattgcgttccagcctggccaacaagagtgaaactccatctcaaaaaaaaaaagtagaggagaaacattgcttcattcagaaagtgcttcctaagagattcccatggtccagggatgatgctggatggtggagatgtagaataatgtagagattacagggtagcagggatggggatgcacttgtaagccaatgagtagaagaaaggggttctggcggtggcacagcagcaaccacaggacgaaagcaactgcagaggacatgtgctcaattctgtgaggggacgatggcgggacatggaagggtcctgatcatgtcgatgttgatggcaagtctaccagtcaaaaggaacaggtgagcaagggcctgtagcctggaggtggcttggggctttcaaggaaccactttgttagacctgctaaagcatgtgtttcaaagagaatgtacccagctcgaagacttgcacattgtaggcacttagctaatatctgtttaataaatggataagtgaatataagtggcagaaagacagctgagttgctgggacaggtgagatcatggaaggccttgtgtatcatgctaggatcagtaatgggaagtagagaagtggtcaatgaagttttatttaagcagaaaagagaccctattgggttgagttttagaaaaatccctgtggctgcagaaaagaggatggtttggtgggaatgagactgaagtcatgagcacagacaggaagggtaactgaacattttgagagagctgaaagtgtagacaggaagggtgttcaaagattgttggagttcagagagaagtcctcggcctggcctagcagtgtagacagccaaagacagacaagtagtggaatgtcacattctagtaactgattgaaatgtgtgtgtgtttataggcaagtgagttgaaaactgtgtgtgtgcttaagttggaatgtacatgtgcatatatgaaggtgatttgaagtgtgtgtgtgtgtgaatgcagtgtgtggtagtgcattggaacgtgtgtgtgtttacatgagtgcaagtggacagtcaagaatggtgtctaacacttacctatttggtctaaaggctcataaaacttctgccttactcctggtcacatgctccaagtcacaagcagaaatgggcagaacagctcggccttgtaattccagcctgactcacactaagctgtttccaaccaagtgaattaatttctccctccttatgcccacacagcactttcctctacaacaattgttacaccactgttgttattagtttatataatactttaccccctgctaggctgtgaaatcctgaaggaggagaaaatatgttatgtatcatttatctccagagtctagctcattcaggcacatgctaagttctcagctaatgtctgttgaacgaactaaaagaataggcacacttttatagcttattaagctcaaacatagactccaaagcaaaatctcatcattgcctcttgcacattttggaaacaacagcaataatagttaccatttcttgagaacctactgtgtgcaaggcacagtgccaaatactttgggggagccagtgattttatgtgcaatgtttttaaaagtccctggagttaaaaaattcctaggaggtaaatattatccaccccttccacagatgacggaagtgaggctcactaaggataagtaacctgccaagttgcacagctagaaagtagagagctgaaacaggaactcaaagcctgggctccgaacaagcacgccctgcagacttcccttccctgtgatgccccatgctcagttttctattttctttatagtggaaaagtcaatgcccttcctatgagaaataaaagggtttaaagtggtcaaaaggaaacagtgtttcctctgtgctcacggggttttggccaatcacagagggtgaaccaaaaacaatattatgcgagatgtggccagtttgggttgattccagctttggatgctgtgagctggatcccaggagcacacgaggaaaaacgataatagcatacttgagatccaagcccatgttggggtgaggatagaggttttgctttcctcaaactattgcatttttcttttcctccctaagggtccgggcaggatgcttcctaccccagggggccacttccaaatgctgctctgtcacttggtggaaaacttcaaatttactgagggtttactgtggaagacttagtcaagagggcaagtaccctatatctccagattgctttgcacatgttggcattcaattaatattttatttgtcaatggatttaatagctgattacagtttgcaatgctctactcatggataaatcagttgatcttctcaggcttcttatgccttttcccagtggatttttcattgtcgtggataaccccaggaaaaagaggaggcacagaggaacctctcttacatgaataggtggggggatgcaagggaggacttctagcttttcttctcctgacttttctacattccccaggttgagtgactcgtctccatcttttaatttagcatcttggaactgaacagcacctcccaagcaaccctcacctgggaaccaggcagaaacaagtgaaaatgtggaggaggtgtttatgaatttttgggcagtcagtgatttcagggatccaggaagttattctactttctaaaccacttccccacctgctggatatgaatgtaactccctcagctttcagctttcacatctactttatatacaattctatactgcctctcccattttatattatttatacctatgtgctgggacttgagcaaaggtttttttattttttatttttttttattttttaaactttgtacaagggaagccataaagaatttgggcaacttgtagggaagagggagagtgaatttggtgaaaccgaaagtcagtaaaggtcacatgggagatgctgagctctgcagacagatctttcaacgtgaagcagcagtgaagaacatggctctggagtcaaactggctgagctaaagagttgactttgttacctactagttacctattggtagttacctactagttatctactagttacctacctctgttacctactagtgtgaccttgggcaggttaagtaactttttgaacttagtttactgatacataaattagtgataataataattcctgtctcataaagttaccgtgaagattaagtgaggcaatgtatgtggagtgttaaaacagtgcatgacgcagagtaaacacgttaaaatgagctgttattaatatttggttgctaaaggtctgtcctacctgatgtgtctatctctttcaaatacatcctctgggatttagcactagaaacagtcttgcagaaagttagagcagggaggaaccctcaagatcatctaatccaaagtcctcttttgtaaatgaggaatcactggcccaaggtcacggagtaagtcactgagtaatccctctggtctgttgcccagaatgcattccttcccacaaatctggccatgcttgtcctggagggcagatagactagccttccccaggtaaccttgtatcttcccgtaaaaccgtctgtcctcacaaagtggcacaatttctaaggctccctcgggcaacaaagtaggttgtgttcagagaccagtgaggtggagaaactgcccagccactgaactccaggctggacttggtgagtggcactcagtggagtcagagtcctggagacctgcacatcagggagagggtcctgaagaggtcttgaaataatttgacatggaaagagaaaatacaaattttcagagacacagataagcatcatatttttaagccaagaactcagttatatttttgacagtgtggtgtctacagtactaaggcatgttcttacaagagtgacactcactagctcttttttttttttaactttcattttagtttcagtggtatctgtgcaggtttgtcatatagtaatggggatttggtgtacaggtaattttgtcacctgggtaataagcacagtacccaataggttttttttttctgattctctccctcctcccactgaccatcctcaagtaggccccagagtctgttgttccactcctagcatccgtgtgttttccttgtttagctcctacttgtaagtgagaacatgcgatatttggttttctgttcctgtgttaatttgcttaggataatgagatcatgttctctgacatggaaagagaaacacacaaattttcagagacacagatgaccatcatatttttaagtcaagaactcagttatatttttgacagtatggagcctccagctccatccatgttgctgcaaaggacatgatctcattcttttttatggctgcatagtattccatggtgtgtatgtactatgttttctttattcagtctaccattaatgggcatttagtttgattccatgtctttgctattgtggatagtgctgcaatgaacatgtgtctttatggtagaacaatttatagccctttgggtatatacccaataataagattgctgggtcaaacggtaattctccttttagttctttgaggaatcaccacactgccttccacaatcgctgaactaatttgcactcccaccagcagattatgtgtttttttttcaaccttgccaacatctgttttgttttgttttttaataatagctattctgatgagtatgagatggtatcttattatggttttgatttgcagtgctctaatgattagtgatgtcaagtatttttttcgtatgcctgttggccgcatgtacgtctttttttgaaaatgtctgttcatgttctttgcccactttttaatggggttgtttggtttttccttgtaaatttgttcaagttccttatagattctggacattaagtctttgtctgttgttcccctcctagtttgcaaatattttctcccattctgtaggttatttactgttaatagtatcttttgctgtgcaaaatctttttaattagttctcattggtaaatttctgtttttgttgcaattgcttttggtgtcttcatcacgaaatctttgccaagtcctatgtccagaatggtatttcctaggttatcttccaaagtttctatggttttaggttttacatttaagtctttaatccatcttgagttgattttcgtatgtggtatataagataggggtccagtttcaatcttctgcatatggctagccagctatctcaatatcatttattgaatggagagtccttttcctagtgcttggttttgttgattttgttgaagatcagatggttgtaagtatgcagcattatttctgggctctctattcaattacattggtctatatgtctgtttctgtatcagcaccatgctgttttggttactacagccctgcagtatactttgaagttaagtcgagtaacatgatacctcctggtttgttcagctcatttctttcttgttgaagacttcagttcaatggccttagtcatctttggaatggcatttaggattcagttcaatgaccttagtcatctttagaatggcatttaggaaaatggactcctttaccaaaaaggtgaaaaagaaaataatataacccaaccttaaagactatttttttaaggggagagggagaggactttattcataatcctatcagctgaacacattccagtcttcttctataaaaacacaattttttataaagttgaaattatggagctaagacatttctaagaagacttaatttcagttagggacttgctgattatctttgggtaagtcagtctggcttcttttctgggaggagtgttgtgtccatagagacgtgaacatcgacgtctgcttgtttttgtgaggaagcatggtagaatgaagagaacgctgtctggagccaaaagatccagattgaaaccagactttaagcctgggctctgcttccccttttgctggatgaccttgagacagtgtcttaaccttcctggatctgttttttagcttttataaaatagatcactggcatatcagcttgttaacgtgcactgtactcagccttgacacacgggagaaaggttttcagggacagggtgaagatttcatctatatgacctttgactgcagtttggttggttgggttacacaatctacaattgtagattgattgagagtcttttgaaatatgtttttctttatgctagttacccaggtcctctcctctctggtgacagtggtaatttaccagggcattgtgtcctcttttcattaattaggccactcaggcctacccgaatcccaagagaccatttatggatgatgaaggtggtaagatagggttgtagtatgttggacctgctgtcacaaactaccatatgcagggtgacttataattaagagaaatttatttttcacaattctggaggctgggaaatcctagatcaaagtgccagcagatttggtgtctggttaggcctgcttgctggttcagagataactgttttcttgctgtgttgtcacatggcagaagggggaagagaactctctggggtccttagtataagggtactagtcttatccacaagagctctgtcctcatgatctaatcaccccacaaaggcttcaactccaaatgccatgactttgggggtcagatttaaacataggaattttaaggggacacaaacattcagtttatagcagagagtgtatgttcacagcacaggaaggttcacagcagaagggagccattaattgtgtatagactttcatgtctctgctctgattcattgtataatctgcttcattcaaaggaagaaggtaaattttgggtccctcttggaacatttaggtgactcccaccaaatacaggatagctttgagttataacctcatgaagtccctggtcatgtctgtgtgtttgctttggaccagacccaagggcctgtcctgtgtcctgcccgggctcctcccacctctttcctggagaaacccacagtgagagtggagggtgggggtccatgtttatcttggtgggcctcactggagctggacactgctgctctgtttgcccaggcttaagtatgcatctccttttttaactggccagggactttcccccagtcacttggcctgccattgatttgggacttttctgcctaaaacttctaggatgataaggagagcactattacccactgcctgcccaaacctagtagcaacagcagctagtcatgaccagcattcatggctgcaagtactgtcaggccaggggcctgaccagtgaactggaactttacttctctcccaagtgggattagagttcactgagtaggtggccctattaaaatatgtatagcctgtgctaaagttaattctaggggtgagaatgaggcagctctgcttgctggctagcccaggctggcaaagaataatgggtgcaagcaagaataccagagccagattgggttcaaattccaattctattgcttcctaggtatactgacttaggcaagtacttctatttctctgtgtctcagtttcttcacttgtgaaattttcatattttaaggattttgaagatatcattttgaggcttaaaacaatgtatttacttatgtttaacacatgtaattactgatgtttaacttttttggacttataaaaatgacaatgtttagcagagtgctttaaaaacatgacccaactatgtgctttctataagaagcccctttgaatataatgatatagccatgttcaaagaaaaaggatgaagaaagctatattatataaacattaattgaatgaaagcaggagtagctatattaatacatgtgaaaatcctctacaagatattagcaaattgaatacaaccatgtacaaaaagaattacacatcatatcaaattagatttattctaagtatgcaagactggttcaatattttaaaatcatgtgattatattaattgatgcagaaaattatttgacaaaattcaatattcattcatgacttaaaaaaaactctcggcaagttaggaatagaataattacctcaattaaaaagtttacagctaacatcatactttgtgaagaaggacttaatgttttctccctaagacagggatcaaggcaagaatatctgctcctgccactcctattcaacatagcactggaaattctagccattatagaaaagaaattaaaggcatacagattggaaaggaggaaataaaactatatttgaagatgacatgattgcctacagaaaaaaataccaacacatttacaaaaaactctcctagaactaataaattctcctagaactaataagagaccacaggatacaggctcaacaagctcaccggatacaaaaatcaattgtacgtctacatacctataatgaacatgctgacaccagaataagaaatatagtgctaattttaatcactcaaaaataataagtgtaagtacatttaaatatattatatttgtaatgtatattatgtaagtttaaatataataaaacatacataggacttgtatgttgaaggctacaaagactgatgaaagaaataaaaagatctaaataaacagagagacatactatgttcatggaagtacttgacatagtaaagacacaatatactaaagatggcaattctccccaaatcaatatatacatttaatataattcctacccaaatctcagcaacgttgttttttttaaaatagatagaaagaggattattctaaaattttgactaaaggctgaggaaccataatagctagaacaatttttaaaacaaactaagtgggaggaatcagtctaccctatttgaagacttattatatagctgtagtaatcaaggctgtgtggtattgacagaagtacagacatagatcaaaggaatgcaataaagaattcagaaatagacccacacaaatacacctctgtgttgccttatttttcagaaaagctctagactcagagtcaggaagcccaggtttaggtccttggccttgcacaagtgagttcttccagttataaaactttgattttctgggtctaaatcacttaaacttttctatctcagttttcttttctgtttaagggaacatccttccttcttccttccaagcctctatttgacaaggatgataaaatatctttcttttttaagcataaggaattctacaaaccaaaagaattattacattggttgatgtgaataacacatctcaacaccatgaggccctgactccgcaaacctgaagtcaaatgccaagaactttcatgaaatttattaatttattttttcttcccaactaacaccactaattctttatttggactccttaaggagcatgaagaatgggactcattcttgcagaactgtttttagagtttgctactgctcaaagctcagagtttggccatgtgaacttctgagaagtctgtgaataagagattttacagcaatttaacagtggttcaaagatagatgctctgtctcccagtagaatacttgtgcttgtgtgggcagaaaattcttaaaatgattcaaaccaaccacccaaagaggattatatgtaaattcatattgtgtgggtcctgggagctttgaaaaccacatacacagagcacagaaacacacaaacacacccaccaacacacacacacacacacacacacacacacacacacacacacatacatgaatagcacccttagcaccattgctaccttatgtgattcctaccatgaaaatctgcactttaaaaagcaggaaaatttgaaatctacagatgtggataacactcaaacttgaattgggacacaagaacttttccctctcgggctagtgagatagtacggcctagtggctacaagggtgaattctggagataaattttctgggtttaaatcacaattttacctttcattaactgtgatttggggcatgttttttaactattgggtgccttggtttgctcttcagtatgattggaataattatatacaaaatatacaatatctatctcacagtattgctgtgaggatttgaagaaatcatttatactaagcttttatgcatgaaatatatttagaacagagtgaagcagaagtatctgtgttctttcatagaatgtatgaatgaaatgggacagtgggtggtcactgccttaaaatactataccatatattttgtcatggtgctattgcaacaatactcctaacattatgtatttctttatttgagtcactgaaatacaaatatgttctgagaagagtcagcagagtgatttacaaactagctttttagactgggaggcacactggagatccaggttgggggcctgggtctaatctcagggctctacttttttaacaaggtatataattgtggacaagtcaactggtgttcctgaacctcattcttacctgcacactgaggaggctgaactgggtgatctcggaggtcctttctggctgtaatatcctaagagactggatgctccctaggcaaggaaactgttttatcgacgtacatactgtggaagcttactaatgcaagtctttattaagtagtttttctgtagcagccgcttttctaagccctttgacctatttaacccttgcagcaatcctatgaagaaggatttatcattatccccattttacagataaggaaactgaagcagacagaggctaagtgatttgcccaaagaccatatggctattaagtggcagggctgagattcataatccagtaagttgaggtacgcacaccaggcattctattcagcatcagacagtgctaggcaaagaataaccgaagaagagggactgtgttctattcactttgttttccccacagatctgtaagagtgcctggagctttttataggttctgaatagatatttatcattcttgcagtggcttgatcttggctcactgcaacctccgcctcttgggttcaagcgattctcctactttatcctctggagtagctgggattacaggtgcccggcaccatgcccagctaatttttgtattttttttttaaattttattttaagttctgggatacatgtgcagaacgtgcaggtttgttgcataggcatgcatgtgccatagtggtttgctgcacttatcaacccaccatctaggttttaagccctgcatgcattaggtatttgtcctaatgctctccttccccttgccccccaccccgtgacaggccctggtgtgtgatatcccactccctgtgtccatgtgtcctcattgttcaactcccacttatgagtgagaatatgcggtgtttggttttgtgttcctgtgttagtttgctgagaatgatggcttccagcttcatccatgcccctgcaaaggatatgaactcattgttttttatggctgcgtagtattccatggtatatatgtgccacattttctttatccagtctatcattgatggacatttgggttgattccaagtttttgctattgtaaatagtgctgcagtaaacatacgtgtgcatgtgcctttatagtagaatgatttataatcctttcgatatatacccagtaatgggaattttttgtatttttagtagaaatggggttttgccatattggccaggctggtcttgaactcctaacctcaggttatctgcccgccttggcctcccaaagtactgggattacaggcatgagccaccacgcctggcctatcattcttgtttttgttgacgacaaaaagttgttggattaaattgctttcagccttctgggttgctaagtgttgcagagttggctgtgttaaatttcattttcttttgttaatgttacccacacaaggtgaaggttttggttggggctcaggagggctcctgactggggagaaagtgggaagatccagagcaaggagaagacaatgaggctgccaggctttgggctgctctggcctctctgctgcctgccactgtcttcatgtccagcctcgctgtgtttctataaaggtggagttgtacctctctgatttcccctaaaggtggcctggatgtggtagaagaaagaggaggcaaggcattacctgcacctggaatactgatgagtggtttgctctttcttgtatgcccagtggcctggtcttaagcctaggaataaatgtagtcaccatggccataaaacggccccactgctgggcttgctttccctggcctctgtgagttccatgctcattatatggactccccatagcaacgttgagagcttgcttatcatttccagaagtgaccatggcagattaggaatatatgtttattttgaagtgggtttttttctttttaaattttacacattgttggatgaacataacatcaagggctgctttcccaactgtcttaggaaaagagctctaatgttggagccagtagccctgcgtgttctcatcccagttttgattataactgactataatactgtaatgacactctgacctgtgttcttttatgactgagatttccggtggggatggaggaggtgtctgatgtacagaaccagggggatgctgcctgagccaggttggggacggagagaagaggctgaatttcaagacactgacatcatcagtactggccctagtgcagttacatacctggtcctgcaacaaataatgagtatttagggcacatatgccaagcattaatggggtcacgaggtggatcgttcttttgggtgcccttttatatgccgctgcaatcagagaagcaggacaccatgtaggtgagcatctgctgttttcacagctatttgaagctgcaacccagtgtactaaaagtttccaggctgactcagtccaaaggtgactgttttggaaactttgaatgagattggttcggcaacttcagtgttatgtaagtaaaaggatgagagcagttgaaagagccactgctctggacatgttctaagtggcttctaccaatgggaatacgtgagcgtgcattaagaatgtagcaagtgagactgacatgtctccagtacaagcacagcacgtacaaggacacaacttcatgtttggttcaatgtgagacttctctgtggctcctttctcttcatttgcacagatatctgtaccacccagtataagtggtatactaatcgtagctagcctgtgctgcatgcctgctattttctgggtaattcccatgtgttttacacaggttaactctacttcctctcatggaaactacttgttgtcgaaataatggttatattttaccaatatggcaactgaggcacagagatgttaagtgtttcagcccaggtaaataagtggttcaatcaggatttgaactcaagcagcacctaccttggagcctaagctcttaaccacagcacttcactgaacctataaaatacatgtttattggttttaagctctgctagttgaagaacatttatacatgtctctattaaaaattcacttgtgtgtgggttatgaattcacccataaagaagactgccatgacgcacggaggagggatgtctgtggagcttcaggagacgtgataatccacaaactgtcctgggttgaacttagctattattctgccaggaaactgtctggagaaggtccaggaaagtaccaagaagtaaagaagtacctgagagtgctcagctcccagtgtgtcccttggcacctgccagtcacagcttggttggttctggctgtggggccggccttcctgagacccttgtcagggggctcataccctggtgctctgttttcactgatgatgaagcctcagagcccttggtcccctcatgatgttctgcgggaaaattgtgcacacttttgttgtgtgacttaggttctttaactcaggctggttgacatggagcttggttccagtgatcagtgctggctttagatatgatttgcgcatctctgcaggctagtgtcacgctgcactgtggatctggactgtggctcgggtgccagttgcaacatagtcttcaaggatagccaatggcgagacaacagcactctgtcatgcagctgaagttatggaaacacatgatgtcacatgaatgtctattgtgtgaggtgatggttaatacctgtgtttccaagaaagtgccaggtactggctagtcctcaccacaaccctatatgtggtaagggcagttattaagcttattttataactgaagaagcattagcaccaacagagaagataacttgcccaaggtcatacatgaaaaccaatggagaaattaaggtttcaacctaggcagttaaggcctaaagtctgtgctcgtgaccaccaagctatctcaccggtcccctgtcaggctttttaggggataaagtacagagtaaagtagaactttcctcaaagactgtcttattggtctcacttattttttttttttgttagtctttgggtttactttaaattgatgggctgctggaaaaaatgtaaattgtcatttaaaaagaaggcatcaaggttttgtcttaggttaatttttcaaagggcttaccaagaacacagaagacagaatgccagctgggtgccgtggctcacgcctgtaatcccagcactttgggaggccaaggcaggcagatcacttgaggccaggagtttgagaccagcctggccaacatggtgaaaccccatctctacttaaaaaaaaaaaaaaaaaaaaaaaaatagccaggtgtggtggtgcatgcctgtaaccctagctgcttgggaggctgagttgggaggatcccttgaacctgggaggcagagattgcagtaagctgagatcatgacactgcactatagcctgggtgacaaggcaagacaatgtcaaaaaaaaaaaaaaagacagaatgctgagcaattaagaactactttatttagtcctgtcttatcatttgtggcctttgtgtattgagcacgtcctgagagctgggcactttctaaatactttacatgtgtttcctcactttatgcttgtaactgctttctggaagcaggactgtgattattcccattttatggaagaaggaaatgaggttcacgaatattacataatctgctcatagtcacaaagctagcaggtggtggggctggcgtagaaacccaggcagtctggctccagagtccatgttcttaattaaccactcctctagggtggaaagctcactggttttggggttaggacactcatgccagctctgccaaaatgtcaacatgtgtgacactggccaagtcatatctcctcttgggttctcattcatttatttactgattcattcagcagctctttccagcatacctaccatgtgctaggcattgttagtactagatacataacgggtgataagatagatttcctcgatgcttctgtagagtttacagatggattttcagtttcctcatctgtaaaagatgagaattaaaatatatgaatgtttctcatcctttacaaaacccacacatctttttataaacataaaaagttatgccttccctaatgttatttgaaatttctgagtaaataaccatgactaaaaataaattaaagcaattataatagtgagtaatttttgtttgaatcacacaagccctcatgcctgttcctgaggagattctcctgcgctggccctctgagccctgggcattctttcggttaagaatcatgggaatagtattcttgctctaaaaacggacctacagttatattttttaattgctttgaaatgctggagttgtgtctatttaatttgtattcattctttcaccaagttttggctcttgggtttgtatggcagataaaactttactcattccttttactgaaaacctctttcccccccagggtatagtcgtgagtcacgagctctctcaccaactgacatttccccatcctgtttttggagggaacaacaaaggacatgcctggtgcctgacacagcgggcccacaggctgctcctttagtggccttgtgagttcgatgagcttttggctctaggaagtgcctgggctgctattactctgtgaatctcatgagattcacagacctcttaggagcaccagccaggttgtaaagatctcttgaggactgggctctggaaggactggccactccaacaccctgccttgcccttaattgtgcattgcctggtgttctttaattgcctcacaaatgcccttgaaaactctcagggatggcctcactgtgcatgacaaaggaatgcaacctactgtctggttgtactgaaggagcagagagcaggattctcataatgattgttcaaagaatttatttatgtggagtcctaaaaggagtgataggtgtgaaaaaatgcaatttaattcaacaaacattgatgcaaacgctgtgcttggtgacagcaatatagagatgaataagaccagtgtctagtgacgtgctggcaagtacaactggctctgtggggaggcggtaccctgatttgtaccttttgctgatttccattgagtaaatactcttgccatgtttgatttcaagctaccaacatggtgtcaattggctcacagaagccctgaaaatttaataatcagctcttatgagcctgtacaaactgaacttgcattctgctaccaatatctacttttgaagttgtcagtttagattggaagatacataaattgccatgcagtctgataaagacagaaatagaagtatgtagaaggtagaaaggtatcacaagggacactggttcaattctgcggtgggcatatccacatggtaggctgcaaacaaagacaggcaaggcctgtaggtgggtggagacagagaccttcagagcatcctcagtgccttcaggtcatggggaaatgccttggcctgccttaaggtcccgtgcagtctagcctcaccttacctggctgaacattctcccacatttactgacacaaaccttctgctctagtgagtccagcccagtgttttcagtcttgagagcacatccctgccttggctgacacccacctcctcccaccaccgtttagaaagtgccttccctccacctccacttccctgctctactcatccttctttatgaatgcttcctacaacagctccaagccacagaaagcactgacagaaagggaaagattgatcacgtgaattccattaaagtgaagaatttatgtttatcaaaagtcactgttaagaaaggaaaaggcaaacaatggagtcagaggaactgtttggaacacttatgaccaatcgaggatttgtattcaggacatgtaaggacctctcacaaatcaataagaaaaagacaagaaaagggaaaagagaccagaacaggaacttcacaagaatggatatccaaatgacatgaaaagaagcttgccttcactccacctttacctcagtcactagggaaatgtgatgccactacatacccactaaagtggctgaaaccaaatagacttgtattaccaagcattgttgaagacttgaaagagtgggaaataatgtatactgctggtaagaatcagacatggtataacctatttgaaaaacagtttggcaatatttattaaaatggaacatttgcatactctatgacccagcatataatctgaccatactctatgacccagcatatgatatgacctagctataatccaatgaaaatgtatatccatatgtttacctaaaagtatgtactggaatgttgatacagcatcattagttatggctccaaattggaaatacccagacattcatcaatagtaaaaaataaattataggctatttgtgaagtggaatctttttaaactatgaaaaagcatgacatactgctccatagaactacatggatgaacctcacaaacctaattttgagcgaaaggggccaaacaaaaaagaaaacaaatggagttattatatttatataaagttcaaaaacagacaaaactatatgaggttagacatcagtttacctgatacctttggggaagagcaagaggttagtgactgggagggctgtgttgggggcttctgagttgctgaggttgttatatttctggccctgagtggtgataacacaggtatgttcactttgtcattgagctttacgtgtgcaatttgtgtacttttctgtatatcttacatttttattagctaaaaagcaaaggagcttctgcatgaaagtattagaactagtgctggaaagtgagggagtgtttctgaaccgcctttgtgtttttcttttgacgttttctatctttatgagtttattcatcaaacaaagacattttgagagtgacaatgtgccgggaactattttaggtactgagagtgaaacaaaatagaaacaactgctgctttcatggagcttacattctcaaggggacagcaaggtccttgaacagacaaatgtataacaagtcaggggataagaaatactctgaagtggtattaggggataagaggataaagaataatggagctgggctgagggtttgggcactgactaggtgttgttttatatggggtggtcgaagaggacctctgaccaggcagccttggagccagagcctggatgaagtgagtaagggaggctgacaggtatgtgaagaacattccaggcagagagggcagtaagcgcaaaggccctgcattgggatccttggcctccgaagcaggaaactcagcatcattcttggcttctctctctcttcttcaccttcatatttgaatcaaatcatgatccatttgatttcatagctaaatctctcatagtttgcaacctctgcctcccaggctcaagcgcttcttctgcctcagcctctcgagtagctgggattacaggaacatgccagcacacccagctaatttttgtatttttagtagagatggggtttcaccatgttggccagggtggtctcaaactcctggtttgtctccatcccctccattgttgcctatttgggtaatagtttcttaactctgccttcccgtttccatttttttctcttctgattttaagtcactgactggatatgccactcctctgcctaaaagccctccagtggctctgctttgctctcagagtaaatgccctacaaggttttggttttcctcactggtctcatatctttctgctttgccgtacactccgtgcttgagccacaccaaggcgattccatgtttcccacattccctctgaggaaaggccctttagtctctgggtctttctttatgagggtgcatctacctgtggagttctttcccccacctcctcattttatgttttgcacatcccttagatctcagcttagatgttgtttagtctgggaagctgttcctgtcccttaaaccttggccaagtccctgtgttctcctatcgttgccccgtcatatcattttgtcattgatttttcttttttccagcctcccatgaaactctaagacacatgtgggaagtaactatgttacttattcatagtgcttagcaaagtttctgatgtttagaaatttctcaataaatattaaaaaaagaatgcatgttgagctacatttctgtgatatgtcaaaaatctagaagtatttttattagtaactgatagaaatatctttcagtcaaaacattattcatcaactcacaaaaaagcagactagctttgtgtttgagttttgtttttgtttttcctggtaattttgtgttccataatttctcagacacagaaacattatctttcaaattttaataccactttatttgactcaatcacatgtaaaatttttgttcagagaattttgaatgtatccgtgaaaaatattcaaatgtcgaaatccaaatgtcttgctttggctgtggagcaatatcattactgtgacttagttctagaatttagacttaattgctaaaaaaaaaattttgaattcaactttattaattttgtcaagtttttgatgtgctaggtggacattctttccaatttattacagtcccttcatatagacacaaaagcttttaacacgatgggaaagctggtgagacaagtcagaaaataaagcagctcttttttctttttttgttgagacagagactagttctgttgcccaggctagagtgcagtggtgcgatcttggctcactgtagcctccacctcccaggttcaagcaattcttgtgtctcaggcaccgaagtagctgggattacaggcagtagccaccacgccggctaatttttgtatttttagtagagatggggtttcaccatgttggccaggctggtctcaaacttctgagctcaagttatccacctgcctcggcctcccaaagtgctgggattacaggcatgagccaccgtccccagccagaaagccgtgatttttaagctgatgctttgtaccatcagtgtgaggagtgatgctattgatccctgctgggcatgagcattagaagaaagttgagcagggacttaaaatggggatgtggagcactgtgccaatgtctttcaaatgggcttttaaaaattattttaacaggattatttggagaatcataaatgtttgagcctcagatacactcatagctaccatttactgagtacttgctatatgccaggcactataaaaactgctttaaatagacaatcttgtttaattgtaataacagtcatatgggattgttaccatcattcccatttaacagatgagaaaattaaggctcagagaggttgagtggtttgccctaggtcatatagcaggtcaaagctaatgctgaactatgaacacagttcctctgactgaaaatgtgcaaggctactaacgactaggtcctactggcttatgtattgaaaagtagatcaatgaactcattctggagctaaatgttgtctattactgtggattatccatgacataacccactcattatattccagtattggttaaaaggtaggagagaagtgatagtaaatttcatgggttcggcatatcttctgaccgacagaagacatggaaatgttttaaatgtttcttttcctttttttttttttttaaaaaaaaaacaaaaacaaataacactaaaagtttcaccaaacaagggttcataaaaggatcgtctctaaattgtgagtgtggtgagtgtgcatgctctgagcaaaaagagcctcccctttctgagggaattgtgaccctggaaaggaggagagcaggaacagctggccatctccccctgaaaggccagggcccccctctcaggcctctgggctccagctctggctgagggtgtgtttgtttggctgtgactctttcccttctgtttccctctcggtgtcatatttcatgagctccaggctcctgcctgtttcagcttgagcctctggctttcatcgctgcctccttggccaaagctgagtgagggcttgaaaaaaacactgattggatgcacatttagtggaggcgattgctggaccagcacctcaccaatccccagccccaaatgcaactgacagctacaccctcctggctggagcctaaaccagaggcccctttcctccctcctctccacccatacccttgctgtttgcccaactcttcctgctgaaatcagaggcagcttgtgcctcccagccttcttccctgcccccgctgtctgcccagccaccagcttcccctgggctggaaagacccccgcttgtctgactgtgctaatcctctgtttgaggttccagaacttcccaattgttgggcctgggttcctgtacttagctgcccagagtccacttctctgctgagatttctattatacaagagattcaaccagaaccctctccgtgggcctccatgcctctttttgctgaattccccattctcattctgcgtagggggtgggagttggtgggggtacatgcaccttgagtctaatcatgatcttactggggagagctcagtaggaaagtaataacatattcatttaaacaactttttctctgttaatctttttattgaacttcatagaattaatggataaagactattggttagagtgcgtctttcttagacgagatggagttagagacacttaaaaaaaaataaggcttgccactgcaggagtgcacagagcacctcaactattggagcccaggtgatctcttcagtgggtcataaaatctgagtcataagaacctcatggcaaagaaaacttgaaactgcaaagtctcacatagtcatttgctgaaaactgaagtgtcccaacaaatgtcgggttagacctactttgagaaacacattccaatatgagatcgaacctgacaattttgcatgctaacctggactgtttcttattagagcaaaggggatgggctttttgtttgctttgggtgtatttgtttttgctgccaaagtgttgtaatgtcatgattatttaatatgtatgaggcctagttgtgtactagggagagtcaaaatacatttactacgaaatgagaccagctccagttgtttaaacagctccagttgtaataataagagctaagacttgatgccaagcactgtttctaggtattaacttattgagtcctcatgattactcctcacgatggtcctcttggaaaggatcctatttttctatgagaaaactgaggcttgagtgttaaggaacttgcttaaactcacacagttagtaaaggtcagcttttagatttaaagccaggcaccctcattctagagcttgtggctcaaccactgagctatactgcttgttagggttgttggtggagtgatagtagaagtagatgggctagagtttttcatttctaaccctaaaaatctatagctattaacactgaagacagcaactgccaattaaataaatcctcaacaagactgagaagagaatctgctaatctgttggtcactaatgtctgcagtttatctgcttatctatctgcagcaaaactgtcttagaacactttgctgaatgaaattgaacatggaaagctgactgaagggaaaacagagaaggtgggatagttgagaagggagtaagatcagacaaatggcaatgctctgtgggatactgcgtccaagcagaacagtcaagggattcagttctcagaagctgttcaggtagacgccctccttaccagagtcggttagcctcttgctgtatgcacacttgaggctgctcaaagcgcgagggaggcatggtctgtgctcattccaggcatccaaagcaccagggaggcatgatctgtgttcattccaggtgtcttatctcgctgccacttccaggcctgcacattataggcaatatatttactacactctcccttctcctttattttttacttctggatgtgccatggtgaatatttttcaggcaaatggggggaatcgtttccatttcaagctgggttgaaatctagcactatgattagggttgtgctaacctctgcctgcctctcccctaactcctgctgtgtataggtgggcatggctcaagcatcctcagggctgggagaaggttgcatcaggaatgtttctaccaacagaagctggccagtgggaggcttgtaggcactttgtaatttaccattctcttcttgaactatgcagcagtctgaggggctgtacagaggattcttcccagtcattcccattgctagctttggaggccttctcccacaattcatagcctcctagtaatgtgctttattcctaaagcccagtccttggggtttttgaagaaaaggaaggcagatcagcatgccttccctagtctgaaattaccctgtttgactcatatagtgtggagctttataatctgtaatccaaaattgggattgtctatactataaggatattctctgggacttacgcttatttaaccaaggttgtctgcaatgggaagctcccatgacgggagattcaggagaaggctggcataggcttctaacttacaccagtgctccctgatgcctgtgaatgtggggagagggcaaggagattttgagaacagataagagacatttgaatttctcaatgccttctgaaaacctgtgtgcagagttgaattgaaacttaccttgttagcattataaatctgaacactttccttgctaggccacaatgacttaataatatcgtctaaaggatctatgttcactctgcttgcactgatttacatctcctttttccaattctgcgtagtctctggacacttctacttccaaatatcacctgtttaaggaatagcacttaccttgtcttgtactgtgaacccaaatttctgtgaaaaatggactgtagcagttgttccagtttgagattgctgagtgtcatgaacattggttttgaagagtatggagtgttgttggaggacagctcgaggctaaatgtggttgtgcaatatggtttctacacactgtccagcaggtggttggatacagttatgccattctctgcttcttgtttgttgtcttcaaaacatgaaaaaataaaagcaaaatatagaatatcttcgatgaattcagcttctcttaatctcacttttatctcctttctgtaagacttagtctattatttccgaatatgccatcaattttatccaagtgaaaattcagttttagttttaaaacacacagatataaaaataagatgcaaagttctacagtaaaattcttagacataaacactgagatttttggattcacaaataaatagtttagataacactcagaaagatcttttcctcctaattagcttctattgtcacctcttcctaagttgatatgtagctcaatttgttaggaaatggatttcccatgcatgccctaactataatcataagcatctgcaactctgttttcaaacaaagcatctgcaactctattttcaaacaaaggtagttactcatgagagagcttctttaaactgcaatggaccacacagaggtggatgtctagcacatttacagtgaccatgatcctatctctcaccatttcttcttttcaatattgcaggattctaaccatatgactttcccacccctcacctgatgtttggcgacaatatcctggcttctactggcaacaccatcttttgctatttgggtaaatgtgctccttatcagccttggatcctacccaggctcagggccttgtgagtgcaatgtctattgggctattgtggaccccatgactatgacccagacactcagttttgtatgccacatcccttttcagggagctaggtcaaggacatcatgcaactcatttgcatgccatattgaagattcacacttgggggaatattttattctcaggcagctaagaccttgaaactccagcatagtatggagagtgctgagccttcagcatttttagcacatgatttcagccttggaaatctgatgtacttgcagacctctacacaagaggaggtatttgcatagaaacatgagcatgctggggctgggtcatccttttactacacatgttaaagcatacattgtaaccaaaattttggaagcaatgagaatctcttactctactattgggatttggggtcacagcaagactttggaagaggtattactgagaagatgcctctatgaggtgttactcaaagtcattaattacttacagggctctacgagaatggcatagttcatgtcttaaaaccgcttatagctactggttgagacagaaaaggaaactatgtgctgtgtgattaaaccggtgttttcttttttgcattctgtcttctacatgtgttgtggtgaaatgaaactttaccacattcagcagtgctcaggagaaacattgagcagtgttggtatatatttcaatggcattcaaaaccctgataaataatttaggacttggggagatctgggtatgggaagaagaactgaaaaaaaaaaaaagcagcggtaatttactaatatatgtttgtaggttaaaacactttttatagctgaccgtagtagatagaatcattctttacaactattaagtggtattaaataggttgacccaactcccttggaaaagtgattaattttgtcttccagacctgacttctgaagctcaaagttggtttaaacagttcagctgtagtagaggatagcctgatatcctagttttgccacagtatctgggccagatggcatttgcagtaaatggaaggtaacaatgatcagctctggtatttacgtgtgtaggtgatatggtcatgtgggagtgttcataggcttacaagccatatggctcttggccataaactattgctaggtaaggctttgactctgtatcagtatgaataaagtagctctttcagctcatgagtatccatttgctttgtgtcaggctgatgtcaagttaacctgcttttgcactctggtgaacagacacatagattattaaaactgaaagtgatctaaaaagttatctaaaccaatatcctacatgaagaaggatcctccctacagcctcacaagtaagatgattagtcagcctctacttgaatatttccagtagtggggagctcataacctcacaaagaagcacagcctgtttttggatggctctaatttttagggtgttccctcttatttttgatgaaaatctgcctctctgtaacttccttagtcatgactggagttctgttgtatgagctgaatccttttatttcagaatagggcatcacatatttgaagaccaacattaagtcccagtgtagttttcttttttctgcatgaaacattcgaaggtctttcagtaaatccccttgttttcttggtagggagtcactcatcaccacttttctcttcccactggagctgtattaagctccttgaaactttgcatcctggagatacagctttctagtcacaaagcccaatttaccttctctgagaagagtctagttctttttattagttttagttgtacagtttgggattctcctttttctcctctttgtcttatttttcttcccagcttcctcctcacgtggcgttgttgtgcataaagttctgaatcatatttttcatctttgttcgttgcaattattgtcactctgtcttccttcttggattatttctttcattctccttagaaaatttttgaattctcccatcagctggagaaaaggtatcttttaccttctcctccatcttatactgactatgcaagcagcttatacttaggtcccatgcaagcagtgacttcagaaaaaacaagaataccaacaatgtatcatctcctattgttggctcttggttttgaatagatattccctacccaactagattcatgtgtgcgactctgagtataggtcattcatcgaatgtctgttactgctagagtctttgagtatgtagtctgttgatgtgaacctaaatgtaattggtgttacatgcagtttaaatgtcaccctatttcaacctcatgattctcaatctttatgccaatagctacagtatatttattgtctaccataggatctaattacataagctttggtactgctcgttgtccagaaggatcataatgttagagaggacatgtcttcctcacattatgaaggactaagaaacagaggctcagggagaatatttggctcatccaagattacaccattagttcagcagctggtcagatgctgtttccgataagccactcagctagctaactcccttggcttcaatagagaaagtgacttaaactgtggcatttctggtcttttatactctctctctctctctgtgtgtgtgtatatacattttttcttctttatcacctagcatattttctgatatgtaataagttctcaataaatgctggttaatgaatatttcaagccatcctttccttttggaagttgggaacatgtgtaaaggaagaagactagattgagtgataaagcataaaatataactaggaaagtcatagcttgggatagagagtatatgcagtttgttcagaagtatataaggcattgtgctatgtgccacgatgcctatacagaacgtatagcctagttcctgatatcagtgagtttataatgagataaacgtattatatttggcattgtgaaaatgagataaaaatagagataagactgaatcatatggaaaaaattagtttccagttcaagagaatgttatcaacatcaatgaggagttcagagaagggaaggtaatgaagagcctgctattatgctattgttctcatcagaagaggggtgagtgtgagggaggaagagaggatagatggaagatgagaagagggtgtgtgccagagcactgttgtccatttagtgccagccctgtgtggcaaataggacacaaacctccaattggcttaagttcctgaggtagaagagattaagaatgttaaaaagggaacaaaatttattgagcacctactataggtgaattcatgaatatttcttgatcacctgtctgtggaaggcactgtgttaggtgttagagataaagagagatactatcttcaccttcaagggcaggccaagcagtgtgctaggaacctcacaaaaggaggtgctgaagtcacataagctccccttatgtagttattccagttcctggttttcacgtctaatggatttttccccatagcttctccccctccacccaaccccagcctttccagagtgtgtctgtcttcccaaaatgagtttgatactttgtgcgcatgccgggatccccatgggattgatgacatcatacgccattggctcctattgcacacaagagaggagacagatctgcacattcctgcagttaatgctgagtagaatcttggcagaggtggtggcatccttcatttgactttagaccttcagcctcccttttaaagccctggctcacacagttattgagaagccttatcctgcaaggaacctacagcttaggaaagagttgcccatgtgtctttgctatcaaacaaaagatattggcaaatcttttatcgattttctattctggcagaggtaccatttattatggggtagatgggatatagataaagcatctccctgcccctaatccctgccttgctgagataaatgcctgttccattccccaatcaccagaaaatgccttggagcaatggagtctaacagcctgagagttgtaaaggggcagcaaagccacttctgcaggaagagagtgtgcatgaaccttattcattgtccatgtgtggagttgcttcatgttcatgctcttcaccctgatttgacacataccaacatcaaataactatctactctaccccatttggatactataataaaaaaaagatagtccagttaccttataaacaacagaaatttatttctcattgtcctggaggctaggacgtccaagatcaaggtgctggcagattcagggtctggttagggcccactgcctggtttatggatggcatgttctcactgtgtcctcacatggtagaagcagtgaggcatctctgcggggtctcttttaaaaggaccctgaaagagggtgccaccctcatgacctaatcacttctcaaaggccctacttcctaatactatcacattgagggttagaatttcaatataggaattttgggggaacacacacatccagtctatagcactatctgataaggcttctgaatagtcctgggccattttacataacgggggtttactaaggactctcaacagcatagctcttgcttcaaggggttttccatgcggggcctgccagaatcccctgaagatttgcccttttgcttttgggagaatcattgagagaaaaaggacctggctcctgtgcttttcgcatgctcacccactccggcccttctgattaatgtttgctgttctgctttgcagatgcctcccaggacccagagggagactgtagcctcatttctgtggagacctttggctggactctcctggctctcccagaggtgagagaccgtggttcctgtagcctcaggcttggcaccagctcatctctggtggatccaggattccagatatccaatctcactacagccagggcctctttgtcccagtgtgaccttgtgggctgtccccctgactgcgtcttaacagcccctgccattggtgattttcattccagctacgagccaatggatccctgaaagcgactgtgtgggcttctctcaccatgtcgctctggcctcagcctggcggtgtgctcctctgtggtctggatggggatgatgaactcgggctgcccgttgtttttgttttgtgatatttaaagcagccgaagctctcattactgtaatacagtatgtgcctgggctaaagggggagcagcattccttctggatcagggggcaactctattctgttgtggtacacccgctggagtgtttagaaccacggctggtagatttacagtgccagggctgacaggggccttggaactcctccattttcattcattccagcctcgttgttggaatgaagagggagctgagcccattctgggtatgggacgattgcttaggccctggggtgatgagtggcagatctgggactagaactcaggcccagaggccagcttactgtcatctggctaagtttctgcttctgttctcttcacttgttcaagatccttctgaggccctgaaccaaatgaggaaaattcatgtaaatcttagcacactgcctgatacataataagtgctccataaacgtttatcagtgctctcataagccctgagacatgcaaaatattcttgcccttcagggacttacagtcttgaggaggagataaaaatttcacacatacacactaacacaattaaggagcaatttaagtcactaaatggttatctgctaagttttggggaataggtgcagtggatcacgcaagagaggagacagattattgtggcctagaagaagtgccatggtgaagataagagcataatgtgtatgacatctgtgtatgacatcttttcaactcctggcagtgagatgaacaggtcagcagccattcctggacactgcagagccctcaggccatccactcatctacccagcacattcactgattatctcccaactctgtgctctgcataagaatgtaaggaagaccatgacaagaccctttcctcaagcctagccagggagacagacatgcataaagatactaccactgaaaaattctacattcatatgtgttcatatcctcaaagcatcgtctgagcccacaggtgggtaggcaagtcagataggagcaagagtttgaaggaaagcatacatttacctggtgaaaaggggcaactcacaagccataagctcttgtgcaaaggggctggtaaatgtgagtcttctgcaggagaggtggagttgctgggaggttagacccaagcaagggcattctgagaatgagattgagaagcctctgaggccagcatggggcaagcccttggtgtcacacttggccctggcaatgtggtatttttaacctgggccagaaagtggagaggagcgggcctggtaggatagagaggagggctctttacttatcaagagtaggtaccagatgctggtgactcaaggggaagcttcctgagtggagatggagcctataggtgtttaatcgcctggaacaaacatattttctgcactgcttggttcagaggctgcaagttaaagtaacacagaagagaatgtccggccagcaatgtgaaggaagcatgaaatggaaagggttagaagctgaacttctcttgatcaatgactttattatcttggcaaggattgcagaaactgtaggtgctgagggactgacttgtttggaggaaggggtagggaccaggaaaagaatcccagacacttggtgctaatccaactgctgaatgaggctttcaggctctgggattcaggcaggaactcctgggccatattcttcctaggtatggggctagagagccttcaatcttagtgtctgtatgtccctgggccaatctgctttgttctaccatgcctaacatcagaaaaaaaggggtagattaagtttagagccactgctctgtgtctccatcctgcttctttttcctaaggcaattgagccttacaaggccttctaagtccctatgaggtctacagctgtcagggattggtgaggatggagaacaaaccagtctgttctgaattcaactcttgctttctttctccctctcacaattgaaggtcttcctgagcctttgcctctttcaacagctggatgttgagactgcaatttttcatgattgcttgttttgtattttttcttcatggtctttcctaaagtctgttattcatccaggtccacttatacatcttctaatctattgcaccaaccctgcagccatggcctggcatgaagctgcttgctcattgtcttgttggtttttccaaatgctcacaggcgctagtcatgtaaatctcctcctggcaagaaaaaaagactgactttttgttgttgttgtgttgccccaggtgattcagccagtcctgggctcacaatagatgctaaacatacgattgccacctcgactgctgatctttttcttcctatacaccattctattcataccgctattcaaaaattgatatttataagcacttactatatcccaggaatggtactgtaagttctttatatgcattattccatttaattttaatagtgataccattttctacttttaaccagcttatactttctacactgatagaaaagtgtccctaaacccacagactagttagtacctggctgttttagaagttcctcactcatgacatttctgaagttatcactacaggatatggttgcaaaggcctgggaggagaaatcctttctgtctctgatggtttcagagaaggtgagaggtctctgtctctttgtggtaaaaacaccagggtaggccttgatctcctgagtcttgagtatgttaagggaaagacactaagattgctctgccaaaagggcagacagaccctgcctggtgtctgggaagttgagcccaagtttctctccaaggagggtctcctgcagggatcaggaaggagggtctattggcatcacacatgatctcttctgggttcaggaggggttcctatagctagatgagatagaattgattgatgtctcagtgatgcttcaggccatgttgattgggcctgcactaaaagtaagcagcccttcagagccatgtgctctcaagaggcagcacagccataaccgcaagagtaggatggtcctggtggaaatggcacaatcaaaacaagctcctgggccttgtggctttgagtggctgaactggaaggttcttgaggtatgagcactctcactcacggtattgatcaagcagcgtaatcacatcactgcccccacagaagtggctgccactcagttctcctcagcccaggctgatttctgctctgctggttcataggaaactgcttgggtctttcatgactgctgaagcccaaggcactttccccagaggcagcatcactcacaggatggaccaaatgaaatgtcctggtcaaaggcaggaagtttgtattgttgttcttgcctattttactatcatgtaaatacctgccaaccaacctacacatattctctgagtacttactgagtacttactatataggtgttaatggatacagaaataagagtatggctaggggtggcatgctttttgcccctaacttgcaaatttattgagaacatgatgccaggcacagtgaaaaccaaagagaaatacaaataaatggtaacatttatgagacaggtactaggttctagacacttcttacatgttaactcatttcatccttacaacattcctgtgggcaggtgccattattactcccatttttacaggtgagaagacagaggtgccaaggttcaagtgtgtgcccaggtgcatgacactggtgagaggtgggagacaggactcaaacccagcaagcctggtctcagacaaggtgactcataaggagcagcaagggaggtaggaggcagaacattgagtgcaggggctcagagggtggagggaagtaacttcaatctggtttaaagaaaccagttatcagagacagacagggcctaaagaaggtatgggtggctcagagttttaggaaggaaaaacaggaagttgggctaatagggatagcgtgataagagaacaagtgctaggctggaataccaggggcctggccttgcctgtgagcagctgctgcccctggggaggtcactttgctttttgggctgccttatttttatcacctccaattggagagaattgagaaagatgagctttcaggtcccttcaggtttttaccgtatggttctatgattttgagggctggcatgtttccttggttgcttccaaaagcctctgaccacagtccctcaggctgaccctcttgtgcccagtaatgtggaaagatgaccttctgctcaccagcacatgcttccaagggctgcggagcctgggacagaaggtacggtgggaaagaaaatgagaggagctggttgggaaagatactcacatgcagaaaagtttccagcaagtcagaggtgagagtaaaagatccaaatgaaaagctagagattgctagggctcaccaaggttgaataacttgtccagggtcttagcagactgagggatactggatgataacacttctaaaatactgttttgtatctggaaaatgctctgccttcagtttgcattttctctccttcttcctcccagtaatcaaatgaagacgttagatttgacatttccagtgtgtacagtacagacaggaaactaggtgaatgaagtaatgcagtcaatgttctgtgactccattctgcccctggaagagggatgttttctataatagtcatgtgactcaacatcttcttgtcttcttagaaaggagtgagagaataagatagccatctagcactttcccccctcacctctcctacaaactctagcatctcattttacagaaggaaaggctgagatggaggggagcaaagtagtcttataacaaaggcagaagtggagcttgctatttcatccagtgtgggtcacctcaggtcacctggggcacatcctatgtgcccagatacttacggaaggggtcattctgggcctttggctccacactgaggggctgcattttgagaatcacgaggttgggactcccagaggaaagttgagagttaggaaaacttcaacaaatcctcaggaatagaaacagaacatgagatgagaatgtttgggacagctctgctgtttgtttctgtgtatgtgtttgagggcccatctcttgccaaaaccaagctcatttctgcatatttgtttgcttaatatgaaatactccaaaactgctcttcatatttgtttaatctgaaatgaaaatgactcagcagagaggaaaaacaagatttaaatgccctgaatggcccctgaattaaaatacatagccctaaatcatgggggtgttcttctccccaccaaatcttaatcttcaacacacacaaatgcaagtgccacacacagtcatgcacacacacacacacacacacacaaacattgtatttgcctgccccaacttgacctcagtccctttatctcttttccccttcatactccagcctttccagggtcctacctttcctgcaggggaggtgcatttgagcttaggtggagaggcctggcctgggctacaggccacagatgattttcctggagggccagcacttcctctccagtccagatcacaggctcagtgtcaagatccctgggggaaatctgaattttcattcttcagcaaagtcgaggccacctcagatagtccctagcttttggatctggaaaaatggactccgtggagagaatggctgtctaccaaatcagacatgtcttgctttttacacaagctgtattcctgaaaagttgggtataaactggatcttggtatgttgcatcaattttgaatgtactgaaaagatttgctattcaaaggaacccagcactggattcttttgtcaagggatgaattactttgtaatggctactcatctagtgatttatgagtttggtaagtagatggaatatttttatatactgaatgttattttaaatgtactgtgtgggattttttattcaaagttacccggcattgaatccttttgcgaagaactattctgtagtgtagataaacatttcatgatttatgagtttggtaactttaggcatttcttaaagtgggatatacccctagttctgcttcagagaaaaagttgttttttgcaatgatcagacttggagaaaaaagagttctaccatatttgagttatggtcactcctgaggggcttaggaagaatggaggtactcaaactttccattttgttggagcaaaagatccaggagttgcctgctcttcttactttccatattatcactggggtggaaatgatggaggtatttttccaaagaaatgtagttcactattatagctacatttcaaaggtgtttctcttcatatctatatcatttgaatatatatttatatatatatacacacacatacatatatacacacacatgcacatatgtttatacacacacatgcacacatgtacgtatatacatatataatctatcatgtgtatttagatctaaagcaaagacattgcagtagggcttgtgttttgaaccactgaagggctgctgtttccaccagtccagagtgctggggcttgctctgagatcaccaggggtataaaccaggcaagaggagaacttaacttcctgctcactctttctcacttggcaggtcaaaaatcatttcagcacttatctcccaaggttcctagctgtgtccccataccatcctatctgagcactagtgtgaagcttgttttgggaaatgatctgattgctaaggcagttggaaaagagaaatatggtgtcatgtgtgtacaaccaagagctgatctaggatgcccacatattcctggctcatccatgagccacatattcattgaagttcttataggtaggagcaaaccctgcctataagaaccctacatggactggaactgtgtcaggcagaatgttcagataacttaccctttcaccatgatttggccctgaatgtgtctcagaaaaaggcttttagaagctcacccaagtacagagataatttgtgagacatttaacctctcagactcttgcttttttctttcctggagagctgctaatttctagttgtggctgccacactcctgtctgcctgatcttccattttacttcctggtttcctggtggctttgacacatggcctgtccagatcccagtgggggcagtcctgagatggagagctggtggtcttttgtcagatctgggaagtcctgagccatgacttcctttgaaatcaatttggaatgtccatccagcttggctagggaagaggattttgactgtgaagagggaagtgggttctttactacaaaggacatattctctctgagccacatgctccttgatgttctttggtaaatattgggctgtggtcctagaaacactgaccttggagtcaggcgaactgggatccagttcttactctgtggtcttctaacttagtgacccaagcaagtcaaaattaatctcatttcgagatagagatatgcaagatccttgaccttcaatgactcatggtcaggcttgagagaaagtcaggtaaagaagccaactctaattcagcacaatgaatgggaagcaccagccagaccaggtaaaacagcctcaattgagggtgaagtgacttagggccgtacttttctattgtcaaaaggagatgataatttctctccacaggattgtggtgaggctcaatgatgtaatgcatctataagtaactgataaagttcaggaattctaaaatagaaacaggcagtaattctcaactgttttcttcttccactctattcatgtgcaagatgtgctaagacatttcccaatacaacaagtgattcttgaagtataggacgtgttccccccacacctccaactctcggaaatgcacagaaagtgtccttgagattctgatagcctgcttccaggaaaaatgttttgtttggttttgtcagaaaagctaatataaatgacattaacaaggaacacattttccttgttgaaaatcaaaacattacaaataaggctaaagtctccctttgatcaccattcacaatctcctctgacaaatacagcagtccatctcctaactctgtcttggagggaaccactgctgtcagcatggtgtttaatcttccagatcttatatatatataaatgtacatgagtgtgtgtctatcataaaaatatatagtgtgctttacataaactttgcaaccttcttgttcacctaaaaacatgtcatagacttcaaccgttgtaactgtgagtagctctaatgcattcattgttactattgcatagcatttcacagtaggaatattctatagttaataacattatcctactggtagacatttttctaagtttttgttgttaaaaaatatattgcaaagcacttttttgtgcatgcctcattgtgtacacacacaagtctttctttagggtaaataagaataaatgggatatagaaattatggtatatgtatttttattgaaattatcttctaatttatatatccagcagcagagtatgagagtatgcatttgccatattattgccaaagtttatattatcacactttaaaattttgccagtctgataggtaaaattggcattcaactactgctttaatgtttgtctttgtgactacttgagaggttgaatacttttagatgtttacagcattatattgtctcccctgttgaatgctttttatttacttggcccatttttctagtggcttttaaaaaatgatttatagagattattaatactagaagttatttcattatacaatttcaaatatttatcttagccagttgtttacatgttatgtttatgatgtcttttggtcttaaagaagttttgaattttggtggaaatttttattattcttttcatttctggattttgctttccatgtctcatttgtgcaggccttccttatctttagctcatagcaaatttttctagattttccttgaaatattcttatggttccatctttacattttgggctcatttttgtgaggtgtagatataccttcatttgcttttgaaaaggctactaaagcatctgaaacttcttcaccaagtttaaatacccaccttctcatgtagtacatttccatttattcatgagtcttttctgggttgttcattctcttcagtgttctgcttatctatttctataccaattgcatactcagagaggcctttgctggccaccttacctagaacagctctcattccctttctgttacattactctattttattttcttccctgtacatatgtttctttaaaattcttcttcatttacttgttttcttatgtactataagttccatgttggcaggaaccttctttgccttttttactgatgtgcttatcacctgtaatagcacctaatagatatttacaaaatagttgctgaatgaatgaatgaaggccaatggcttgaccaggggtgtttgcattttgagttttatttcttgaggcttaaataaattgtcaaaggggtttgtactactaactaaataagtgggtagagaagggctttcctagcagctccttgacatgcttccatgtatacttttgagttctgttcacatgtcacattgtttaaatattgaacccttggaaagactcattcgaaggtgcagaaaagggtgattctgctcacaataagctttagagatacttccttttagggtatggaatttagaatgaccagatgtagtcactgaatatatggctcaaggcaatcattaattcattctgcaaatacttatcaggcactctggtagtgttgagactacaacaacgaataaaatgaacatggagcttgtgtgtgttgggggggaatacaaattaaacaatcacagcaatgaatatataatgagaaactatgatcatttctatatagaaacattatgtttttagagcatgtaacaatcaaggggatgagagaatattttgctgagaaagtgaccatttagtagagatttgaactaggcaaagagaggagagaaacttcttggcggaggtccttaggcaaaagaaaatggaagaaaaagaaaagaagctccagtgtgactgaggtggagaggatgaggagaagggcagctctggtgaggtaggtggagcccagatcaaaggatgatatagtgtgatgatgagcaaaaacaaaagaaggaacaacaaaaacaacaaaaacccatgtggattttgaagtaacacaagccttaggttcacatatgagtttggtcacctatcaggtgtgtaacctgattagctttaacctctttctccttagcttccttgttgttgaaaacaaacctcattgagttactgtgaatattgtaagagatggtctctgattcgtagtagaagtacatggtataggtataatacttctaggtgctttacagtgctctgtttgttagctgacagtcatctaattttaggcaaaacagaaaaataattattgcttttggagattcgcagcttgaagcccggcaacagtttcaaaaagtaaattagtcaacattttttgaacacttagtaggtttggcttagttcagttaaaaaccatttttgagcacatactttgtgcacgatgtaaagtaatactttggcaatataagaatgaaccattttgaagttaatgattaaatggataattttataacaatgttgtgaaggatccagtagagttaaattaagcatgtgttttggtgaaggaaagaaaggaaatgatgtgtgggagacattctttgcagaaggaattattggggaaggttatggaggggaggaagttacggccagtaacccatgagagaggtgccacaggagagagagagaattctaggactgtcctgcaaaagactgctaggagagggcggtgagccaagtcagtcctgggcctcagcacttactggatggctgcttacaaagagctcgggtctctctggaaggacatcctggattatggtcatgagttagtgctcaactatagccaagaccaatgtctccaggattcacctgtgtgtttctttctctgggcatgtttatttagggacttatttatggaaatagtgttcccgttgctggaagtggctaggaacactgagacatgtattttatcttatccaaatcaattgctatgaaactatgctcatcaccctttgtagttgtagatcacatacccctcagcccctcactatggggccatttaaatcttccgtgttttatttgctttggatccatcctcttcagtcaaaaccatctgactacattgcttttttttttttttaacccctctgggtgtgtcagtattatactgcttagaaattgtcttagaaagcttctgtttcctgaaaaagaaaactccttactcatgtcatcctctgatttactccatccctgttattgcctattaaagtcttgtccatccttcaagaacctgatcagatatttatttctccatgaagccttccttaatcactccagcttaagggaatttcttctctctttataatattgtgtcacatactatttctaatattgcatccaatgcttgcttgattattagtgttactcagagggaattttttaaaaatcactttgtattctagaacagtttttttttttttgtctcactctgtcgcccaggctggagtgcagtggcacaatctcggctcactgcaacctccacctcctgggttcaagtgattctcatgccttagcctcccaagtagctgggattacaggcacccaccaccaagcctcgctaatttttgtatttctaacagagacggggtttcaccatgatggccaggctggtcttgaactcctgacctcaggtgatccacccgcctcggccttccaaagttctgggattacaggcgtgagccactgtgcctggctttctagaacagttttatatttacataatcattgtgaagctagtacagggaatccttgtatatcccacatccagtttcccctattattatcatcttatattaacatagtacatttgtcaaaatgtatgaactgatatagatacattattattagctaaagtccattctttattctgatttcctcagtttcccctaatgtcctttgtctggatataacattacacttagttatcatatctccttaggcaccctgagctgcgatagtttctcagactttcttgtttttaatgaccttgacagttttgaggattactcatcaggaactttatagaatttcccccaactgagacttttctggtgatttttttataagtagactgaggtaataggtttttgggaggaagtccacagaggtaaagtgtcattttcatcacatcatatcaagaacacatactatcaatatgacttatcaatgttgatgttaaccttgattacctgtcttgaggaagtgtttgtcagatttctccactgaaaagtgaccctttttctttctctgtttttacactgtacttcttgggacaaagtaactatgtgcaacttactcttaaggagctggggaagggagatagtaatgcttcattttcttaagagcagattatctacataaattatttggaattcttttgcattggaaatgtgcatattctccccatttgtttattcaatcattaatttatagcagcatgaactcatggatatttattttattctttggtttataattcaatactgctgtttttgaggggaggaactcattaagttggctactgtgtccctttgtcataccccatcatctttttgtgtgtacattttctcaatttctggcactaaaagatactccaagtccatcttgtttatagtctgctccattcccagaatcagccatttttctgaggaatcctgtctccttttattgaagaatgttattagaaatcaagatctgagcactaggtttgctcattgctactgggatgtcattgcctctaggccttttcagcggatggaacatatgtgtgtatactaacccaggcatatacacatatctataaatatttccatatgtaactatttgtatccttattaagctaaacataagttcatactgatgtctccaaatctaatctattatcatagattagatattatgaatcattttggggaaacctgaaaatacagaatcccaggtatcactccaaagataatgaatcgggataaatattgaacttatatgttttgaaaggcaaacattaaaagtaaatgcttctcttggttgaatattggaatcaatatcaagattatgaaaacatcagtttacatgataaatataagttttactttaccattattcaccatttgggtaagtatttatcttaggaaaacttcgtgtgtctgactatacttttgctaatttaaatttcatgagtgtgcaatctgatagagtttgaaaattattatattaggtgattattggatgtgccattcaactcccaaagtctgtgattcgaagtcccatatataaaaacaaacactgcaggaacacaaaattaattgtgagaattgtactcattcatgttggcagtatctgcctcactcatttcctctccttctctttctgttttcttgcattattggttggtggcagactccagttccaacaccatcttctgagatgatcctgattcccagaatgctcttggtgctgttcctgctgctgcctatcttgagttctgcaaaagctcaggttaatccaggtaacatggctattactcagctatatgctagaagaccagcagaatgtttaaacccatcaatgttcaatggtgggtctaaaagctgccaagagggactacaaagctctttctttttaaatctccatggtgaaatatgactaaaataggaagaaaaatgaatatactttatatatatgtattttaatacttacaacctgttgaatattgtaaaatattttcttatatagagtaggggtgcagtaagagggcattttaaaaaatgggaggtggcagaagcaaccatttgcttctgcatcttctgagaaaaacaggacagaatttttagatgtcttggagaacttcttcaggttggtttaccagttgttttgggctgtcttagactttgattttacttggatgtcctgttatgcttttgggatcctggtcctgtgggatctcaggcaggttctctttcctctaggatgtggccatgctaaaaatagataggttaggagaatcttttgagcccaaatttgtgggattattttgtattacatgcatttttaaagtttaaatggaagtaaataattgtattatttattacacaataatgctgcttaacaacctatccaaagctcaatgtctttaaacgagcatttatttggctcctagctggacttgtttggactgttctctgttctgtgttgatttactcaagcataactggaagtcagctggctattggctcatctaggatggatgtggcactgatgaatggggcaactttgctgtgttctgtttcatcctcctgcagactaacctgggtatgttttcctggaaattgcacaggaacaagaccagaagccagaatactcaaacatttttcaagccactgctgaaatcatgtttactaagtctcattggccaaagcaattcatgtggccaagcccagagtcagagtgggagagcacaaaaaattagatggtaaaggtctgggatgcaggtattggagaagaattggagtcattttttttttcattttgctacaaatatgaacatggaaaagtatacagcttaaatgttcaattttatgaattcataaactgaacacatctaaacagtacctggattaagaaacagaactataccagcactttagaagctctcttcatgctcccttccagtaacttttctcaccaccccgcaaaggtagccattatcctgctttctagcaacatagattagttttgcccatttttgtactttatataaatggaattagatgatgcataaatactctttgtgtctggcttctttcactcaacattttgtttatgaaattcatcctactgttgcatgcagttgtcaactattcattttcattgttgtatattactttgtttgaacattttatttagccaatctactgtagatgggaatttgaataatttctagtttgggcccattatgaacaaaactcctataattattcttatataaaacttttggtagagataagtactcagttcctttgagtatacacttagaagtggaacttcatatgtgtagcatatgatatactattctgcataataccaaaaattttttcaagtactttaccattttaccatcccatagcagtatatgcaagttccagttgttccatgctatattttgattcttgagggtgctttaattttgtcctcttcatagcataagggtacatttgtgaaaatgcttgctctcctcccttcataaaagttccatgtgcatgggtttttctttataccttcaataggaaatatgatcaaacatccttaaaatcaaagaattattggctgaaggagcccagtgccttgttactttctcgtaataagctcagctgttgcatctcagatcctaacttgggtaccattttttcctttttctgttctgtattcctctcaatcaattgttgatgtttggttgcctcctactacattcttcatgctctgagttctaaggaagagtgctgtgagtgcatggaatggtgggatgttttggtggtaagcatagtcatggagactggcatgccacgaggacatttaaatgactaaccaggagagctgatcttctgtgttagagtccacactgtggccctggagcaggaaatgggctatttggagacagcaaatagtcatcttaatttttttttctatttctggtgtcaggtatttttgttggctctttctgacagggaaagagaaatagacttcttgctttgcaaagacctttataggaaaacaggacatgttgaaggactaatccttctctctgccatttctttctattttaccaagtcaaaccagagatgagtgtttgaggcagatgagacattttcattcattatcagtgataaagagtctatcatgaatcctgaggctcatagaatgttatagcttgcacactgctgagcataaagtcagcatatgataaatgcatgttgatttaaggatgactcttcaggagtccttactggtgtcaactgaaagactgactataactgaaagtattgaaaggtaactgaaggcactgaaagactgagtgaattcttggccagttaggtttttcccacagaatggccacatgatataacaaatagaacttaagccaggagacctgagttataagcaaatccaatatgcaaccaattattaaattactgaaacattttagttggttattagctataacatattggttggttaatagtaactgagctaagcattaaactcaataaaataaaaataaaataataattagttgaagctcattgttgaagatcttacaatgactattggtcaataagccttcagtattagcatgatttagttgataacaaacattaaacatgatatgatatgaaatatcaatcttggtgtctcagcagataccaagagaggataaataatcttgtttacatttaaaaaaactctctagtatttgaaatcataaatgtctcacaggtgtcatccatggaggctgcagagtagtggtggtatgacagagatagttggctgaagcacgaaggcagcagagtttttggctcactgttctaaagtatctgagttttggcaaagcaggcagtcaggcagatggtgttagtggcatttccagtaatggcagtgttaaaggtagcattgtcaatggggcctggagcgtaggctacgcaagtctttgtgggaaatgctgacatgaaggtgtatatacatagtcctgaattgtgggcagagttctcagctatcttccagctgcaacagtgcaccaggaactggaacctaatcatcattaatccagtgggagaggttattggaagaattagaggcagtttcttatgtcaaaatcatggctcagaaataccatttagctagtgacatccaaatcaaacaaataatcatcccaataatttctgaaaatcatttatacaaatcaacagatgattggaatgttattagttctgatgaatgatttggagttaacttagaaaaaagtatttagcttaacttttgagtaacaaccctaattataatcctgacttggtcataaattaattgtgtgacagtgagggagttgttacgtgtctctttatacttccttaatattttcacttgtagtaagattaagttggattagataatctatgggaccctttctgctacaaaattactgtgatttttatgatgtgagaactttatagcccatactggaagatttcggcttggaagtatagcccctgaagttgcatggctgttttttgcaagaaagactacttgtttccaactccctaccacttcccaggctcagggatgtgaagtctgaatttcaggagaatcttgcctttggggctaagcacacaggaagtgactgataaatacttaaaaattttaaaccctaaaactctgcttagtggcaggtgaatgcagaaaccctgccccagcctaacctgcaggagagtcatagtgctacaagctaccctgctccagagcatcttttgctctagaggtggggccaagaggtagcatttcaggctgaagatatagcgtatgcaatggctgagaggagggtgaggaacatttggggctcctggtggaggataatgagtgagagagccagcggggctatgtaggagatccattcccctgtgggatccttgagtcaagttttcccttatttccagcttgggctgggactgccaaacttgataggagaactaagggcatctccatgggaaagagaccccacaatcatgcacaaaaatacggggctgcagtagttgtttgagtggacatcagtgtcaggctcccctaggggatataaaaccactgtttcagtggttagtggggaactggcaccttacgtttttccaggtaggaggatgaatagaactatcctgatgatcctgaaattctagcccagcagtctgaagccctgggacccacccagccaattagaggattgttgtgtataaaaaacacaggggtgcttgttcttattcccctgacttggcctgtgacaactgtttgtgctcaggaaagggagaaacatctcttcttctcagcaggcaaaccaggcctttaggatccatggtataaagctgaggcagggcagtgctggaacttcccattttcatgtcacactttcccctcataggaagcaggcaacttactaccagttcagagtcctggggtgggagtccccctttggtatccagaatgcttccactttgaaaaagagtggcaaagccattcagggtttgggattaggagaggacgagcttttctcctcctggccccaaatttattttaggttcaccactgtggtttttgttctttccagcatcaatcccacatgtcagtgagttcttagggtcaggcaactagaattctagactgttcatgttggaatggaccttagaaatatctaagtcactcttcctcactttacagatgaggaaactgggagtcaaagcaggtgagtgatttgcccatgctccacatttagctaatagttaagctaggacgaggacccagggctacctgtctcccaatcccaggtgacttctacctgggccctcagggagcaatgtgctgaagtgacctttatctaaaaatcacatatatccatgcagcctcatgctttcctttctgcaaccaacttccaatgtatatttatggggatgagatgtaattgagatttgagggacagcctgaggccaaagaatggagtcagacgaataggatatacagtgctcacacctagtctcatgctaaatgaggaaggctggagctagaagccctgcgggccaacttacccccacccagaaactgaagcaccagggatgctttggttcccttctgcaccaggttgtgcctttatcaccgctcccttgtcttccaccaagactgctatgtgttaattcacctacacatatttccaaattggaaactcatttttgtttccctggcttctcgtcattcgtgttcccagagatgtgctgagaaggtttatgtggcctcatctgttaacgagaaggtgatgctagtttcaggtttgtgaaaccaggcctgaggtactagtgttgtgatgtgtgtgtatgtgtgtgtgtaggtgggtggcgcgggggtgtagtgtgtgttagacagcctgaacaggccttgagaaaaagataaaaggccggcaaaagacctctctggttgaatttatcttaagtgagcaatgctagaagctgcccagattggctctttcttgcttgtgtgtttgttttttaagtctaggctgacgcctgagagtggcctttggcatccttcctgccagtaaaagagcctttctctgggtaaaacagaaactcatctgccccttgagccctcttggttgtgccaaatagggaaaaagagaatgcaaaactcaaagaatattcaatgaacacttaatatattctgggtaccattttaagtggtgtcaacaaatttaatcttcacaataatcttattagtattagtgttattatataatagtagtattattattccagttttacagaggagaaagtgaggccaagaaacattaggtaactcatagcaggttacagagaaagctaagtggcagaaccggcattcgactctgctccagcaagcatgctcttttcttctacttcttttttctttatcccgtttttcaaatttccaacaatgtatcagttaagagtttttcgcttgtttttttgtttgtttttggagagggcatgttgctctgtcacccaggctggagtgcagtggcatgaacatgactcactgcagcctcaacctcctgggctcaagccatcctcctgcctcagctccccaagtagctgggactacaggcatgcgccaccacacccaactagtttgggtactttttgtagagaaggggttttgccatgttggccagactggtcttgaactcctgacctcgccctcctaaagtgctgggattacaggcatgagccaccacacccggccacaagcatgctcttgacctctgtgctctactgcctttggagtgtgagactcaaggaatgtcagggcagaggaaacccagattcatggaccagccacgaatgggctacagagacccgtgaatcccctgaaattgcatcccaaacttaatgtgtgaagacaaaagtacatttgcaaaatgaaaaatgttttctattatcagatgctcaaaagactctgtgaccagttctagaaaagactaagaatggtggatttaatctaatcttgtacaaatgaggaaactgaggtttagggagataaagtgatgggaggaaacatgtttgaaaagactattagcagacctaaccaggctttgcctactcgctggacgagcatggtcagagttttgggttctctgaactgcagtcttctcacctatcactgatcgctgccttaagggctgttaggaagagtaagagaatatatgtaaagtacctagcatagtgtcaaacacacagaatgtgattaataaatgctggaacttctgtcctcttcctcaattttgtatattgtgctgtgataatggcagacttcattagtctttccattattctatactgtcctttatattttagtttaatcatttcattaattaattatttaacttaattactatactgtaaaaatatttcattttctttgagggcagacttcacctaattcctgggctttgtatcttgggataaaagggttccaaatacatctactgcatcatcacctttccatccaaagtagccaggtgaattcccggttcataccctaaaactttgccttcaccctgttgttttgcattctgaggcctagattcctagttgtaaaaggcagtcagaggagctaaaagtaatgcaagaaaaaacaaagctccactttttttctgttgtcagtagggatagaggcccagggagacctagtctccttgagtctaaactttggggccaggagcatgatcatgggactccaggaagagcttcacacaaagtcccttttctggacctctcagttttcacaaccacctgcagagctcagttttgctcagggctctcgtgtggaaagactaagagtctacagaagaacaacagtgttggcaggagcagacaaggagacagggaccctgcttaccatggaaactgggcgcactggggccattaggtccgtgcatcactgctgcttcacatatttgcttattaatgttttgttctgaaagctgacaagaactctaggggcttcaagcccataagatcaagacatacaatatagcgatataagtatgtaagaaacatagattgatacagatataaaataggaaaaggatatgaaacaaatataatatatatgataaaaattataaatattataataaatatattataaatattataatatttaatattattaatacattaaaatataaacatatatttatattttgtaaatataaacatatatttatattttgtaaatataaacatatatttatattttgtaaatataaacatatatttatattttgtaaatataaacatatatttatattttgtaaatataaacatatatttatattttgtaaatataaatataaatataaatatatatataaaatatgtataaaaataaacataataaatattataataaatatacacacatatatatgtgagtatatttatgtatgtatttatatatagatatatctatatttatgtgtgtatatttatgtaagtatatttatgtatgtatttatgtatatatagatatatctatatgttcacatgttacatgttatatagatatatctatctatatatagagttaaaaataaataaataaataaataaaaataaaaataaaaataaaaataaataaataaatacagagaagcctgggagtttccttgacttcctttttccccctccctcttcttattcatcaaacatttgttgattgcctatataagccaagcactgtaataggtgctggtcatgacgacacaacagattatcactacaccagagacaaagaccaaagtcataaaaacctctgaagcaggaagaaaaaaaaagttgcataaatttttttaacaactcatcacctcccacttaggctcataaaaaaacctcactgattttagtgtcttaacttctactttactgtgtatgtgtccaaactactgaaaaattctgtgagggctatcttgccacatccatactcttcagagacccacaggccaggaccaagagctaagtcgacaatcacactatatatatctatatatatatatatagatataatcacactatatatatctatatatatatagatataatcacactatatatatctatatatatctatatatagatataatcacactatatatatctatatatagatataatcacactatatatatctatatatagatataatcacactatatatatctatatatagatatatctatataaatatatagatagatatctctttatatatatatagatagatatatctttatatatatatagatagatatatctttatatatatacatacatagtccataaatatgtacataaatatgttcacattttacccaatcaggtcaaatctccaatgtcatgctagttgagatactgaaggtatgagagttagctggagctgaaagaatttgccaggccaggtaaaaagaggaagggaaagcttttgagctgaagaaaaggcacagaggcacaaatagaaaaacaagagtgaggatagtgattattgaggggtcatgaaggcaaaggtggggttaggatggcaccaaaagtaggaaccatataatcttgggatcctcagtatatgacaaatgttttatgatcaaaggccttgtcttcacctggtgttaacataatgctatgccatgctgtctttcccagagtgcaggttccttttgctatcacatttcaacaaatcctgggacaagcacattctaaaaattagacaagcaataatcctccctgggctctctgtccagattcttccccaacttggtgattcttgaccctagtgatcacatgataaacagggtgtgtgtgctggtagcagggtgtgatgaccacgtgatacggttactagacagactgtgacaatggagatcaagagatctagattctagttccagctctgtcactatctagctttgtgactttaggtaaatctctgcacctcactttgctcatttataacatggggtccataaactttgctctctgaacttcatagggttactgcaggggattgaaagagataatggaaataaatgggctttggaaaattacatgcaaatatcaaagactgtgatttgttacttttggaagaaaagaaaatgaaaataaacactatgaaacaacagtttctatcaagcagatgatgcaggaggaagaaagagaaaaaaactccattagtgatcattaaggtcccttgtggtccccaagaccgactcaagttgcttttggggaatggggcttctaacagtcctgaactctggccaccctaactctaaagggatgcaggaccctgtgctcaagccaagcatatccatttatatatatatatatatatattttttttttttttttttttccttgggagaatagtgtgattgttgacttagctcttggtcctggcctgtgggtctctgaagagtatggatgtggcaagataaccctcacagaatttttcagtggtttggacacatacacagtaaagtagaagttaagacactaaaatcagtgaggtttttttatgagcctaagtgggaggtgatgagttgttaaaaaaatttatgcaactttttttttcttcctgcttcagaggtttttatgactttggtctttgtctctggtgtagtgataatctgttgtgtcgtcatgaccagcacctattacagtgcttggcttatataggcaatcaacaaatgtttgatgaataagaagagggagggggaaaaaggaagtcaaggaaactcccaggcttttctgtatttatttatttatttttatttatatttatttatttatttttaaatggagtttcactcttattacccaggctggagtgcaatggcgcaatctcggctcactgcaacctccgcctcccaggttctagagattttcctgcctcagcctctcgagtagctgggattacaggcatgcaccaccacgcctggctaattttgtatttttagtagggacgaggtttctccatgttggtcaggctggtctcgaactcccgacctcaggtgatccacccaccttggcctcccaaagtgctgggattacggcgtaagccaccatgcctggcttttttttttttttttgcaaaaaaaaaaaaaaaaatctaaatgatgccatgtgagacttgatctcagaaagatgttgggaagctgggaaggaacccataggaatatcatccctttaaatgatgagatgctgagtatggtgtgagtaaattagaaggaagataatgaaccgaggccatttcagctgaagctaccaaagtgcagcaccaggtccagccttctagtgttagagaactgggagagcccatctccttgcagaggtagagcagcccgggactgcaggggagggtgaggccttcatgcttgttcctaaatgcctaaggtcttcgtttaggctacaagcggagcattttgattgttatacataaaggaaaatgtggttaaatttaattcaaatatatgtaaatatcttatgattggcatttcaagggaggaagtcagattcaatcttttggctactgtaacctatattaaagataggaagtatgcaatgatgtgtactgtctatgtacaggtattgctaacttgagaaatgtattggcatgggaggagccctgggcttgcccataggacaaatccacctcgaaagcagagcagagtgtcttcattctgggtagctggtaggatgtaagcctgaattcttggtcttccagtgcttcttaaaaactcccacgtgctctctttcctcaccaggtcctgaactctaatctgtgtcctgtccgactctcaaaaaatcagctcagctcttcagagggcttctccttacattccccctccacacagccccagcatttggcacatgtgctgagtgagatacactgcatgctcgagggataccaggtctccctcaaaaactctgctaattttgtcccgtagagaccgtctgctagtgccaaacccggagtcggcctcctgcccctgctccaaatgggcaaatggctgaaagtggctacagaagttccctcacctctctgagattctcccttctctgaagtcttggtctctgcattctcattgcctcagaaacaatcagattccttcaagtagatgatttatgaattttatttagctcttccagttacttttagagggagcactaattactgcacactactccatcccacctagaaacagagatattaataaaaagtgaatttatactttattgccactgtgattcatgttaactaatcatgttttactttttgacttgacttctaggaagctcagagccaaagttgaaacccatcatcagccagtggtatagaaccaaatgtaatactctttctgtattaaccttagctctagcttatctaattgttcctattttctttttcctttatccaaatttcttcacttatttatttatttatttttatcttatgcaactttggaagccgactccaaactttactgagtgagtcaaagtaaaaataattaaaaataaataatacctaactttattgaatattagtgctctaagtgcttgacataattaactcatatattccttatggcattcctataaacaagtgcaattactaccatgaaattgagaggctgaaagcttaagtaacctttctaaggtcataaaaccttttaattgtttaaggaagagtgaatgtactattttaaacaaatagttctctcttctcctcttagatatcatttattgagccccattacattctataaagtatgctaacttctttgtatacattatgttaaccttggcttggcaggattatgaaagatgaagaaaccgaagttcatagaggtttggtaactggtagtggggtcccataattcagaagtggcagagccaagtcttaatataaactttccatgattccaaaccgtggcctcagttttcttcttcactcctgatctgcatagctacccagtttcctcttcagcttaccttcttgttggggaaaggagatactatttactatgaaagtttcactgtctttgttaaccaggaagaagaattcattatctgatccctatgttagtatcctgaaacccactacagcttggggactgctctcctggggctctgcaagaattgtttctggaaagtattttgggcttccaggaaaactgcaatcctaaaggtctattcctaaggggaatttcaacttgtgcaagaggtctgggtgaccattatacccaaagaatagccctgtaggctgaagaaagaaacccaaataatctttcttggcccccctgtggaatcccattgtgtcaatatttatctgcacaaaggaccagtctgaaacagcctgctctgtgaagcttgtgggaagcagggcctcagtgacagtttctttaagtggactaccttgtcctttcagaaactggaagaactagaccatggtagcctagggttggaggaggaagaaccagggatcaggagacctgaatttcagtcctggttaccctagccctctgtgtgaacttgagtaagtcacttcacctttctgggcctcagaggttctgatatttaacacggagacattagagggattggcttcttaaatttgcttctgggctccaacgttttatgattgcagatgtccctttagattgtagactggcttgagaccgctgaccccaataatgcccctcagtcagtcttgtctgggcccagagaggcccaggtgtcaggtctctgcacctcaggcctcatgtgggccatgtgtgtgttttcctggaagctgcatataggacactcattgacggggagcccgtggtctggagctagtagtgagatggtaaacattatggaacaagcagccctttcaggctctcagccttggcaggatagtatgaaaaataattcttgggaagctggggactggtggcagctgcttctcctcagcctgcctgccaggccctaccagagcttggagggctggagctccctgctgggcctgccacagcctgtgctggtagttttccactctgggaaactggaaaagtgaattggcttctctgtccgggagattggtgggtagcggaagcatcagaaaaccgctaagacaactcagcaggaaaaaccaggagacacttctttgctgcagggtctcttgcttcccttgccattcccagctttttcatgcttctttacagttcatgatagaacaaagacagactggtgggatttgatgaacgaggaagtggaactgggggaaagcaaggagacctgacatttagggcttgggccaacttggcagttctgagtcttgactattggctgagatcccttccacagaaggagtttacccaaaataggcacattcaatgggaaaaccacactggtacagctcagaagtgccacgttggttgccctgggcttttcctggttccaaagattcagtaggatgagaaaggtcatccaagatcatcttgtccattttcctgtgccttttctctaaggcccttgggcatactgagtcactgagaaactttgcctattttcttaatgtgactattgctgatttactaagttcttatctctgaattgtctatcctcatccttatattactttgtcacaatttctagacctccatcctaactttcgttgtctggagattgtgtgcaatacttgtagccctctctgccttttcatctacttatttatttgccaaacgtgtgttaagcatctcatgggtgctgtgtgttggcaggtactgagggggaaaaggaagaatctgacatgattactattctcaaagttgctcacaatttcaacaaggaggtaaccaagcacaagagtaacagaaaatctccgttggagtttgttacttaatagagctcgcctacacaccatcatcactttaatcccataccacgttttagtattttccatagcttcgtcacattatctgaatagaaagagccagtaatagctactgtcaaatacataatcaaattgtgcctatttgattatgaaaaatctattttcttttttctgtctcccctaccggaatgtaagatccatgagacaaattattttatctgtcttgcttactactgtatcttcagaagaacacccgatacacagcaaaaacttatttattaaataaatacatacacatttgtaaaagaaatacatttgtgaactcaatacatgcaggtaaagataggttgcaataaaatatgtcatgactcagacttacaaggaagtttcctgtatctcctgtctgctaaaacaccattagccaaggctacccttgaccaaactcccaggactttgtttttaagccttgtaaagtgatgaacaacagaaaacacttcctttatcacttttcaatagacgtttattgagcacttggttaatgccagacatcctactagaaattggagatacaacagaggaagatagatatgatccttgccctcataacacatgctgtttagtgggcagaccaaaattaaacaattatttacattacaagtctaaaaactgttatgaacgaaaaccagtttgttataagagcatataatagaaattcctaacttagctggggacttgggggaattccttgggggagtgactggctgacagctgaaggatgagcaggagctagtgagtgaaggagggaggatgcacatcccaggtaaagggagcatcatgcacagagaccttgaggctggaagatactttgtgcattcaggaactgaaataaggtcagcgtgcttggtagagagcaaagaacagggaatagccagagatgaggccagagagtaggctgagacccaatcgtgtggacctcaagggtcacattttcttaagaagaatgaaaacctgttagaaggttcatgacagaataaagttttaggaagctaatttgggctattgtgtggagaagggatgatagaggaacaacgaagaagagctagaaggcattatagtggctcagatgggaggtcatggaggttgcgcactgcatgggaagaatagaggagagaaacagctggactcaactgcaggaggtaggatttgtagaacttgctaatggactggatggttctcaaaggccaagcttacttctcattccttgactgccgcctgttgagagtaacatcttacctatcattcctggactcttcctctccaagaattctattttggtctccaggcagaggttgactcagggagagcctttccagttttcatcaatgtcattgccactctgctgtccaccacacagttgacctaaaccctctgtatgctggaagagctggacgagtcgaggaagaggggagcgctgacatgtgtaacaagttatttacatgaaatgaagcctcatgtcctcccctacttcctctagttctcaagcttgagtctgcattagggtcatctagagtagtggttaatacactgtttgctaccccctcccagcaaccccaccagagtttctgattcattaggtcaaatggagactgataattagcgttgctaatgagtttccaggtgatgctgatgctgctggtcctggaagcatattttgaggatcacagctctaaatggtaactgaaatgcaatcacaatacatagatcaaagagctttcaggaaggaaactggcactcctttgacaccacatagcagagtatcctatatggatctacctccacatctgttatataggaaaattcctgaatgggttcagttaggacacaaaatataaagggtttctatgcctaggaaatataatttaactgaaatgtgtcataaaagcagctgcagcagcagtagctctgataaaactccttagttgccattctcagaatgagcagtacgttagaaggaaagttatttgcaacaatatcatgattcacctcatgtgacccacattccctatttctgctttactttagatattccaagaagcagaggctctcattgcattcatggcctagtgtttagagatgcattaaagccagggatgaaagcccagtgggacggtcagtgtggctttctctggtgcccagcatggtcctagagaatcattacacgtccaggctgaagggacctgggagcagtggctccagagcctggctacacagcacagtcacctggtggactgtaaaaatatgcaggttactgggctcagcttcaggtataccaaagcaaaatctctaacaggaggctcacaaacatgtattctaaaacctctccaggcttttctaatgctattggtccatggactggctttgggaatcactgccttacagaaagcctaatagaactctcttgctttccacatgaagaaacagaccgaaagagagccaggagcttgctcagatcccacatggtataagtggtagaaccgggagtggatgcgaagtcttgattttcaagggttgtgtgctcagtggaattacaggctttagagccaagttctaagatgagttagtatttactcaaggaatggcctttgcagttaaaacttacctgtagaatgtgtgattagtgggaaacatgaaatattgggtggaaaatgcttatcatattgcctgacacttggtaaatgcttctaaatggatgtcatcattactgcattttactgaatttaagcaactcacgactctaagatgcatcattactttatgtgcctctaagaaagaaagaaaaactactgccagttaaactatgacactatttgcttgcgaagtgcatccagatttcagagatgtcagtacgtgaaagaatgggcagtgtggtatttcacttctcttttctcaactctatgaagattcttactttactgaattagacacttaacagtacatgaaaattcctgaaggagaagtatacttagtgtgtttgaggaacaacaaggaggccagtgtggctgatggggaaagtggggtaaggacaaggtccaagacagccaggggatgtctgattccatgtcttcactattgtgaatagtgtgaacagaagagtttcatgatatgactttcatttcttttatgactacatagtattccatgctgtatatgtaccacattttcttttcttttctcctttgctttttctttctttcttttttttcgtttctttttttttattacagttttattttttatttattttttttggaataatttttttttttattatactttaagttttagggtacatgtgcacattgtgcaggttagttacatatgtatacatgtgccatgctggtgcgctgcacccactaacgtgtcatctagcattaggtatctctcccaatgctatccctcccccctcccccgaccccaccacagtccccagagtgtgatattccccttcctgtgtccatgtgatctcattgttcaattcccacctatgagtgagaatatgcggtgtttgggtttttgttcttgcaatagtttactgagaatgatgttttccaatttcatccatgtccctacaaaggacatgaactcatcattttttatggctgcatagtattccatggtgtatatgtgccacattttcttaatccagtctaacattgttggacatttgggttggttccaagtctttgctattgtgaataatgccgcaataaacatacgtgtgcatgtgtctttatagcagcatgatttatagtcatttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgattgccattctaactggtgtgagatgatatctcatagtggttttgatttgcatttctctgatggccagtgataatgagcattttttcatgtgttttttggctgcataaatgtcttcttttgagaagtgtccgttcatgacaaacccacagccaatatcatactgaatgggcaaaaactggaagcattccctttgaaaactggcacaagacagggatgccctctctcaccgctcctattcaacattgtgttggaagttctggccagggcaatcaggcaggagaaggaaataaagggtattcaattaggaaaagaggaagtcaaattgtccctgtttgcagacgacatgattgtttatctagaaaaccccattgtctcagcccaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttcaagttcatatggaaccaaaaaagagcccgcatcgccaagtcaatcctaagtcaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttagacctaaaaccataaaaaccctagaagaaaacctaggcattaccattcaggacataggcgtgggcaagggcttcatgtccaaaacaccaaaagcaatggcaacaaaagccaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacgatgaactcaaacaaatttacaagaaaaaaacaaacaaccccatcaaaaagtgggcaaaggacatgaacagacacttctcgtttctttttttttgagatggagtctctctctgtcgcccaggctggagtgcagtggtgtgatcactgcacctccgcctcccagattcaagcaattttcctgcttcagcctcctgagtaactgggattacatgggcacaccaccatgcctggctaatttttgtatttttgatggagacagggttttaccatgttggccagtctggtctcaaactcctgacctcaagtgatccgcccacctgggctgcccaaagtgctgggattacaggcgtgagccaccatgcctggcccacattttctttatcgaatccactattgatgggcacctaggctgatttaatgtctttgctattgtgaatagtgtgagcggaagaatttcatgatttgatttacattctaaaagatacatgggaaaaggtggacaaagggagatcagttaggaaattattgcactattcctggcaatggaagacagtgaattagcttattatgatagtaaaatatgtgagaagaagtaggtgggatatgggaatatttttagggtagccagtagaatcttctctcagtggatggactgaatacagggtattgaaggagtcaagaatggcctcaagatttttggcttgagtcgttgaaagaatggaacctgaaaaaaaaaaaaaagactgaaggatgagctttggagggagtctgtattttactcaatgttatctttattccttatttacatctatctctaaagtttacctctttttttaaataaatatttttaaatacctattttctccccaatatgcactgagaaataaaaatgaataagatacagtggctttattcccctcaaaattttgccacctgcaggggagataggactcacacgtggtacattacaaagcagtatgtatctaatagtataagaaagatatttaaaatgtgagaaatcagagaaaggaggcattcattgtaattggccctatcctcccctcaaaatgcttgcttttgttgagtacaagttcattacattggaaaaggagaatgctagcataataactttaactagttttttcactttgggtaaactgcctccttgctgtacctcaaaccacctccacaacaagcatgctttttcctcaagtttacctttgcatttgctgttccccttgcataaaccatcatccctgcaagagtcacctggttgctgtcttccttcactcaggtctctgctccgagtcaccttatctaagaagcctaccttgatttctttatataaaagaatgcctattccttttgcatactttttcttcacagcacttgccatcttctctcacattatatatttatttgctttattggctgtttccccacatgagaatgtaaactcagtgacaaaaggcactttgttttgtttaccactgtattcccgcaaggccaatgaattgcttaacgaattatttaataaatataggaattaaaataaaccatgggtttggaaagcaataagcaaatggggttggagataggactggactctgaacacagcgttggtttcagaagagagagaagacactagagaacagctctctgactaagctacatgcctctgtggcagcctggaatcttgggacacacggcttgcaccaggccactcttttggttaaagcaaaggttaaagtctggtgtattgggacaaactttggatagagatacagaatgtgatctacgtcaatccctactacttaatagctgtgggccttctgccagtttcctcatacaggatgagtatcccttatctaaaatgcttgggaccagaagtgtttcagatttcagaattcttggactttggaatataatataatataatataatataacataacataatacaatacaatacaatataatataatataatataatataatataatatagtataatgtaatgtaatgtaatataatataatataatataatataatataatataatataatataatataatataaatttacccaggtgagcatcccaaatccacaaatccaaaatccgaaatgctctaatgagcatttcctttgaacgtcatgctggtgttcaaaaagcatcagattttggattttagatttttggatttgagatactcaacctgtatatattttgagagtaatagtgtgtactctgccaactacatagggttattgcaagggtaaagcaaagataagccatgggacagcactttgcaaactgttaagaattctattatcagggtcaagatggcaatcagggacactatgacatggactacagtcaaaagatccaggaatgcctgccttggacagctagatcttctccccaggaggtgcctgagggagacatacacacaggtgacagtcacaaagggtaagtgaagttctacattattagtagctgggcttgggtcctaaatgccaatgtgagcgttctgataaactgacctgggcatgtagcccaggttaaaggcctccagaagcctctctttattcattaagtcactggtgacaaggactaaaacacttagcacactcagcgacatgtaagatcagtgttgaggagggattagagcaggcccctgagagggccattttctttgtgtgattttcaaaaagtacattgatctcatccattggaaattttggccccattagcatcctgtgttagtgtttcctcatccttactcttcccttcaatttcacttctcagaaagtggaatttgagaatttgttgcttcatccccaaacagttcagatttgcccttaagattcaagcataatgattacctgtggcacacattacctctactggagaaattatacttttgataagaataggcaaagtgatattacattcatatgttcatctcttgattcaaaactgacttggaaatcatagtggatggtgtgttaggctgttcttgcattgttataaatacatacccaagactggtcaatttataagaaaagaggcttaattagctcataattctgcaggctgcacaagcatggcattggaatctgtttggcttctgaggaggcctcagggagcttataatcatcgtggacggtgaagtgggagcaggcatgtcacatggcgaaagcaggagccagacagagagagtggggtggcggggaagggactgcacactcttaaatgaccagatcccctgtgaacttagagcgagagctcacttatcaccaagggcatggcccaagccattcatgagagatcttcccccatgatccaaacaccctacctcctctcccacccaccagcccccacctccagcattggggattacttttcagtatgagatttgggtgggcacaaatatccaaactatatctgatggtgtattaatccattttcatgctgctgataaagacatacccaagactgggaagaaaaaggggtttaattggatttacagttccacatagccggggaggcctcagaatcatggcacgaggcgaaaggcacttcttacatggcagcagcaagagaaaatgaagaagaagcaaaagcagaaacccctgataaacccatcagatctcgtgagacttattcactattacaaggatagcacgggaaagacgggcaggcccccataattcagttacctccccctaggtccctcccacaacatgtgggaactctgggagatacaattcaagttgagacttggtggggacacagccaaaccatatcagatggtgaacatagaagtgttcctgtgggtatatggccatgtttcttgccaaaacttcctgcatatgattctggttaattgcagtttctggaagtatcagctaatccttggcctaaagctatctttcccttttttagggtgagataagactccaggagttctccttggtatgatacaagcagaagggccctcttgccttcagttcttcatggttgtagtggatatcctccttcttcaagcccatgatgttccttagggaaggtagttctgctgtttctgtgccaagcctgggcattatgctcacgtgaaacctttttcccttgatttttgctacttctccagggcacttcctgggaatggggcttggtacctattaggactataaatttgcctctctctgacccctccaagctgagagaagtctcgcctttctccctgccacctctactccaccctgcagtccctttctgagggcaggctttctaggaagttctttgtcttcatgtagtttcttttttcttccctgtaaacataaatgtcccctagggtgaatgaacataaaggcctggccacaggctttgaaaaaggaagtgataatacacaaaaatcttcaaaattatctttctccagagattatttcaaaaacagttttattgaagtacaatagacatacaatagactgcacacattaaaaatgcacaagttggccagttttgacttacatataaacctgtgaaatcatcaccataatcaagataatgaccacatccatcactaccaaaagttgcttgtgtccctttatgattcctctggccgctcctccttcccccaggaaacaactgatctctttctgtcactatagattaacttgtttttgttttctaaagttacatataaatggaagcatacagtatgtgcttttttggtctgactttttttattaagcataattgttttgaaatttatccatgttgttgtagcatgcatcaatagttcattttattgctgaatagcaaggcatgatctagggtaggacaagtgaagtgcctagggttaaaatgcagtcagttccttatgatgataatcataatgatgatgactgtaaaaacatttaatgagacctgctctctgaacaagtttttaagtgtaatataccgtatcgttaagtataggcacaatgttgtacagtggatctctagaacgtattaatcttgtataaccgaactttatatccctcgaatatgaattcctcatttccctttcctcccaattcctggcaaccatcattctactttgtttctatgatttaggccatttttgatatgtaacgtatgtggaatcaggcaatatttacccttctatggctgggttatttcacttagaattatttctaccgggttcaaccatttagtcaaaaatgttaggatttctttctttcttcttttttgttcttttcattattattttttattatcattatactttaagttctggggtacatgtgcataatgtgcaggtttgttacataggtatacacgtgccatggtggtttgctgcacccatcaaaccatcatctacattaggtatttctcctaatgctgtccctcccctagccccccacccaatttctttcttttttaaagctgaataagattccattgtaggtgtatcccacattttctgtatctacttatctgctgatagtcatttgtgttgtttccatgtcttggcttttgtgaatggtgctgcaatgaacatggaagggcagatatctcttcaagatgctgatttagatcctcttgggtatataccagaagtgggactgctggagcatatggtagtcctatttttaattttttgaggagcctccatactgactgcatcattttacattcccactaacagtttataagggttccagtttctctacatccttgccaacatttgttatcttttgattttgttgatgatagtcatcctgacatgtataaggtgatatctcattatggttttaatttgcatttccctaatgattagtgatgttgaacaccttttcgtgtaacttggccatttgtatgtcttctttgaagaaatgtctattcaacttctttgaccactttaaaatcatattttttgtttcctatcaagttgtatgagttccctatatatcttggatattaaccttcaaatatataatttgcaaatattttctctcattccaaaggtcttttcactttgttcattgtttcctttgctgtgcggaagtttttgttgttgttgttcaattgtagttccgcctgtttttgcttttgttgcttatgtgtttggtatcatatccaagaaatcattgcttagaccaatgtttttttcatgtatcttcttccaggagttttatacttttagatatcatgtttaaatctttaatccattttgagttgatttttgggtacaatgtatgataatagtttaattttattatcttgcatgtggatatccagttttcccaacaccatttattaatgagatttttttttttgcccattgtttatttttgacactcttgttgaagatcagttgacctgtgtgggtgacttccaggctctctattctgtcctattggtctatatgtctgttttttcaggtaccatactgttttaagtaactttctaaaatattttgaaattaggacgtgtgatacttctgactgatttcttctttttcaacattgctttggatattttgaaactttttgtggttccatataaattttagggtttaaaattatttcaaaataatttctgtaacaaatgtcactgagattttgaaatgcaatcagttgtgaatccgtttcaattgttcttttttttcttacaatggtttctatttttctgctttgtcaaatgcctgataattttttatagtatgggacacacttacaccttaaaactttttgttagatggctgaataggaacagctccagtctacagctcccagtgtgagcaacgcagaagatgggtgatttctgcatttccagctgaggtactgggttcatctcactgggacttgtcggaaagtgggtgcagcccacagagtgtgagctgaagcagggtggggcatcgcctcacccaggaagtgcaagtggttggggaattccctttcctagccaagggaagctgtgacagatggtacctggaaaatcgggacactcccagcctaatactgcgcttttccaatggtcttagcaaacggcacaccaggagattatatcctgcgcctggcttggagggtcccacgcccacggagccttgctcactgctagcacaccagtccgagatcgaactacaaggtggcagcgaggctgggggaggggcctctgccattgctgaggcttgagtaggtaaacaaagaagccgggaaggtcgaactgggtggtacccatcacagctcaaccaggcctgcctgcctctgtagacgccacctctgtgggcagggcgtagctgaacaaaaggcagcagaaacttctgcagacttaaacgtccctgtctgacagctttgaagagagtggtggttctcccagcatggagtttgagatctgagaatggacagcctgcctcatcaagtgggtccctgacccccgagtagcctaactgggagacacctcccagtaggggcctacagacacctcatacagctgggtgcccctctgagatgaagcttccagaggaaggatcaggcagcaacatttgctgttttgcaatatttgctgttttgcaatatttgctgttctgcagcctgcactggtgatacccaggcaaacagggtctggagtggacctccagcaaactccaacagacctgcagatgagggtcctgactgttagaaggaaaactaacaaagagaaaggaatagcatcaacatcaacaaaaaggacatccacaccaaagccccatctgtaggtcaccatcatcaaagactaaaggtagataaaaccacaaagatggggagaaaccagagcagaaaagctgaaaattctaaaaatcagagcacctcttctcctccaaaggaacacagctcctcaagagcaatggaacaaggatggacggagaatgactttgacaagatgacagaagtaggcttaaaaatatcagtaataacaaacttctccaaggaagtttggaggatgttcaaacccatcgcaaagaagctaaaaaccttgaaaaaagattagacgaatggctaactagaataaacagtgtagagaagaccttaagtgacctggtggagctgaaaaccatggcacgagaactacgtgatgcatgcacaagcttcagtagccaatttgatcaagtggaagttcaagtgaatgaaatgaagcgagaagagaagtttagagaaaaaagagtaaaaaaaaatgaacaaagcctccaagaaatatgagactgtgtgaaaagatcaaatctacatctgactggtgtacctgaaagtgacggggagaatggaaccaagttggaaaacactcttcagggtattatccaggagaacttccccaacctagcaaggcaggccaacattcaaattcaagaaatacagagaacgccacaaagatactcctcgagaagagcaaccccaagacacataattgtcagattcaccaaggttgaaataaaggaaaaaacgttaagggtagccagagagaaaggtcaggttacccacaaagggaagcccatcagcctaatagcggatctctcagcagaaactttacaagccagaagagagtaaaggccaatattcaacgttcttaaagaaaagaatttttaacccagaatttcatatccagccaaactaagcttcataagtgaaggagaaataaaatcctttacagacaagcaaatgctgagagattttgtcaccaccaggcctgccctacaagagctcctgaaggaagtgctaaacatggaaaggaaaaaccagtatcagccactgcaaaaacatgccaaattgtaaacaccatcgatgctaggaagaaactgcatcaactaatgagcaaaataaccagctaacatcataatgacaggatcaaattcacatgtaacaatattaaccttaaatgtaaatgggctaaatgctccaattaaaagacacagactggcaagttggataaagagtcaagacccatcagtgtgctgtattcaggagacccatctcatgtgcagagacacacataggctcaaaataaagggatggaggaagatccaccaagcaaatggaaaacaaaaaaaggcaggggttgcaatcctagtctctgataaaacagactttaaaccagcaaggatcaaaagagacaaagaaggccattacataatggtaaagggatcaattcaacaagaagagctaactatcctaaatatatatgcacccaatacaggagcacccaaattcataaagcaagtccttaaagacctagaaacaaagagacttagactcccacagaatcataatgggagattttaacaccccactgtcaacattagacagatcaacaagacagaaagttaacaaggatatccaggacttgaactcagctctgcaccaagtggacctaatagacatctacagaactctccactacaaatcagcagcatatacattcttctcagcaccacatcatacttattccaaaattgaccacatagttggaagtaaagcactcctcagcaaatgtaaaagaacagaaactataacaaactctgtctcagaccacagtgcaatcaaattagaactcaggattaagaaactcactcaaaactgctcaactacatggaaactgaacaacctgctcctgaatgactactgggtacataatgaaatgaaggcagaaataaagatgttctttgaaaccaatgagaacaaagacacaacataccagaatctctgggacacattcaaagcagtgcatagagggaaatttatagcactaaatgcccacaagagaaagcagaaaagatctaaaattgacactctaacatcacaattaaaagaactagagaagcaagagcaaacacattcaaaagctagcagaaggcaagaaataactaagatcagagcagaactgaaggagatagagacacaaaaaacccttcaaaaaatcaatgaatccaggagctggttttttgaaaagatcaacaaaattgatagaccgctagcaagactaacaaagaagaaaggagagaagaatcaaatagatacaataaaaaatgataaaggagttatcaccactgatcccacagaattacaaactatcatcagagaatactataaacacctctatgcaaataaactagaaaatctagaagaaatggataaattcctggacatatacaccctcccaagactaaaccaggaagaagtcgaatccctgaatagaccaataacaggctctgaaattgcggcaataattaatagcctaccaaccaaaaaaagtccaggaccagatggatgcacagctgaattctaccagaggtacaaagaatagctggtaccattccttctgaaactattccagtcaatagaaatacagggaatcctccctaactcattttataaggccagcatcatcctgaaaccaaagactagcagagacacaacaacaacaaaaaagagaattttagaccaatatccctgatgaacattcatgcaaaaatcctaaataaaatactggcaaactgaatccagcagacaacaaaaagcttatccaccacaatcaagttggcttcatccctgggatgcaaggctgcttcaacatatgcaaatcaataaacctaatccgtcatataaacagaaccaaagacaaaaaccacatgattatctcaatagatgcagaaaaggccttcaacaaaattcaacagcccttcatgctaaaagctctcagtaaactaggtattgatggtaagagctatttatgacaaacgcacagccaatatcatactgaatgggcaaaaactggaagcattccctttgaaaactggcacaagacagggatgccctctctcaccactcctattcaacatagtttggatgttctggccagggcaatcaggcaggagaaagaaataaagggtatttaattaggaaaagaggaagtcaaattgtccctgtttgcagatgacatgattgtatatttagaaaaccccatcatctcagccccaaatctccttaagctgatcagcaacttcagcaaagtctcaggatacaaaatcaatgtgcaaaaaatcacaagcattcctatacaccaataagagacaaacagagagccaaatcatgagtgaactcccattcacaattgcttcaaagagaataaaatacctaggaatccaacttacaagggatgtgaaggacctcttcaaggagaactacaaaccactgctcaacaaagtaaaagaggacacaaacaaatggaagaacattccatgctcgtgggtaggaagaatcaatatcgtggaaatggccatactgcccaaggtaatttatagattcaatgccatcccccatcaagctaccaatgactttcttcacagaattggaaaaaactactttaaagttcatatggaaccaaaaaagagcccgcattgccaagacaatcctaagccaaaagaacaaagctggaggcatcacactacttgacttcaaactatactacaaggctacagtaaccaaaacagcatggtactggtaccaaaacagagatatagaccaatggaacagaacacagccctcagaaataataccacacatctacaaccatctgatctttgacaaacctgacaaaaacaagcaatggggaaaggattccctatttaataaatggtgctgggaaaactggctagccatacgtagaaagctgaaattcgatcccttccttacaccttatacaaaaattaattcaagatggatcaaagacttaaatgttagacccaaaaccataaaaaccctagaagaaaacctaggcaataccattaaggacataggcatgggcaaggacttcatgactaaaacaccaaaaacaatggcaacaaaagccaaaattgacaaatgggatctaattaaactaaagagcttctgcacagcaaaagaaactaccatcagagtgaacaggcaacctacagaataggagaaaatttttgcaatgtacccatctgacaaagggctaatatccagagtgtacaaagaacttaaacatttataagaaaaaaaatcaaacaaccccatcaaaaaatgggcaaagaatatgaatagaaatgtctcaaaagaagatatttatgcagccaacagatacatgaaaaaatgctcatcaccactggtcatcagagaaatgcaaatcaaaaccacaatgagataccatctcacaccagttagaatggcaatcattaaaaagtcaggaaacaacaggtgctggagaggatgtggagaaataggaatgcttttacgctgttggtgggagtgtaaactagttaaaccattgtggaagacagtgtggcgattcctcaaagatctagaagtagaaataccatttgacccagcaatcccattactgggtatacacccaaaggattataaatcatgctgctataaagacacatgcacatgtatgtttattgtggcgctattcacaatagcaaagacttggaatcaacccaaatgtccatcaatgatagactggattaagaaaatgtggcacatatacaccatggaatactatgcagccataaaaaaggatgagttcttgtcctttgtagggacatggataaagatagaaaccatcatttgagcaaactatcgcaaggactgaaggccaagcaccgcatgttctcactcatgagtgggaattgaacagtgagaacacctggacacagggcggagaacatcacacactggggcctgtcgtggggtgaggggacgggggaggaatggcattaagagaaatacctattgtaaatgacgagttaatgggtgcagcacaccaacatggcacatgtatatatgtgacaaacctgcacattgtgcacatgtaccctacaacttaaagtataataaaaaaaaaagagaaaagaaaaaaaaaaacttttgttgcttatccgaaatttaaatttaactaggtatactctattttatctggcaatcaggtgtcctgagactctagcttcctttacttctctggactcccatttttgtctcctcaactcaggaaattctccaggctctgcctgagttcccactcattgtgttgtatcctggacacactctcatgccagtaagctgatgccaccgtaggggtcaacttttttgtttcctgactctcagattgttgtccttcgttgtcagatgctcattatcttaaaacataatttttcatatattttgtcctttttggttgtttcacactggagagcaaatctggtctctgttactccgtctttggttgaaagtggaagtctgtatccagaagttattgtttatagccaggagccagaacatggttctaaccagggccaaaaatgtgtgtttgatcccttcataggtgagtgtgtagtttctttatagtttttatgcaattaactactgaaaaaaataattctttgggtcataaaggcgcctggtgattatgagggttcctcactactgtcaggaaattgattagaagtaaacatcttctaataagagaatgggatgggccactggggttattaccaagtacaaagcataaattggcacattacattgtattacattgattgtcagagtttgaaccaaaatatagatttggctgggggtcaaatctcggaggcccaaattgtatttggagtcagggccaggaagacaagtctgatagagtcagggtgatcaaactggggccatagaggaatttaggaagatgtaaattctcttattccttgttcaatattcagtgatattcttcctagggggcttggaaataaaaataaaaccatgtcctctcttttctctttggtttctcttggtctagctatatgccgctatcctctgggcatgtcaggaggccagattccagatgaggacatcacagcttccagtcagtggtcagagtccacagctgccaaatatggaaggtgaggatggttacatcaagaaagcccatgttctggggttgggcagatttcctgggcacagattcctgaccctaggggctggggaaggtggtgttattggcaggaactgttgagaaatgaaggacagacagcaagtgggcctgtcctgctctgtatgttttcagggagagaatttaggacttagccacatgtactggtctggcagccccatctcaaccacagaggccccagggccacattacccacagtgaatgggttcattgataggaagtggatttgctttccctgactttctcattgtgagctgctgcaggaggcaggcaacagtggcacctatgggaaggtgcagattatgtaggacactgggagttagcatccactcctcttctcagtgggtctatgaaacatcactcactgcaaagcatcactcactgccaagcatcaagacactgaggcctccaacaagttctccacctctctcaaggatctcttacacagaagggccatccttgccaacagacagagtgtggcagtttcctggggcgattgatttcaggggtcttgtacatcaataaactccctactgctctactggttggacccaatactctacctggaatcttattattttgtgaagtattgattttattaaaatggaattacccataagaaaatgaatcctgatgaacctttccccttaatttcttagtgtatgaaagactagagtgatggtaggtggaattggaagggagcaacaaatgaatgggtaatatctgtgagaaaagggagcaatccaagggagaggaaaagtggtagggagttctcaaagtaaactttataataatttcacaattttgctcagtggtagctcagcctgaaggtaataaaatgaaaccagattatttactacttccttgacatgctcttccccctcatcctccccatgtgtttctatggctccttgctggtctgcaatgcccatcctgctgcattgcataacccacactggagaccctactatccctcctccctattgccataacttgatgtccaagcaaagggtggctagctttgttaggctgagtttgaaagagggaagggaaatatgaaatgactcatgcgggaacagagagtaggaagagttttatttctctttatgcttctaaatcaggatgggaagtgaaggctatgtgtgatctgaaggcctcagctcactgtctttcagggtctcatggtactccagggaaagggatctgaccacaaatgggtactgtggtggcagtgaaaactgtggcaagaacccaaaatgtttataaggacagcctgctctctcccctcagtacagggcagctgcttgcctgtgaaccagtaaacagctctgtggtttcatggttgctccctctctccccaaccctcacctctcaaggctggactcagaagaaggggatggagcctggtgccctgagattccagtggaacctgatgacctgaaggagtttctgcagattgacttgcacaccctccattttatcactctggtggggacccaggggcgccatgcaggaggtcatggcatcgagtttgcccccatgtacaagatcaattacagtcgggatggcactcgctggatctcttggcggaaccgtcatgggaaacaggtaggaagaagagacatccagatcctggatgtccaagaccatattttgacttaggctaggagaagaagggtacaggttttctgtgtggatatgtgtgtccacagaccaggcatgcccttgtaaatgtgtgacccagggtgagggaggcagggaaggaatatcaaggctgtggggaaagcagagtagatgcattctgctcctcgaattaagaagagagagtccatcaaaaacgtggtggggtgaagaaaagtgagcatgatttaataccacctcttcactcattctcttctctctcctcaggtgctggatggaaatagtaacccctatgacattttcctaaaggacttggagccgcccattgtagccagatttgtccggttcattccagtcaccgaccactccatgaatgtgtgtatgagagtggagctttacggctgtgtctggctaggtaggtcactagcccaggaagcctatagactttattaaaaactccaacttctgggaaatgaaggcaatcaaatcagaaagggatgggatcagttactcctggaatggtgatgccctcttgggaagaatagagacagaagcattgctcattaacccagggcttagggccccacactgagtcatgcattcagagtcattacgttgactgccatggaggggcactcctccaaagtcttccctcggtgtccagatggagtcctgctggacacgctgtgcaagcttatacccttgaaactcacatagttccctgaggtaatgggcctgagcagtaggcactcacttggctgtgtttcctttgcagatggcttggtgtcttacaatgctccagctgggcagcagtttgtactccctggaggttccatcatttatctgaatgattctgtctatgatggagctgttggatacaggtaaatcctgggaaactttattagaatgggaaattggccactaagaaagaataatattttatcatgtcttgggatcaataatcaatcaaccaatcagtatttatttagttcttgtacaaggcatttggaaataatttagatgaaggaatacacatgacctttatttccagggaacttaaagcccggtttgggaagacaagttaattacccagaaaacaatcaattacacaaagtaatggctaaagtttgtgatatggctctaagtataatagccatatttttgaccctcaaaacaggacatgtagtctgacacaaatattgacctacttatggaaacaaagggccagagaattgaagtaataagtataatactctgggcaagtcaagaagtcagaaagaatagaccttatacatataaatgctttgaaatttgtaaaaaggggactagtaagacttacaatagacaaggaaagttctaggttaagatgagaattgtagcagaggaaggtaggtagaaggtagcagaagaaaggaagcagggctgaaggcaagactgacctggctatagtagaagctggagttgagaagaacagaatggaacaatggataagcctgaagtggatcaaaggagtgccatggcaaagaagtgtgaccagatatagtaggaacttagggttgggtacaggatgctcccacctatactgtttcacttcaagcctttaagccttgccattcttctagcccacactaacacttttctctctaagcttggtctgcatctgcaaagtgagcctgggagagaattcagtccatcgtgaagaccagaccagatgatggaggttgacagggctcgagagaaggagatactagtgagtctcaggaagggagtgtggaattacagccttctactggcttaggtgtggagaatgaactattgccctctagctgccatcccctgggtgaagacctgattcatccactcttctactcctagtcacttcctttccatcttcttaatcactgttctttcctttttgcttaaacggtcttggcataatttcgctcagtcacaccccaacaggcatgtattcaaggcacagaggaaatcagagatcaaccgaattcagtttaatttaataagcctttgttaatcaactgttactatatacaaggaatttggccaggcatgttaggtattcaaaggtaatggagtcattgatcctacctttaaagtgctcacaatttggagtaggggctaaaacagaatataagttactatgattcaagtatatgagcaagagatagcctatgactgctctggggttcaaaggaaaaagtaaactatagtaggaaagcacaaaaatggggatgaaaacatctggatttcatataataaaagttacttctgtttgctacaaggcttaagggattaaaatgtgtgtgaaaagcatttcgtaagataaattgctatgcacatataagttgttatctgattatcatttttctgactgaaggaccagtaaagatatggtagaacagatgaaatctgaactgggtcccaaagaatgggtagtactttcacaggtgcagaatcttgaaatggaattttaggcagagggaataatagcatgaggaaaaaagaatcatggttataaagtttaggggactgtctgggagaaatcaacagtttatttaaccacgggccatatttatgtggcaaagtagtgcaggagaagcaaaacaaagcagattatgccatattttgaaggaacctgaagtcaaagccaaaaggagcaagaagtggaggaggaggaagaggagaaattgctgttcttattcttcataaacattatcattaatgtaatgagtcattgaagatttttaaagctgaggaaggacatgattggagctgtgctttagacagattaatcaagcagcatgcaaaggttagattggaatggaatgaggaggctatgaggaacattttaaaactcttagttctgccaagtaaaaagaattaatgaccaaggccataaccgtgactgtggggaatgaggaaaagatattcatttaggatgtttgaaatctgattggatgtatgaggcaaaacagtagggtgagaaaaagggtcagatgaagtttgacagtctaagtgactgaaacaatggggatgacatttatttattcgacaaatatattttgagtgcttaccttgcgccaagcattttccattactatgaagagaagtataatttatggaaaagggaaaccaaggagaaagacttagttcagtaggggatgatgatggatttagtttggagtgtaggtctgtgagactgagaagacattcagtagagaaataaggaaggtagccggaattatggctcttaactccaggggaaagataaaaaaaattacaaaaatgtagacatcattggctttcatttaataattgaagtaatgaaatatctggtggaggtggctgagtgaagagatcctctcagccactgagagatgacatcagagtctcaggaaatagtcatattagcaaaagttactttttaaatttaaaacaatttcaagaataatatatgcttactggtgattttaataactctaaagtatataaatattgaacatttattttcttatcccttcttcacttgatttaattttgaccaagggggaactgaacaggcattccttttaatttctgttgcttttcataaaactttcctgggatttatttttctgaggataatgggaaatatatttattgcaagtaaacttacgttctcctagctttctgatctgtagagagaagaaaaagatggcaaaaccttggaagggagatccaggctctggtcactgtggataaggccagtcaaattttataatttaccaaacattttcaatgattaaatatattttaaaaggctggcataatcacccttagtatttttttattttttgcgattcacaaattttacatattattgatctagctctaaacctcaagggtcacaccaattattgcttgagggtgagtatcccaatctcgtcatttgggaaatttgtgtcatatctgccagagcttgagatgcagagcttgaggaaagagaagttccacatgatggtttttgctctgtccctccataaaaagaaggaaggagcctcttcgtgttctctaagtagtcaaataagcatacatgcagttatattccatccagcagtcaggcttctggaaaactcagcctttaaaacagagcctcttactcagaagtcaaaaaagcctgctaagctagaacacaaaactaaatccaatacctgggattctcatgaaatccttgcatcttacttcctgggtctagtatcctacaaatttaattagaattaaatagaaattattatttaaatttagagcttgtattttcccccttagtttaatatcaaactgggttgttggatatgtggaatagaagagagtgtgactggattctctgaaattgattcactgaacaatgacaatatgtagttaaaggagagtcagcaccaagctctggaaacatttatatgatagtggggcaagtgctactatgtatggtagcattcattctatgcatttatccatttcctcatttactctatgaattagttcatcaaatgcatttgataatattaagtcattatgtgaagacatattcagtatataagaataggcaaggtgcaataaattatagaaagcttttcatattaaataagacattgtttaaaagttttactactaaaggagtaagcatcaaatattttataaaaattactaatatgaaattcttagttctccattgatgcaagataaacgagtcaatactataatgcaaatctgattatttacaatccttcaattccaagataataagctcatacaaaatctggagtgaagatgccgggtaaaagctcttccacgaatgtgtggttaactcagatttctctctccttttcctcctcttctcctggcctgagcagcatgacagaagggctaggccaattgaccgatggtgtgtctggcctggacgatttcacccagacccatgaataccacgtgtggcccggctatgactatgtgggctggcggaacgagagtgccaccaatggctacattgagatcatgtttgaatttgaccgcatcaggaatttcactaccatgaaggtcagtggggtcgggtgtggtggaacttctttaaggaggcacaaatcatagtgtggtagagaaaaggcatgctagaaaaaagtctaggctgatgccagttcaatgacatattctgttactaactagctaaatgcatttcacaagtcacttcatctctaagggcgtgattctccatttgtgtgtaagggagttagaataggtcagtgagctctcttttttttgttgttttgacactaaaggtgacatatccattaagccaataacacagtgtccagggcccacagatttttaagacgcataaaaatgctctaatttcttttaatatcaggtgaaaaaaataaatttttagggtcaaagattatctttttaccagcacagttgtgtgtgtgtgtgtgtgtgtgtatacacacacaactatatatatatacacaatgatatatatatatacacaactacatatatataaccatatatacatatacaactatatatatacatatacaactatatatgtatatacatatacaactatatatatgtatatacatatacaactagatatatgtatatacatatacaactagatatatgtatatacatatacaactagatatatgtatatacatatacaactagatatatatatgtagttggtgttctgcagtcttctgaatttttccttctaggaacctcacagtggcagtgatagttggtcaggccttataaaacctgggctgcaaccctggttacctgtatgcactgtctgcccttggccacctcacttcatgttgctggactgcagttttctcatctgttaaatgaggactgcaccagcatcttctcagcccttccttgccttgaactctctcgagtctataatcctctcagccgcagttgccgtctaccctgattcgtgtgtgtatctatacacatagatacatacacacatatgtgagaatatgtgtattctcatttagaaatgagatttctaaaaaaaaaaatgaaattgttttagattgtacactattttgggtttctcacctagccctgtgaaaagaagaagactggctctgaaatgtacctaggaggaagcaggatggcagtcttcctcttacctcagttcagaatagctcgaatcagggtagacggcaactactgagttggctggcactgtgtctccatgcaccttagcagggccaacctatcactcacatgcctctttctctaccaggtccactgcaacaacatgtttgctaaaggtgtgaagatctttaaggaggtacagtgctacttccgctctgaagccagtgagtgggaacctaatgccatttccttcccccttgtcctggatgacgtcaaccccagtgctcggtttgtcacggtgcctctccaccaccgaatggccagtgccatcaagtgtcaataccattttgcagatacctggatgatgttcagtgagatcaccttccaatcaggtagggagctcaggtcctctttgggaagtgggagcaaggtgatgaaggaggaatgcacatgcccagaacttctcattagcaggtctctgagaggagtgggattgtacaggcagcttctcattgacggatacggtgaatcctttgtgtgactagttttaactgtggaggggatgggatgagaatggaagtgaacaaaatatgcacgtaactcttcttagaaagaatgtttttaaatgagagtacagtgtgttaaaaattttatataatttaaggtagttaacgagtgcgctgttaaggaaaagcacacctaagcattactgagatatttaaatgaaaatttctaaatatacacaaaagtaaagagaacaataaaccactcatcactcagctttactaatcattgatattttgccatatttgtttcacccattccttccttttggctgtagcattttaaagcaaatatgagatactacttgatttcattagcaaataattggactgcatctaagaaaatagcatcattttcataggtataatgccatgataacatctaacaacctttattattaaataatttcttaataccacccaatatcctgtccatattcaagtatgactaactgtccatcaatgtcttaattcagttgactttatcaaataaggatcaaaataaggatctgttggtgagtttctttattctatttgctctgtgtctcttttaatctaaaaaattccttccatttttatttctttttaaatggattctggcttggctgatggctttctgtggtgtcattttacttgagcttctgttctctgtcattcctaatgactggtgcttacatctacaggttaatttgattccagttcaatatttatttttgtcaagaattctttataagcattctatatatctcagtaatgctgtatattttatgctgaacttcatcatggggatgtaatgctagatcctctcactttttgtgatactaagattgatttctggttcctcatgaactttttccctaagttttatcctccatcaataaccactgtctgaattatttatttcactagggttgctaaatgacgattttctaattcttttattttttctgcatttcttagctgaatttcttccgaatagagtaactattcttcatcatgcaccagtttactcagaaatcacattgaaagagcagcatacgtgacttatcttcccctttaattgttaattttcagagtaatgacttgataccgtagcaaccctcaataatgtctaattaattttggtttttacccttatttttttggtatcattataaactcattgctttttatcaatttaatgcttaatcgattgcagttattattattagtagtagtatgtttatttttagacagagtctcgctctgtcacccaggctggaatgcagtggtgtgatcttggctcactgcaacccccgcctcccgggatcgagtgattcttctgcctcagcctcccaagtagctgggattacaggtgcctgccactgcgcctggctagtttttctatgtttagtagagatagggtttcgccatgttggccaggctggtctcgaactcctgacctcaagtgatccgcctgccttggcctcccaaagtgctgggattacaggcatgagccaccgtgtctggccattattctttttaatactcaagttgtgctgtctttgactagagtttgcatttttttttttttgagacggtatcttgctctgttgcccaggctggagtactatcttgagtcattgcaacctccacctcccaggttcaagtgattctcctgcctcagcttcccaaatagctgggattacaggtgcctgctaccacgcccggcttttttttttttttttttttttttttttttttttgcatttttagtaaagacggggtttcaccatgctggccaggctggtctcgaactcccaacctcaggcgatccagccgcctaggcctcccaaagtgctgggattacagatgtgagccactgcacccagcctagagtttgcatttttaagggggctctttgctcttttgatctgaccctagaagcctttgataactttcttgttttccgaaagaacaaggtatttcaagctcattttgaacatgtacagcacccgacgtggaatcaaccatttcaagaaatcttgattccttttagtaggaaatggtatttagaaaacacaaactgggtagtagggatgctcattactaataggttgtcattgtttcttggatttttcggtggaagaaagcaatgaaatatgtatatttaaaaagaaagattgtgagtttacattgttacttccaattcaaattaaagataaggtttttatttaaagttgattttatacgttcatctctattatttatgctaaatatcatgtttcctaacaatgttaacagcattttcttcatcctattattcaaaaagcaataccaatattattactagcaacgtaactattgaacattaagatttcttagcaattctcttttctttgtcctgagaatatatcctactaagaatgtgcagtcaaagcactatgttctaaggtcagttgggtaacagtgggataattcttttctgtatatagttatatcaccagtgtggtatacagttagggtaacagtgggataattcttttctgtatgtagttatgtcaccaatgtgatatacagttaggttcattgtattccatttgtttcaacctttagggattccttttaaactttaaacactacttttgattgaacagtaactgcagcaaatagtgatttaacttactacagccagaaactgtgccaaatcttgaaaagttaaaaggtgtttagaacattgttcataccattaaatttaaatgggaagatgaaagatacagaatgctttaaaaaaaaaaaaaaaaaaagaaatgtccaaagcaatatgaacaataaatggtcaggaggagagaggcactagcgtgggtttgtcatagacagcatcaaaggaatgagacttgtactgggcctggaagaaaagtccaggtgtaaaataacataaaagagaggaagagactaatttaaagataaaccacactttgggaggccaaggcaggaggattgcttgtattcaagagtttgagactagcctaggcaacatggcgaaatcctgtctctataaaaaacacaaaaattagctgggtgtggtggcacgtgcttgcagtcccagctacttgggaggctgaggtgggaggatcacctgagccctggaggcagagcttgcagtgggtcgagattgcaccactgcactccagcctgggtgacagagtgagaccctgtttaaaaaaaaaaaaaaagaaagaaaagaaaaataaatcagaagaacccatcctttcagatttgatttttaagggaaaaaaatttcaagatggtttgcttattaagatgtactgataagaaaatagagcatatttttaggaaaacaggtttggtgtttaaatgccttctgtgctaatgatttaatctaagagtaagttgattgattgtttcatctgaatactgagagtagaaatcaagatgctcaaagtcagtgccacttacactgtaagtgttaactgggggacaaatagattcccacccatcctcagggattctcctggcatagtaactggtacacttattatagctaattatagctatgaaccatggtttttactcctggggtctcttgagctttaaatccaatgcataataatattagttttcaccggagcctgacagccgaactccgaaagcacatgcagaggccagatacctccttatttatataataatattaagaagaagaaaaattaggatacctggaagaaatgtctcttagaaaggaaatacatattctaattcttgacccccttgattagacaaatcagagggaaaaactctggcaaagctgccaacatcaatctggcaaatacgcttttgtggtttagtctcaaaagctgtaaaatcacagaaaaagacactccaatgtcagtaaatatctataaagattaagccttaattcataaattgaagggctaggccgatgaggcatagtggagactcttttataaacaatctaatatccttgaaggaaaaagcatcttaacactatgtctgtcccttgcccatttacccgctatattctgtctatattttcactgaatttgggtcctgcttcttatgctttatgacaatgtgtggtacagccctttgaatcactgtttcttaaacttttttgtaccatagcatctagtatgtaatatgtgaaacaaaaattttacaaaggaaaacttgcttttactatttagataaatggtgattttttattcaacgtatttatgattttatctttttaaaagactgatgacaactcactaaattgatcttgtaatgtgctaggtcacaatatgccttcagagaaaacactagctgtctgtcttctccctggctctgactcacccttgttttataacagatgctgcaatgtacaacaactctgaagccctgcccacctctcctatggcacccacaacctatggtatatgtgattcctaattacacaaattaatttgaagggacttactgggggatgaagaagggtggagagttgcaaagccctggctgtgtgggaggctttacaccagttggctttggatgaagagtagccctggaaaggtaggactgtctacctccaagttatggatccaaatatactctcttcactatggattcatgtgcttcttcagctgaggcagagtctggagttgtcctaatttctccatgaaagtttgggaatttggggtcagaaacagggaaagggacattcctgggtcagacccatagtgggtatttctaaggaggggtggaaatcaaagaaacaaaatgctgatatctccaattggagataaatatgccatgtatgcattgctttaggcagtcattggtgacactagccagcaatgtgggaaacatgtgggattgaatgtgtccaaagaggactttcagctaccttggcatgtctcaaagaatgcagagagtgaaactggttcctcacctgtttttcatgaggtgtgattaagggactacagactcaggccataataatacagagacaaaattttgggaagaatagaaaaactcaccattactctgtctgacacatggagagggggagactactgtcttatggaagagggaaataggataggggaacaaagtaataaacagattgggcgtggtggctcacacctgtaatcccagcactttgggaggcagaggcaggcagatcatgaggtcaggagttcgagaccagactggccaacatggagaaaccccatctctactaaagatacaaaaattagccgggcgtagtggcgcatgcctgtaatcccagctactcaggaggctgaggcagaattgcttgaacctgggagggggaggtttcagtgagccaagatcatgccattgcactccatcctgagggacagaacaagactctatctcaggaaaaaaaaaaagtaataaaacagtagcaagaaaacaaaacagtagcaagagcaagaataagcagaagtattaaggtggcatcttccataattatcctcaaggaacagggtctacctccatgtttccagttcagtccctcatacttttacttgacctgtgagatgattgtattctctgccttctctccctggtcacagatccaatgcttaaagttgatgacagcaacactcggatcctgattggctgcttggtggccatcatctttatcctcctggccatcattgtcatcatcctctggaggcagttctggcagaaaatgctggagaaggtgaggaggtgcagaatggtcatgatataagaaactgctcctttctttcttaagccactgttgtcccaggagtaaaaggcaaattgcaaacatttattaatggttggaattaactgaggctaccaagaaacataggaatgaagccagaatagaattagctaagtctattttctcatttttagagtggagatgttggaaccttcctaacgttcctcggaaatgcagaaaactcttcttaatacattgaatctccattcaggcattttccttgaagattctgattcactggattttgggtagagtccaggcatccatatttaaaaaaaaatttaacaagtgcttctgatgcacattcgaggttaagaattactctcctaatgccaagctgaaatttctgataatgctgttgaagttccttctcatctttttcactttgcttgtctgtttggtgtgtgtgtgtgtgtgtgtgtgtgtagaaaaacatccagcttcattaatattcaagccaagtttctcactattctatttctacttttgtgtacttgaaggttgtgattatgtatccctctgagtccctcttcagactctgtctatgccaaatgatctgaataactttttacattgtctaacactttacaggaagcagaatttttcctcatgcataattttatatacacctcattgagtaataactcataggcattgttatgtttaactatttttctgatgagaaaatgtgatgtgcttaaggtcatacaactgatgtccagtcttctatgctttttccaattgttaatattgaaatgcattgatttctttcttcaaaactatattcatttcatcaaagatggaatatctatgacttgtcttggtttcttggaccaaatagcaactcccttggaccaaatagcaactcactgctgcttagtgtctgattcttttggggcctgctgacgactaagtgtttctgcctttggccatgatcacctctcctggtcaattagttcctctcaatagacatggactagtagaggccacaggaactctgagctgtggggctttgaactgcctcctgagggcagggaataggaaggaggagaaatcctgattgatttctgtgtttgggggtggggactcagaggagaccaatgcctgtgtggaacctctattccagaggagaggaaagggcaccatgagaatgggcttgagtctctagagctttctagatgcaggatgggagaaaaatggctcaaatcaggggtaaggaggagcaatcctgatgagtagcctgcaattccttctgatctgtgccaccctcaggttcctgagatggttgatgtttgttttaaggtttttaggaaggaatatttcatggaagagaaggtctccttgcttcccacaagtatgtctaccagtctgaaaacacttaaagctttcaattcgcaaatagcaacagaagactgaaatacagaatccacagagactcactcgctgtcacgagtatgtctcaaggcatgaatgaggcaaatatcaaaatttttcctcctttagaatgctggaaaacactagtgtttaatgaggtattccagagtgagggtggtggtgaactgaaggttcattgaactgcttgaagaggatatgtgaccttcttaaatagtctgttaggaccacattttagccaagggggaaagtctgtgttctctgctatatgagctctttttctctttatttgagccacaatttaccatacttctctgagtctttattctgaggcctgttggaatatcttcatgggctaagagagaaatgttggaatctccaggtaaaaggtagagcaaacattttgcccttgaatatttaacttggtcctccaagaaattctacccctgtacatcctgagtaagatgactcaaggtgctctggaatgacacccagtgtttcttcctgggaacaaaattttatgtggaaaatcattaagtacacacatacattctaagaacattacttaacatagctgtagactgtatgtagcattatttgtaggataattttggtggtgtcctcaacattccttctttgagagatttaacccacatatattgagtacctactttgttctgagcttcatgcttagtactggggatattactgcaacaaaagaccttcagtctagtggaaaagtgcacatctaagtttcgccattgagcaccaaagctgtgtttgttggagtggagcattttacatgtttaaacccaaatagtcacatacatgcaaatgctggcatttgccttttgatggagcaggcaggttatattagataagataccacagaagtagttgctttgttgttctccctcccttattaggtgtaagtcacattgttatctctggacacccagggctttcctgtcccatgttgtagccattttatttttggttagtgaaaatatgggtcagtggtgggtattatcctaatctagggtatcaaaatgatcaagtagaactactgattcatcatgttatagcacatgcattaagttaataaacaatttatgtctattccaaaaccaagtagttaaagggtaagccaggaaaacccatcatttaataaaacatagaaaactctataagattgagatctttggctactgatgggttcttatagcaacagggagataatcttttttttttaagtgtttcagaaagcacatcatagaaattagaagatcaaattctaccttgaagaaatgaaggattattctaagttcctacaacacaggcatttgtggaaccagactgcattcctagcacgtgcagtgttgctggggttaaacagtagtaaagagttatttcatttgagtgggagagctgagtttaagaagaggaggcattgaccatctcttttgtgccaacatgcctttctccttgctcttctcttccaggcttctcggaggatgctggatgatgaaatgacagtcagcctttccctgccaagtgattctagcatgttcaacaataaccgctcctcatcacctagtgaacaagggtccaactcgacttacgatcgcatctttccccttcgccctgactaccaggagccatccaggctgatacgaaaactcccagaatttgctccaggggaggaggagtcaggtgaggatgatgtggtgggcagggtgtcaagggagaaacctcagcaagcatcaggtaggtatgacacgcctgctcccagttcattgcatctatttttagtctctgctaacctcctgagaagtccacattctctttctagatttggtctgccgctgctcctggcctaatttgagcaactctcctttgctactgaatataagatgtgtggagggacccacataccagaaaagaaagaaaaaaataataaaacatgtgaattaggtgttgacaccagaagaacctaaacagtccaaccagtctaacctcattttacatgtcgggaaagtgagaaagtccagatagagtactcacccaacaccaaatttcatgagtgggtaaaggcaggactagatttttacctggagaaataaataaataaatgaagctaaagacattccaaaattcccagtggtgtagccatttaaataactaaaataaataactacttgagttatctattttttgtgaatttctctttggtaaacgtttattagagtaatatgagattttattatgagagtaaaacatttctcaccttttctcaataaaagcattgaaataaaacaataaaacaggtaaaacagtaagtagtgtcagttgattcaaatttcgatgtctttcaaaaaatgatgcagagtcatatcagaagggaagctggagtcacctctttagctcagagcaacttttctataagatcatcaacctgagggacccgagtcagtagaaaagaacttgtagtgttcaggagagaggagctaggcctgagctggatgtagaagcagacaggactaaattcaagagcataggattttataggaaggagcctgaaggggtcttttcaaaggataaaaggaaacttgcatctattttattattcaatggatagaagacatgaaagagcatgctaaattcaagtaatttcacagagacagcaagaataaaggatataaactaatttatttcttggggtgaatggaagtaatttacctatttcctaagtctaaaacatcccccacttcatattttaaggcttttttaagttagaatatgtcttaagctagatcatatgtctttaatctgaaagttttttatacctaaaatcagtttttaagtcaataacagtgtcttaaaattaaggaaataagttattaaaccttcaacctttaatattcccaccctgatataccatgagccttcctatagcttcaccacacaattccacaagtttagccacagaatttgttaatgatcatgtattatgaggaaaagaatgagaaataaaatgatgttctacattctgtaggccacaagaaaatctctagaggaagcaaaaccattttccaagtaagaatgtcatgaagcaggttaggctttcacaggggcatgttttagccctcctctcagagttccttcctgaagagatctccaaagacactccacggaatgagggctcgttgcccttgtcttcccaggctgcagcggtgttgtgaagccagtccagcccagtggccctgagggggtgccccactatgcagaggctgacatagtgaacctccaaggagtgacaggaggcaacacatactcagtgcctgccgtcaccatggacctgctctcaggaaaagatgtggctgtggaggagttccccaggaaactcctaactttcaaagagaagctgggagaaggacagtttggggaggtgagttgattctttgattcccttatagctcgaagaaggtggttggataaaaatgatcagtagaacaaagagtcccttccagaggtggattcacaacagaatgtctcttccatttgtctctccttgcattgtcctctggatttcagtattgggtcaacctaatctttgtcaaatagctgtggagttcctgggacttctgatttctatataggagtttttaaggcaggcaagtcactagacttcagatatcttgaaaaacattcaatattttcaaaaatgtttacaatagattatggagggtgggatccagaagatgtgatcatcttttatgaactgtccaggaacattctgctactcaatagccactttgtttttttcccctttaatgggagttatatgtaaaatggtcaaaaacaacctcgcccctgcctttggtcccagaatcagccactagggaatgaatcaccaactaagataaggaggagtactcttttcatttgccctcagagtgatgcatgcattattaaacttttcattcttgcatcagtcacatgaaccttctgggagctcaaagcaatgttttgaattctaataggaactcttcaccactgccgtctaaactccaaataaaacatttggtttcctaagacatgttattggttttcagtgtggatctcatgttctctccttagataggtctggtgttgcaagaggttgagggacccatgttatctgttcatggtgtgacatgtactcattctgtcccctgggttgaaaacccacatctgttatactgttactaatagacaggagtttcttataatgctaaaccctatactaagggattattttaggaaattgccttacgttttacatgatccagtgactaatgaatcgaagttcaagcatttgtaataaaagacattttatattgggctatgctgtaatataatattgttattgattcattcattcacttagtgttccaagcaatgtgtatttctaaggcaggagagtggcacattagctgggtactttaccattgttttaaatcttatcaattatttgaatcatagatatgttacacatatcttcttattggtcttttgatcattttgcctgagttgtaagagttagtttatctccaggaaatgcccagcaagagtactgagacatcttcaggagaaatgatgatgctgagactagatgacttttgtctaggttcatctctgtgaagtggagggaatggaaaaattcaaagacaaagattttgccctagatgtcagtgccaaccagcctgtcctggtggctgtgaaaatgctccgagcagatgccaacaagaatgccaggtctgtggtctacattttgaattttcctttaggtatttcatctttaggaccaggaatactcctggatacttctgagcaattttttcttttgagatgggaaggtcagtgagctgcctcccatttccactgttatcctttttcttattccgatggggtcggcagtcctcatctttttctccacctctctcaaacacaaaggtacattttccattgtacttgccacatatttacttcctaaccccctgtgcacaagcactgtacttgttcccaacatatacaactgtctaaatgtgtacttctttggtctttatacacagacatactcctgcatgtcattacctcctcttgacaaagtcttcagaatcacacatgactttcttgctaagcctcttccagtctccttttttcacatttctatttattttctcatcccttttttctcttttatgctccttttcttctatttgagcaaccctcaaaatgagaagctacctctatttggaaaattatctatgaacctcaaagcaaaaggatccacagttctctagcagttttccaaactggctaataatctcttggatctttttctacataaattgcagtttccagccacacttcattgtctgacagcctttccattacaataatggtgagttaatcctcacccagagagacaagctggttgaaagctggctgtatgtgtcccagagaaatctccacacctggataacatatgcattttcccatttgtgttgtcttgatttgtctcactgatattcatagtgaaaaaaagtttattattcttattttccaaattatcataaattccaagaatggtgatagacttcattaaaagtcttgcaatggacaagatgtaagataaagaagagagaaaggtgaggaaatgaaactggaggctttagaaatttaactgataatttcaatctttaagatactggtaagaataaaaagtgacattctctgggtcttatagagagacgaagtatttcttatattaatcttagcacatgattggagaaagtggcatgaggggaaacaaaaccaaaccaaaccacaggacataggatatgacaaagcctttctccgttgttccgatcggtgaagtttgccattcctccacctgtgtccttagcaatgatggatggccatgactgcaaaccctcccaagccatggaaacccctggtttgagacggaagggggaatgaaagaggggaggtgttgcatcaggacggcattgattccagtggtttatattgacgtagtatgacttccagtccaggattcctagcaaactcttggagggcttataactcatctgctccaacagttttgggaatgtgggctagaggcctcactaacaaaatcttgctactttatgacccaagggtgtctgtcaccatgtactcaaacaattattagtcttggtcaaatgaatacttcttttgacatgattaccaccacagcctttaaagagctctgttttcgacaggctactaaatacatgttgcaactgactgatggttcgtactgcattaagaaaagtcagtatgagaaaataaagagtaatcagataagccaaagatggttgttcacatcatacaaagtttcttcactagtaatagagtgactccatggctttaaaaaaaaaaaggtaaatatcctcgtgcacttaagatatgattttcagtgaaacattttactctgtagagtggtttttagcagcctatttttgattcagcaaatattaatcactttctgtgtggcagggattttgttagatgtgagggatagaaatgtgaataagtcatggatcttgttctcaaaaacattacaattgaataagggcccatgatatttcaggaggaatagaatgagacagatccaggtgtcaatttctctgtcctctcaaaagttatccaaagggaaacaagatttggcataatgtccaggaataggccttggtgtgcattcttctctctcttgattctgactctggcaacttctgagtttatctatgtctgtatcctcccaaggaatgattttcttaaggagataaagatcatgtctcggctcaaggacccaaacatcatccatctattagctgtgtgtatcactgatgaccctctctgtatgatcactgaatacatggagaatggagatctcaatcagtttctttcccgccacgagccccctaattcttcctccagcgatgtacgcactgtcaggtaaacaagccaggtcttccttctcctccctgtggtcatgagagtaacctgggatctgaaaatagggagcagtgagccttaaagtgtatcaatgttctgggatggtgggggaagtcagtgtgcagggaataatggagcagctgccctttggggaaacgcaagggattcatcaagagtagagaaagaatgttgagctttcaaccctagtttgttgataccattttagaatgtgtacctttcacatcttcctgtttccataggctaccttctgtcttcttgtctatttcctcagttacaccaatctgaagtttatggctacccaaattgcctctggcatgaagtacctttcctctcttaattttgttcaccgagatctggccacacgaaactgtttagtgggtaagaactacacaatcaagatagctgactttggaatgagcaggaacctgtacagtggtgactattaccggatccagggccgggcagtgctccctatccgctggatgtcttgggagagtatcttgctggtaagttctcagcattttaaagccctgtctaacaactggcttgtgggctcacatgcatgacacgtggagacaaacctgagacagcaggaggcaaactcaatagactgtagtattttctctgatttgaaattattttctttgtgtactaattcttattatttaatgcttgcctatttctgctaaaaaattaacacacataaaaatttagggagagaagttaaaaagtataagagatttattatctttgtttctctttatcatttctacctagtgattgttcttaatttaagatattaaaaaccttatcatttaaagaaatgttacctatacaaaatattttcttctccattagtttcataagcatgtttctcataattacatagactaattcctataattgtatttcaatacttttcaacatgttctttttaagttcatcattctttgctttataatttttatttgattcttgatattccataccataagtgagtaatttttcccaaatatctctattagaggtatattttctgtccatgcatacctgagactttttattgttgattttataaccactcagctgatttatagactcattagactatacacttttacttcctacaatgccatgtacatgttattttatcttttttcttgtgtgacattgcagagaaaagtctgagaccaatttggttttgttcatttatgattaatctgcttatttcctgctaatattcttataggatttgtaaattaattttgaaataaaaaatgtatagttattcccttgcactcaccaggataaccactatcaatactttggtatattttcttagaggatttttttctatacatatattttttctatgcatgcaattttttttctgtgcatgcatttatatacatatgtacatatatgtaatttatattacataattgggatgacactatgccatacaattctctatctctatttttaacttaacattttattatctgcataatattccatgtgtagatatatactataacttaataatgcctctataatttgatatttaagtactttccaatttcctgctattatgaatgatgctgtgatgtacaccagtttacataaatccttataaagatctttttcacctatatatgtgaattgctatattttacccatttctaatgggatagttaggcattactctccctaaaggtattatatggtgttcaaaacaatatattagactgacattcagaggaccttggttcaaatccagtttttccactactaatggtcttattaaccagaagaatttgggaaacctctcctgtttcatttttctgatctaaaaaataaggataactggcatggtggtatgtacctgtagtcccagctactcaggaggctgaggcaggaggatgaaaatctgggctgcgctgcgctatgacaattgggtgtcctcactaagtctggcattaatatggtcacccctaggagcagtggaccaccaggttgcctaaagagggatgagccagcccaggtcagaaacagaggaggtcaaaactcccgtgttgatcagtaatgagatcaaacgtgtgactggccattgcactccaggctgggcaacataatgagaccccatttctcaaaggaaaaaatatttaataataaataatatcatccacttcacagtgtggttgtctgtattgagtagaaaatgtgagtagtcaatgcatgctatttacacctgaatctcattgctgtcacttgttaaagtctatctttcaacactgtgattatcactgaactggtaaatgagagtcctggattcactgcaggcacttaaactagattcctctatctctaacataaaaaccattgacttcccattcagattttgcctttctcaggagcattttcccaaggctaccctagagttgaaaggttgacctaacactcagttgcagagtcaaatagcttatggctgcgtttttttcagctcttggatctcatccccagagaaacagaaccaagctgagatcatgagaatgaaagaatgggctgtttctaaatactccatgcacatcttggcattttcagaattccttgcctgtggtgggggaaggggtatagctgcagattatgaaatttaacagggtgttgttgtgcacaggttattctgatttcccattcttttctttacttaaatagggcaagttcactacagcaagtgatgtgtgggcctttggggttactttgtgggagactttcaccttttgtcaagaacagccctattcccagctgtcagatgaacaggttattgagaatactggagagttcttccgagaccaagggaggcaggtaagaactgttggggatgaatggatgtggacctgtgtaccttgaaggatggagaatggcaagtcctgccttagcaggagtgggccgagatggaagacagactatccaggtgttagtctttgtcactaactatccagatgatgtgaaagaccagtatcctccccaggcatccatacactttgctttatctatgtgtcaactcattgactcttggagagcataaagggtcatgtcaggtttctccccagctcaaaggtagcaggaaacaaagtcctatctctcatgatttatctatttcttgatctagaccatgtggtttttcttctgggtcaaaaaataaaagtgtaaaagggatgtattagcattcattaaaaatatagcttagtataagaaatggggttggttttgactatctagcaatgtttctagacttggatgtctgaaaatgggcagatatatcctaaacttgttgctcttggaaggcatctctgggcccagtattgtgacatctcaggactcagggaaagatgagttcccaagctccccaggagtctcaattcctagccaagatactccaagcttttttttgggtggcttagatttaaaattcctgtgagatctggaatttaaagcttgccagtaactacagcaccgcacttccccaaaggggcctggcaacagcatacccttggattatggtatctttaccaggaaaaggataatagctgggggaaggatcttgcagacaataaaaagtggatcttctgtggcttgaacagatccaagcaagccaaagatagataaggtattgacaagagataatacattatgcatattaaaaataagagaggggtctgagaatttaattgtatcttgatactctgtaaatattttggcagcagggagtctaaattagctatcttggttatgaggcaatttgccttcttactatagtcaaaggaaaatttactcctgagtacatgagataagtgagtgggccaaagaaaaatgttgttttctgcctctgccatgatcggctcagccaagatgaacttcaatatgggttgcccataaatggaagatttggtctctttttggttgtttcatcttttgcatgaaggtagggaccacataatggcccatcaaaaaattctgctgcagacaactgcagaagacaaatggaacagcttagaaaagggctgatatgattcagaactttcacgagtaggaaaaatggacactacacccatttcagttatcaagttcaagaaagtgggcagggggcaaatcaaaccatgatgcaaagatacttccctttcccccgtctttgtaatattctctctctctctctcttttgttttcctttatttttgttcccaaagacttacctccctcaaccagccatttgtcctgactctgtgtataagctgatgctcagctgctggagaagagatacgaagaaccgtccctcattccaagaaatccaccttctgctccttcaacaaggcgacgagtgatgctgtcagtgcctggccatgttcctacggctcaggtcctccctacaagacctaccactcacccatgcctatgccactccatctggacatttaatgaaactgagagacagaggcttgtttgctttgccctcttttcctggtcacccccactccctacccctgactcatatatacttttttttttttttacattaaagaactaaaaaaggaaaaaaaaaagcctagggcagatacaatctagtaaaagaaaatctttgatataccaaagtgttggataacaaaggctagaaaattcagataatttataaaggttaactatacttgtgcttataaatgtgcagattctacaatatttttccatgtcattctaaaagaatcttcagaaagaaaaacttgaagaatactaatgtcttgggaaacatgagattaagtttagggaaaacacttgatagatgtggagaatctgaggactcagaattcagcaactaatcacaggtggttttctaatttggcctctggacatgtctcactgtttgtatttctctctcctgtcaaagtgaatgatatattcttgaaaacccccaatttcttgaaatgggtgttctgttcattcatgggcaggggtcaggattgaagttcatatatgaaacaactggggatgtagataggaaagaatgtctgctgcaagagtgtatgaagggggattgtcatttacaaaaaaaaagtacctcatggatggaaggattcatgaatagataatggacataggaaaggaggtcaatggaggacacataacagacatgctatccaccatttatttgtgattttgtaagagggttctcttctttgtcttgttcagatattttcatgtgtgtgttttagtgagaatctgagtttttaatcagtaaagtctgatgttttgatatcttgatgttttgatggtagactcttgagttcatcttgtccaaatcttctagcattttcaaggatgaagcctctgtgggatctttgggcttggggctgaatttctcaggcataaatacctaatggccttgtctaagagggaatagatctgttctgagtgtccccaaaaaagttgtcctaggaactgatgtggagaagttacagggagactgatttccattccttattggggaaaacgaagcactcagaacatacaaggataaatgggctgccaccagaggtgaggagtctcttgtcactggagaggttaagatagaggctcaagaacaatgcaaggaaaatggaggttttcaaacacaaagtggatggtggtatgagacaatgtttaatgtccttctaactccgagagtccttgattctggagaggcacaggatgaacatctgttgctactgctgagtatacacactcttgcctctgagatggcgtgaccagcagaaaagaaagctcagggctgtggcttcagaattcatggaaacagagccactgtcaagaaggaatctgctcagagcagattctgagttttcacactccacaaatcctcagatttagtgggaagctggaaaaaggaactcttaattccaattgaggagaaacaagaaaacattacaaatcagtttcccaagctgagaaggattagccttgaccccttgggtctgcctctgttgccctcaccagtttaccttctacaacagtttctcaggaacatgtgtatttccccattcagctttcagcattgctccacgccacagcataagcatagatcccaagtccacaggctccattttgcaggtcatcttctgatcctagcaaatgtcctttccccatagttgtcctatgcctttgggctttagtctatcccaggactaactgtggagaaatcattggtttgagagtcaagagagcattggtttgggagctttaatcctctttctgcttcacactaagtgtgtcatcttggctaaatcacttggtctttctgcattttgttttcttatttataggatgaggaaattagattaaatggttttgaggtcctttcttgttctgatatgtccagtactcactggaaaattggatctataactgatgggtttagtaatctggtcatttcttgctctgaaaattgtagtcagcaaaagagatcatggaagaaatcactgtaatggtagtaatagtaacacatgccatttgtattgtgccttaggtttaccaggtgtttccaaatacattagca

SEQ ID NO: 2 - DNA, 2568 bp, Homo sapiens
  CDS (1)..(2565)
atgatcctgattcccagaatgctcttggtgctgttcctgctgctgcctatcttgagttctgcaaaagctcaggttaatccagctatatgccgctatcctctgggcatgtcaggaggccagattccagatgaggacatcacagcttccagtcagtggtcagagtccacagctgccaaatatggaaggctggactcagaagaaggggatggagcctggtgccctgagattccagtggaacctgatgacctgaaggagtttctgcagattgacttgcacaccctccattttatcactctggtggggacccaggggcgccatgcaggaggtcatggcatcgagtttgcccccatgtacaagatcaattacagtcgggatggcactcgctggatctcttggcggaaccgtcatgggaaacaggtgctggatggaaatagtaacccctatgacattttcctaaaggacttggagccgcccattgtagccagatttgtccggttcattccagtcaccgaccactccatgaatgtgtgtatgagagtggagctttacggctgtgtctggctagatggcttggtgtcttacaatgctccagctgggcagcagtttgtactccctggaggttccatcatttatctgaatgattctgtctatgatggagctgttggatacagcatgacagaagggctaggccaattgaccgatggtgtgtctggcctggacgatttcacccagacccatgaataccacgtgtggcccggctatgactatgtgggctggcggaacgagagtgccaccaatggctacattgagatcatgtttgaatttgaccgcatcaggaatttcactaccatgaaggtccactgcaacaacatgtttgctaaaggtgtgaagatctttaaggaggtacagtgctacttccgctctgaagccagtgagtgggaacctaatgccatttccttcccccttgtcctggatgacgtcaaccccagtgctcggtttgtcacggtgcctctccaccaccgaatggccagtgccatcaagtgtcaataccattttgcagatacctggatgatgttcagtgagatcaccttccaatcagatgctgcaatgtacaacaactctgaagccctgcccacctctcctatggcacccacaacctatgatccaatgcttaaagttgatgacagcaacactcggatcctgattggctgcttggtggccatcatctttatcctcctggccatcattgtcatcatcctctggaggcagttctggcagaaaatgctggagaaggcttctcggaggatgctggatgatgaaatgacagtcagcctttccctgccaagtgattctagcatgttcaacaataaccgctcctcatcacctagtgaacaagggtccaactcgacttacgatcgcatctttccccttcgccctgactaccaggagccatccaggctgatacgaaaactcccagaatttgctccaggggaggaggagtcaggctgcagcggtgttgtgaagccagtccagcccagtggccctgagggggtgccccactatgcagaggctgacatagtgaacctccaaggagtgacaggaggcaacacatactcagtgcctgccgtcaccatggacctgctctcaggaaaagatgtggctgtggaggagttccccaggaaactcctaactttcaaagagaagctgggagaaggacagtttggggaggttcatctctgtgaagtggagggaatggaaaaattcaaagacaaagattttgccctagatgtcagtgccaaccagcctgtcctggtggctgtgaaaatgctccgagcagatgccaacaagaatgccaggaatgattttcttaaggagataaagatcatgtctcggctcaaggacccaaacatcatccatctattagctgtgtgtatcactgatgaccctctctgtatgatcactgaatacatggagaatggagatctcaatcagtttctttcccgccacgagccccctaattcttcctccagcgatgtacgcactgtcagttacaccaatctgaagtttatggctacccaaattgcctctggcatgaagtacctttcctctcttaattttgttcaccgagatctggccacacgaaactgtttagtgggtaagaactacacaatcaagatagctgactttggaatgagcaggaacctgtacagtggtgactattaccggatccagggccgggcagtgctccctatccgctggatgtcttgggagagtatcttgctgggcaagttcactacagcaagtgatgtgtgggcctttggggttactttgtgggagactttcaccttttgtcaagaacagccctattcccagctgtcagatgaacaggttattgagaatactggagagttcttccgagaccaagggaggcagacttacctccctcaaccagccatttgtcctgactctgtgtataagctgatgctcagctgctggagaagagatacgaagaaccgtccctcattccaagaaatccaccttctgctccttcaacaaggcgacgagtga

SEQ ID NO: 3 - protein, 855 aa, Homo sapiens
MILIPRMLLVLFLLLPILSSAKAQVNPAICRYPLGMSGGQIPDEDITASSQWSESTAAKYGRLDSEEGDGAWCPEIPVEPDDLKEFLQIDLHTLHFITLVGTQGRHAGGHGIEFAPMYKINYSRDGTRWISWRNRHGKQVLDGNSNPYDIFLKDLEPPIVARFVRFIPVTDHSMNVCMRVELYGCVWLDGLVSYNAPAGQQFVLPGGSIIYLNDSVYDGAVGYSMTEGLGQLTDGVSGLDDFTQTHEYHVWPGYDYVGWRNESATNGYIEIMFEFDRIRNFTTMKVHCNNMFAKGVKIFKEVQCYFRSEASEWEPNAISFPLVLDDVNPSARFVTVPLHHRMASAIKCQYHFADTWMMFSEITFQSDAAMYNNSEALPTSPMAPTTYDPMLKVDDSNTRILIGCLVAIIFILLAIIVIILWRQFWQKMLEKASRRMLDDEMTVSLSLPSDSSMFNNNRSSSPSEQGSNSTYDRIFPLRPDYQEPSRLIRKLPEFAPGEEESGCSGVVKPVQPSGPEGVPHYAEADIVNLQGVTGGNTYSVPAVTMDLLSGKDVAVEEFPRKLLTFKEKLGEGQFGEVHLCEVEGMEKFKDKDFALDVSANQPVLVAVKMLRADANKNARNDFLKEIKIMSRLKDPNIIHLLAVCITDDPLCMITEYMENGDLNQFLSRHEPPNSSSSDVRTVSYTNLKFMATQIASGMKYLSSLNFVHRDLATRNCLVGKNYTIKIADFGMSRNLYSGDYYRIQGRAVLPIRWMSWESILLGKFTTASDVWAFGVTLWETFTFCQEQPYSQLSDEQVIENTGEFFRDQGRQTYLPQPAICPDSVYKLMLSCWRRDTKNRPSFQEIHLLLLQQGDE

SEQ ID NO: 4 - DNA, 2568 bp, Homo sapiens
  CDS (1)..(2565)
  misc_feature (2041)..(2043): n is a, c, g, or t
atgatcctgattcccagaatgctcttggtgctgttcctgctgctgcctatcttgagttctgcaaaagctcaggttaatccagctatatgccgctatcctctgggcatgtcaggaggccagattccagatgaggacatcacagcttccagtcagtggtcagagtccacagctgccaaatatggaaggctggactcagaagaaggggatggagcctggtgccctgagattccagtggaacctgatgacctgaaggagtttctgcagattgacttgcacaccctccattttatcactctggtggggacccaggggcgccatgcaggaggtcatggcatcgagtttgcccccatgtacaagatcaattacagtcgggatggcactcgctggatctcttggcggaaccgtcatgggaaacaggtgctggatggaaatagtaacccctatgacattttcctaaaggacttggagccgcccattgtagccagatttgtccggttcattccagtcaccgaccactccatgaatgtgtgtatgagagtggagctttacggctgtgtctggctagatggcttggtgtcttacaatgctccagctgggcagcagtttgtactccctggaggttccatcatttatctgaatgattctgtctatgatggagctgttggatacagcatgacagaagggctaggccaattgaccgatggtgtgtctggcctggacgatttcacccagacccatgaataccacgtgtggcccggctatgactatgtgggctggcggaacgagagtgccaccaatggctacattgagatcatgtttgaatttgaccgcatcaggaatttcactaccatgaaggtccactgcaacaacatgtttgctaaaggtgtgaagatctttaaggaggtacagtgctacttccgctctgaagccagtgagtgggaacctaatgccatttccttcccccttgtcctggatgacgtcaaccccagtgctcggtttgtcacggtgcctctccaccaccgaatggccagtgccatcaagtgtcaataccattttgcagatacctggatgatgttcagtgagatcaccttccaatcagatgctgcaatgtacaacaactctgaagccctgcccacctctcctatggcacccacaacctatgatccaatgcttaaagttgatgacagcaacactcggatcctgattggctgcttggtggccatcatctttatcctcctggccatcattgtcatcatcctctggaggcagttctggcagaaaatgctggagaaggcttctcggaggatgctggatgatgaaatgacagtcagcctttccctgccaagtgattctagcatgttcaacaataaccgctcctcatcacctagtgaacaagggtccaactcgacttacgatcgcatctttccccttcgccctgactaccaggagccatccaggctgatacgaaaactcccagaatttgctccaggggaggaggagtcaggctgcagcggtgttgtgaagccagtccagcccagtggccctgagggggtgccccactatgcagaggctgacatagtgaacctccaaggagtgacaggaggcaacacatactcagtgcctgccgtcaccatggacctgctctcaggaaaagatgtggctgtggaggagttccccaggaaactcctaactttcaaagagaagctgggagaaggacagtttggggaggttcatctctgtgaagtggagggaatggaaaaattcaaagacaaagattttgccctagatgtcagtgccaaccagcctgtcctggtggctgtgaaaatgctccgagcagatgccaacaagaatgccaggaatgattttcttaaggagataaagatcatgtctcggctcaaggacccaaacatcatccatctattagctgtgtgtatcactgatgaccctctctgtatgatcactgaatacatggagaatggagatctcaatcagtttctttcccgccacgagccccctaattcttcctccagcgatgtacgcnnngtcagttacaccaatctgaagtttatggctacccaaattgcctctggcatgaagtacctttcctctcttaattttgttcaccgagatctggccacacgaaactgtttagtgggtaagaactacacaatcaagatagctgactttggaatgagcaggaacctgtacagtggtgactattaccggatccagggccgggcagtgctccctatccgctggatgtcttgggagagtatcttgctgggcaagttcactacagcaagtgatgtgtgggcctttggggttactttgtgggagactttcaccttttgtcaagaacagccctattcccagctgtcagatgaacaggttattgagaatactggagagttcttccgagaccaagggaggcagacttacctccctcaaccagccatttgtcctgactctgtgtataagctgatgctcagctgctggagaagagatacgaagaaccgtccctcattccaagaaatccaccttctgctccttcaacaaggcgacgagtga

SEQ ID NO: 5 - protein, 855 aa, Homo sapiens
  misc_feature (681)..(681): The ′Xaa′ at location 681 stands for Lys, Asn, Arg, Ser, Thr, Ile, Met, Glu, Asp, Gly, Ala, Val, Gln, His, Pro, Leu, Tyr, Trp, Cys, or Phe.
MILIPRMLLVLFLLLPILSSAKAQVNPAICRYPLGMSGGQIPDEDITASSQWSESTAAKYGRLDSEEGDGAWCPEIPVEPDDLKEFLQIDLHTLHFITLVGTQGRHAGGHGIEFAPMYKINYSRDGTRWISWRNRHGKQVLDGNSNPYDIFLKDLEPPIVARFVRFIPVTDHSMNVCMRVELYGCVWLDGLVSYNAPAGQQFVLPGGSIIYLNDSVYDGAVGYSMTEGLGQLTDGVSGLDDFTQTHEYHVWPGYDYVGWRNESATNGYIEIMFEFDRIRNFTTMKVHCNNMFAKGVKIFKEVQCYFRSEASEWEPNAISFPLVLDDVNPSARFVTVPLHHRMASAIKCQYHFADTWMMFSEITFQSDAAMYNNSEALPTSPMAPTTYDPMLKVDDSNTRILIGCLVAIIFILLAIIVIILWRQFWQKMLEKASRRMLDDEMTVSLSLPSDSSMFNNNRSSSPSEQGSNSTYDRIFPLRPDYQEPSRLIRKLPEFAPGEEESGCSGVVKPVQPSGPEGVPHYAEADIVNLQGVTGGNTYSVPAVTMDLLSGKDVAVEEFPRKLLTFKEKLGEGQFGEVHLCEVEGMEKFKDKDFALDVSANQPVLVAVKMLRADANKNARNDFLKEIKIMSRLKDPNIIHLLAVCITDDPLCMITEYMENGDLNQFLSRHEPPNSSSSDVRXVSYTNLKFMATQIASGMKYLSSLNFVHRDLATRNCLVGKNYTIKIADFGMSRNLYSGDYYRIQGRAVLPIRWMSWESILLGKFTTASDVWAFGVTLWETFTFCQEQPYSQLSDEQVIENTGEFFRDQGRQTYLPQPAICPDSVYKLMLSCWRRDTKNRPSFQEIHLLLLQQGDE

SEQ ID NO: 6 - DNA, 15 bp, Homo sapiens
tgtacgcaytgtcag